SEQUENCE LISTING (153 sequences)

SEQ ID NO: 1 - protein, 1280 aa, Homo sapiens
SSKYAPDENCPDQNPRLRNWDPGQDSAKQVVIKEGDMLRLTSDATVHSIVIQDGGLLVFGDNKDGSRNITLRTHYILIQDGGALHIGAEKCRYKSKATITLYGKSDEGESMPTFGKKFIGVEAGGTLELHGARKASWTLLARTLNSSGLPFGSYTFEKDFSRGLNVRVIDQDTAKILESERFDTHEYRNESRRLQEFLRFQDPGRIVAIAVGDSAAKSLLQGTIQMIQERLGSELIQGLGYRQAWALVGVIDGGSTSCNESVRNYENHSSGGKALAQREFYTVDGQKFSVTAYSEWIEGVSLSGFRVEVVDGVKLNLLDDVSSWKPGDQIVVASTDYSMYQAEEFTLLPCSECSHFQVKVKETPQFLHMGEIIDGVDMRAEVGILTRNIVIQGEVEDSCYAENQCQFFDYDTFGGHIMIMKNFTSVHLSYVELKHMGQQQMGRYPVHFHLCGDVDYKGGYRHATFVDGLSIHHSFSRCITVHGTNGLLIKDTIGFDTLGHCFFLEDGIEQRNTLFHNLGLLTKPGTLLPTDRNNSMCTTMRDKVFGNYIPVPATDCMAVSTFWIAHPNNNLINNAAAGSQDAGIWYLFHKEPTGESSGLQLLAKPELTPLGIFYNNRVHSNFKAGLFIDKGVKTTNSSAADPREYLCLDNSARFRPHQDANPEKPRVAALIDRLIAFKNNDNGAWVRGGDIIVQNSAFADNGIGLTFASDGSFPSDEGSSQEVSESLFVGESRNYGFQGGQNKYVGTGGIDQKPRTLPRNRTFPIRGFQIYDGPIHLTRSTFKKYVPTPDRYSSAIGFLMKNSWQITPRNNISLVKFGPHVSLNVFFGKPGPWFEDCEMDGDKNSIFHDIDGSVTGYKDAYVGRMDNYLIRHPSCVNVSKWNAVICSGTYAQVYVQTWSTQNLSMTITRDEYPSNPMVLRGINQKAAFPQYQPVVMLEKGYTIHWNGPAPRTTFLYLVNFNKNDWIRVGLCYPSNTSFQVTFGYLQRQNGSLSKIEEYEPVHSLEELQRKQSERKFYFDSSTGLLFLYLKAKSHRHGHSYCSSQGCERVKIQAATDSKDISNCMAKAYPQYYRKPSVVKRMPAMLTGLCQGCGTRQVVFTSDPHKSYLPVQFQSPDKAETQRGDPSVISVNGTDFTFRSAGVLLLVVDPCSVPFRLTEKTVFPLADVSRIEEYLKTGIPPRSIVLLSTRGEIKQLNISHLLVPLGLAKPAHLYDKGSTIFLGFSGNFKPSWTKLFTSPAGQGLGVLEQFIPLQLDEYGCPRATTVRRRDLELLKQASKAH

SEQ ID NO: 2 - DNA, 3840 bp, Homo sapiens
  source 1..3840: mol_type = other DNA
tcctcaaaatatgctccagatgaaaattgcccagatcaaaatcctcgtctcaggaattgggatccaggacaagattctgcaaagcaagttgttatcaaggagggagatatgctccgtctgacctcagacgccaccgtgcattctatagtcattcaggatggaggactgcttgtatttggggacaataaagatggatccagaaatattactttgaggactcattacatcctgatccaggatggtggggcgcttcatattggagcagaaaaatgccgctataaatccaaagcgacaattaccttgtatggcaagtcagatgaaggtgaaagtatgccaacatttggcaaaaagtttattggtgtggaagctggcgggacactggagttacatggggcacggaaggcatcgtggacgttgttggcaaggaccctgaattcctcaggcttgccctttgggtcctatacctttgaaaaggacttttcccggggcctcaatgtgagggtcattgaccaagacacggccaaaattttggaaagtgagagatttgatacccatgaataccgcaatgagagcaggcggcttcaggagtttctgagattccaggatccaggtcggattgttgccatagctgtcggggattcagccgctaaaagtctcttacaaggaaccatccagatgatccaggaacggttgggaagtgaactgatccaaggactgggctacaggcaagcttgggctttagttggtgtcattgatggtggaagcacttcttgcaatgaatccgtgagaaactatgaaaatcatagcagtggcgggaaggctcttgcccaaagagaattttatactgtggatggccagaagttctctgtgacagcttatagtgaatggattgaaggcgtttctctttcaggattccgggtagaggttgtagatggagtgaagctaaatttgctagatgatgttagtagttggaaacctggagaccagattgtggtcgcaagcacagactattccatgtaccaagcagaggagttcactcttcttccctgttctgaatgcagccattttcaggtcaaagtcaaagaaacccctcagttcctgcacatgggtgagatcatagacggtgtagacatgagagctgaggttggaattcttacccggaatattgtgatccaaggagaagtggaggactcatgctacgcagaaaatcagtgccaattttttgattatgatacctttgggggacacattatgataatgaaaaattttacttcagtccatctttcttatgtggaattgaaacacatgggtcagcagcagatggggcgataccctgttcattttcacctgtgtggtgacgtggattataaaggaggatacagacatgcaacatttgtggacggcctgtctattcatcacagcttctcaaggtgcatcactgtgcatgggacaaatggcttgctaataaaagacaccattgggtttgacacactaggtcattgtttctttttggaagatggtattgaacagaggaatactttgttccacaatctgggactcctcaccaagccgggtactctcctgcccaccgataggaacaactccatgtgtaccaccatgcgagataaagtgtttggaaattacattcctgtgcctgctactgactgtatggctgtttcaactttctggattgctcatcccaacaataatctgattaataatgcagctgcaggctcacaggatgctggaatatggtatttattccacaaggaaccaactggggaatccagtggattgcagctcttggcaaaaccagaactcactccattgggtatattttataacaacagggtccattcaaattttaaggctggcttatttattgacaaaggtgtcaaaacaaccaactctagtgctgctgacccaagggaatacctctgtttggacaatagtgcaagatttcgacctcatcaggatgcaaaccccgaaaaaccacgtgttgctgctctaattgacaggctcattgcttttaaaaataatgataatggagcttgggtcagaggaggagatattatcgttcaaaattcagcatttgcagataatggaataggactgacctttgccagtgatggaagcttcccaagtgatgaaggttccagccaagaggtatctgaatctctctttgttggggagagcaggaattacggctttcagggtggtcagaacaagtatgtaggcactggaggaatagaccagaagcctcgaacattacccaggaacaggacgttcccaattagaggctttcagatttatgatgggcccattcatctcacaaggagcactttcaaaaaatatgtgccaactccagataggtacagcagtgcaattggcttcctcatgaagaattcctggcagataacccccaggaataatatctccctcgtgaagtttggtccacatgtctctctgaatgtcttttttggaaagcctggtccctggtttgaagattgtgagatggatggtgataagaactccatattccatgacattgatggctctgtgacaggatacaaggatgcttatgtgggaagaatggacaactacctgatccgccatccaagctgtgtaaatgtgtctaagtggaatgcagtgatctgcagtgggacctatgcacaggtctatgtacagacatggagcactcagaatctttctatgaccattacacgagatgagtatccgtccaaccctatggtgctccgaggtattaatcagaaggctgcctttccacagtaccagcctgtcgtcatgctggagaagggttataccatccactggaatgggccggcaccacggactacatttctatacctcgtcaacttcaacaagaatgactggattcgagttggcctttgctatccatcaaacacaagttttcaagttacctttggctatttgcagcggcagaatggctcattatccaaaatcgaagaatatgagcctgtgcattcactggaagaactgcaaagaaagcaatccgagaggaaattctattttgactccagcacggggttactgtttttgtatctcaaagccaaaagccacaggcatggccacagttactgttcatctcagggatgtgaaagagtcaagatccaagcagccacagactcaaaggacatcagtaactgcatggccaaagcatacccacagtactacagaaagccgtcagtggtcaagcggatgccggccatgctcactggactctgtcaaggctgtggcactcggcaggtggtgtttactagtgatcctcataaaagttacctccctgtgcaattccagtcacctgataaagcagaaacccagcgtggagacccgtctgttatttctgtcaatggcactgactttaccttccgaagtgcaggcgtcctcctccttgttgtggatccgtgcagcgttccattccgcttgacggaaaaaacggtttttcctcttgctgatgtcagtcgcattgaagagtatttaaaaacaggcatccctccaaggtccattgttctgttgagcacaagaggagaaataaagcagttaaacatttcacacttactagtacctctgggattagccaaaccagctcatctttatgacaaagggagtaccatatttttgggattcagtggaaactttaaaccatcatggactaagctatttaccagtcctgctggacagggccttggggtgcttgaacaattcatacctttgcagctggacgaatatggttgtcccagagccaccactgtccgcagaagagacctggaactgctaaagcaagcttcaaaagcacat

SEQ ID NO: 3 - protein, 1383 aa, Homo sapiens
MYATDSRGHSPAFLQPQNGNSRHPSGYVPGKVVPLRPPPPPKSQASAKFTSIRREDRATFAFSPEEQQAQRESQKQKRHKNTFICFAITSFSFFIALAIILGISSKYAPDENCPDQNPRLRNWDPGQDSAKQVVIKEGDMLRLTSDATVHSIVIQDGGLLVFGDNKDGSRNITLRTHYILIQDGGALHIGAEKCRYKSKATITLYGKSDEGESMPTFGKKFIGVEAGGTLELHGARKASWTLLARTLNSSGLPFGSYTFEKDFSRGLNVRVIDQDTAKILESERFDTHEYRNESRRLQEFLRFQDPGRIVAIAVGDSAAKSLLQGTIQMIQERLGSELIQGLGYRQAWALVGVIDGGSTSCNESVRNYENHSSGGKALAQREFYTVDGQKFSVTAYSEWIEGVSLSGFRVEVVDGVKLNLLDDVSSWKPGDQIVVASTDYSMYQAEEFTLLPCSECSHFQVKVKETPQFLHMGEIIDGVDMRAEVGILTRNIVIQGEVEDSCYAENQCQFFDYDTFGGHIMIMKNFTSVHLSYVELKHMGQQQMGRYPVHFHLCGDVDYKGGYRHATFVDGLSIHHSFSRCITVHGTNGLLIKDTIGFDTLGHCFFLEDGIEQRNTLFHNLGLLTKPGTLLPTDRNNSMCTTMRDKVFGNYIPVPATDCMAVSTFWIAHPNNNLINNAAAGSQDAGIWYLFHKEPTGESSGLQLLAKPELTPLGIFYNNRVHSNFKAGLFIDKGVKTTNSSAADPREYLCLDNSARFRPHQDANPEKPRVAALIDRLIAFKNNDNGAWVRGGDIIVQNSAFADNGIGLTFASDGSFPSDEGSSQEVSESLFVGESRNYGFQGGQNKYVGTGGIDQKPRTLPRNRTFPIRGFQIYDGPIHLTRSTFKKYVPTPDRYSSAIGFLMKNSWQITPRNNISLVKFGPHVSLNVFFGKPGPWFEDCEMDGDKNSIFHDIDGSVTGYKDAYVGRMDNYLIRHPSCVNVSKWNAVICSGTYAQVYVQTWSTQNLSMTITRDEYPSNPMVLRGINQKAAFPQYQPVVMLEKGYTIHWNGPAPRTTFLYLVNFNKNDWIRVGLCYPSNTSFQVTFGYLQRQNGSLSKIEEYEPVHSLEELQRKQSERKFYFDSSTGLLFLYLKAKSHRHGHSYCSSQGCERVKIQAATDSKDISNCMAKAYPQYYRKPSVVKRMPAMLTGLCQGCGTRQVVFTSDPHKSYLPVQFQSPDKAETQRGDPSVISVNGTDFTFRSAGVLLLVVDPCSVPFRLTEKTVFPLADVSRIEEYLKTGIPPRSIVLLSTRGEIKQLNISHLLVPLGLAKPAHLYDKGSTIFLGFSGNFKPSWTKLFTSPAGQGLGVLEQFIPLQLDEYGCPRATTVRRRDLELLKQASKAH

SEQ ID NO: 4 - DNA, 6152 bp, Homo sapiens
  source 1..6152: mol_type = other DNA
agttgagtacgagcggcagctgcgggcgccggcaatcgagtggcttcctggccatggagaggagatctggacccgaggacgccagggactcctctccgtctccctggaccgctgtgtaggcacctgagttaacggacgctaagcggggtaggggaagtaacagtgtatcatgtatgccactgattccaggggacactcccctgctttcctccaacctcagaatggaaatagtcgtcacccatctggctatgttccagggaaggttgtcccattgcgtccccctcctcctccaaagagtcaagcttcagccaaatttacctccatcagacgagaagaccgggcaaccttcgcattctcacctgaagaacagcaagcccagagagaaagtcaaaagcaaaagagacacaaaaatactttcatttgttttgctattactagtttctcattttttattgcacttgcaatcattttaggaatatcctcaaaatatgctccagatgaaaattgcccagatcaaaatcctcgtctcaggaattgggatccaggacaagattctgcaaagcaagttgttatcaaggagggagatatgctccgtctgacctcagacgccaccgtgcattctatagtcattcaggatggaggactgcttgtatttggggacaataaagatggatccagaaatattactttgaggactcattacatcctgatccaggatggtggggcgcttcatattggagcagaaaaatgccgctataaatccaaagcgacaattaccttgtatggcaagtcagatgaaggtgaaagtatgccaacatttggcaaaaagtttattggtgtggaagctggcgggacactggagttacatggggcacggaaggcatcgtggacgttgttggcaaggaccctgaattcctcaggcttgccctttgggtcctatacctttgaaaaggacttttcccggggcctcaatgtgagggtcattgaccaagacacggccaaaattttggaaagtgagagatttgatacccatgaataccgcaatgagagcaggcggcttcaggagtttctgagattccaggatccaggtcggattgttgccatagctgtcggggattcagccgctaaaagtctcttacaaggaaccatccagatgatccaggaacggttgggaagtgaactgatccaaggactgggctacaggcaagcttgggctttagttggtgtcattgatggtggaagcacttcttgcaatgaatccgtgagaaactatgaaaatcatagcagtggcgggaaggctcttgcccaaagagaattttatactgtggatggccagaagttctctgtgacagcttatagtgaatggattgaaggcgtttctctttcaggattccgggtagaggttgtagatggagtgaagctaaatttgctagatgatgttagtagttggaaacctggagaccagattgtggtcgcaagcacagactattccatgtaccaagcagaggagttcactcttcttccctgttctgaatgcagccattttcaggtcaaagtcaaagaaacccctcagttcctgcacatgggtgagatcatagacggtgtagacatgagagctgaggttggaattcttacccggaatattgtgatccaaggagaagtggaggactcatgctacgcagaaaatcagtgccaattttttgattatgatacctttgggggacacattatgataatgaaaaattttacttcagtccatctttcttatgtggaattgaaacacatgggtcagcagcagatggggcgataccctgttcattttcacctgtgtggtgacgtggattataaaggaggatacagacatgcaacatttgtggacggcctgtctattcatcacagcttctcaaggtgcatcactgtgcatgggacaaatggcttgctaataaaagacaccattgggtttgacacactaggtcattgtttctttttggaagatggtattgaacagaggaatactttgttccacaatctgggactcctcaccaagccgggtactctcctgcccaccgataggaacaactccatgtgtaccaccatgcgagataaagtgtttggaaattacattcctgtgcctgctactgactgtatggctgtttcaactttctggattgctcatcccaacaataatctgattaataatgcagctgcaggctcacaggatgctggaatatggtatttattccacaaggaaccaactggggaatccagtggattgcagctcttggcaaaaccagaactcactccattgggtatattttataacaacagggtccattcaaattttaaggctggcttatttattgacaaaggtgtcaaaacaaccaactctagtgctgctgacccaagggaatacctctgtttggacaatagtgcaagatttcgacctcatcaggatgcaaaccccgaaaaaccacgtgttgctgctctaattgacaggctcattgcttttaaaaataatgataatggagcttgggtcagaggaggagatattatcgttcaaaattcagcatttgcagataatggaataggactgacctttgccagtgatggaagcttcccaagtgatgaaggttccagccaagaggtatctgaatctctctttgttggggagagcaggaattacggctttcagggtggtcagaacaagtatgtaggcactggaggaatagaccagaagcctcgaacattacccaggaacaggacgttcccaattagaggctttcagatttatgatgggcccattcatctcacaaggagcactttcaaaaaatatgtgccaactccagataggtacagcagtgcaattggcttcctcatgaagaattcctggcagataacccccaggaataatatctccctcgtgaagtttggtccacatgtctctctgaatgtcttttttggaaagcctggtccctggtttgaagattgtgagatggatggtgataagaactccatattccatgacattgatggctctgtgacaggatacaaggatgcttatgtgggaagaatggacaactacctgatccgccatccaagctgtgtaaatgtgtctaagtggaatgcagtgatctgcagtgggacctatgcacaggtctatgtacagacatggagcactcagaatctttctatgaccattacacgagatgagtatccgtccaaccctatggtgctccgaggtattaatcagaaggctgcctttccacagtaccagcctgtcgtcatgctggagaagggttataccatccactggaatgggccggcaccacggactacatttctatacctcgtcaacttcaacaagaatgactggattcgagttggcctttgctatccatcaaacacaagttttcaagttacctttggctatttgcagcggcagaatggctcattatccaaaatcgaagaatatgagcctgtgcattcactggaagaactgcaaagaaagcaatccgagaggaaattctattttgactccagcacggggttactgtttttgtatctcaaagccaaaagccacaggcatggccacagttactgttcatctcagggatgtgaaagagtcaagatccaagcagccacagactcaaaggacatcagtaactgcatggccaaagcatacccacagtactacagaaagccgtcagtggtcaagcggatgccggccatgctcactggactctgtcaaggctgtggcactcggcaggtggtgtttactagtgatcctcataaaagttacctccctgtgcaattccagtcacctgataaagcagaaacccagcgtggagacccgtctgttatttctgtcaatggcactgactttaccttccgaagtgcaggcgtcctcctccttgttgtggatccgtgcagcgttccattccgcttgacggaaaaaacggtttttcctcttgctgatgtcagtcgcattgaagagtatttaaaaacaggcatccctccaaggtccattgttctgttgagcacaagaggagaaataaagcagttaaacatttcacacttactagtacctctgggattagccaaaccagctcatctttatgacaaagggagtaccatatttttgggattcagtggaaactttaaaccatcatggactaagctatttaccagtcctgctggacagggccttggggtgcttgaacaattcatacctttgcagctggacgaatatggttgtcccagagccaccactgtccgcagaagagacctggaactgctaaagcaagcttcaaaagcacattagagactaactgtaacttaagtgctgggggaaaaaaaatgtgaactaacttatttaatttatggcattttaaaatgacactgttaacccaacggaaccattttccagtttgatacagaatggggagaaaagaaagcgtttgaaattattgcttggataccagcttcatgcaccttctagttgtacaaaatgttaaagacgttgtttgtatttgtaaggctggtgtattcagagagcagatctcttattcctcactttccacccccgtattttgtaatgaccatgagcaatgtttttactttttgtataatggggtggggtggagtgggggcttctgagagtcagcctgaggtctttagaggaccagctattgtagcaccttggatacttgaagtttaatgctcagttgggtcgggtggcagttgacttggtggctggcatgttcagcagtgcctggggccctgtttctgggcagcctttgaggattttctatgatattgaatgacagttttaagtggcaactcaggcccagctcatgcccttttttgcctggacatgtgctatttttattcacttatatatcaattacttgtaagggttaaactttcaaacaggaagtatattgggacaaaagggctcttggggattagatatccctttaatctgtgaccattgggcaaaaaattttcctgcagcaaaagtctgaggctgttgggaccatttttgcagctttaatccttagcctcttttgactgtatatttgtgtttaaaatgcagagctcaactgaatatttccttttttgttttgttttgttttgttttaagaagtaggttgttttcctgaaccgtaaacttgtatcattttaacttgcacaaaggaagtctgttcttggtattgctcttgcacttgggttttttgttattgttttgtgtggattttttaaagcttttctgttcaccctcctgccaggaaaatcccagaaagcttaatgataccccaaaatgattacacccagggaggaaaaaaaggagcgctttctagggtcagaatcgtggagagaatactcagaaatgaacctctttaaagccttgcaggaatgagtcactcttacttaatgaaatgttaaagccaattaaaaagcatgctgtgatgcccagcttccctttccacagggtgcatgcgtctcctgctggtgaatcacatgcggcaagaggcaactggctccacagcctgggatgctgccgtaccaagaggaaagaagcagcaaaatgcctttacgttgttctaaacccccgacgcataaagtgtagaggagggatggccaagggtgggtggtagaaagtgtgttcaggctgacactggcaatgagtacagataatttcactttcctcttctaggggcaaaggctgatggcctctacctttgtatccaggagaaactgcagagcagccctgtgactttacaaaatatgctacctcaaagtgctaccgataaacctttctaattgtaagtgcccttactaagggcacatgtcttaatcaaagttagttttttgttttctggtttgttttttttttttgtatattgatgaatgagatcttacctattaaatatattattggattatggttcctgaaggtcattagagtgtgtgtgtgtgtgtgtgtgtgtgtgtgtgttttatgacttaaatatctttacgtgtgttttttagagcttggttctttaaagatttggagaagatatgtaaattaccaaggcacttggtttttctgttttatatactaataatcagggcctaagttaaataaaaatatgtgtgcatgtatttta

SEQ ID NO: 5 - protein, 18 aa, Homo sapiens
GSSENLYFQGSSHHHHHH

SEQ ID NO: 6 - protein, 12 aa, Homo sapiens
GSSENLYFQGSS

SEQ ID NO: 7 - protein, 23 aa, Homo sapiens
APLVHHHHHHALDENLYFQGALA

SEQ ID NO: 8 - protein, 16 aa, Homo sapiens
DAAQPARRARRTKLGT

SEQ ID NO: 9 - protein, 6 aa, Synthetic construct
HHHHHH

SEQ ID NO: 10 - protein, 1314 aa, Synthetic construct
DAAQPARRARRTKLGTSSKYAPDENCPDQNPRLRNWDPGQDSAKQVVIKEGDMLRLTSDATVHSIVIQDGGLLVFGDNKDGSRNITLRTHYILIQDGGALHIGAEKCRYKSKATITLYGKSDEGESMPTFGKKFIGVEAGGTLELHGARKASWTLLARTLNSSGLPFGSYTFEKDFSRGLNVRVIDQDTAKILESERFDTHEYRNESRRLQEFLRFQDPGRIVAIAVGDSAAKSLLQGTIQMIQERLGSELIQGLGYRQAWALVGVIDGGSTSCNESVRNYENHSSGGKALAQREFYTVDGQKFSVTAYSEWIEGVSLSGFRVEVVDGVKLNLLDDVSSWKPGDQIVVASTDYSMYQAEEFTLLPCSECSHFQVKVKETPQFLHMGEIIDGVDMRAEVGILTRNIVIQGEVEDSCYAENQCQFFDYDTFGGHIMIMKNFTSVHLSYVELKHMGQQQMGRYPVHFHLCGDVDYKGGYRHATFVDGLSIHHSFSRCITVHGTNGLLIKDTIGFDTLGHCFFLEDGIEQRNTLFHNLGLLTKPGTLLPTDRNNSMCTTMRDKVFGNYIPVPATDCMAVSTFWIAHPNNNLINNAAAGSQDAGIWYLFHKEPTGESSGLQLLAKPELTPLGIFYNNRVHSNFKAGLFIDKGVKTTNSSAADPREYLCLDNSARFRPHQDANPEKPRVAALIDRLIAFKNNDNGAWVRGGDIIVQNSAFADNGIGLTFASDGSFPSDEGSSQEVSESLFVGESRNYGFQGGQNKYVGTGGIDQKPRTLPRNRTFPIRGFQIYDGPIHLTRSTFKKYVPTPDRYSSAIGFLMKNSWQITPRNNISLVKFGPHVSLNVFFGKPGPWFEDCEMDGDKNSIFHDIDGSVTGYKDAYVGRMDNYLIRHPSCVNVSKWNAVICSGTYAQVYVQTWSTQNLSMTITRDEYPSNPMVLRGINQKAAFPQYQPVVMLEKGYTIHWNGPAPRTTFLYLVNFNKNDWIRVGLCYPSNTSFQVTFGYLQRQNGSLSKIEEYEPVHSLEELQRKQSERKFYFDSSTGLLFLYLKAKSHRHGHSYCSSQGCERVKIQAATDSKDISNCMAKAYPQYYRKPSVVKRMPAMLTGLCQGCGTRQVVFTSDPHKSYLPVQFQSPDKAETQRGDPSVISVNGTDFTFRSAGVLLLVVDPCSVPFRLTEKTVFPLADVSRIEEYLKTGIPPRSIVLLSTRGEIKQLNISHLLVPLGLAKPAHLYDKGSTIFLGFSGNFKPSWTKLFTSPAGQGLGVLEQFIPLQLDEYGCPRATTVRRRDLELLKQASKAHGSSENLYFQGSSHHHHHH

SEQ ID NO: 11 - DNA, 4011 bp, Synthetic construct
  source 1..4011: mol_type = other DNA
atggagacagacacactcctgctatgggtactgctgctctgggttccaggttccactggtgacgcggcccagccggccaggcgcgcgcgccgtacgaagcttggtacctcctcaaaatatgctccagatgaaaattgcccagatcaaaatcctcgtctcaggaattgggatccaggacaagattctgcaaagcaagttgttatcaaggagggagatatgctccgtctgacctcagacgccaccgtgcattctatagtcattcaggatggaggactgcttgtatttggggacaataaagatggatccagaaatattactttgaggactcattacatcctgatccaggatggtggggcgcttcatattggagcagaaaaatgccgctataaatccaaagcgacaattaccttgtatggcaagtcagatgaaggtgaaagtatgccaacatttggcaaaaagtttattggtgtggaagctggcgggacactggagttacatggggcacggaaggcatcgtggacgttgttggcaaggaccctgaattcctcaggcttgccctttgggtcctatacctttgaaaaggacttttcccggggcctcaatgtgagggtcattgaccaagacacggccaaaattttggaaagtgagagatttgatacccatgaataccgcaatgagagcaggcggcttcaggagtttctgagattccaggatccaggtcggattgttgccatagctgtcggggattcagccgctaaaagtctcttacaaggaaccatccagatgatccaggaacggttgggaagtgaactgatccaaggactgggctacaggcaagcttgggctttagttggtgtcattgatggtggaagcacttcttgcaatgaatccgtgagaaactatgaaaatcatagcagtggcgggaaggctcttgcccaaagagaattttatactgtggatggccagaagttctctgtgacagcttatagtgaatggattgaaggcgtttctctttcaggattccgggtagaggttgtagatggagtgaagctaaatttgctagatgatgttagtagttggaaacctggagaccagattgtggtcgcaagcacagactattccatgtaccaagcagaggagttcactcttcttccctgttctgaatgcagccattttcaggtcaaagtcaaagaaacccctcagttcctgcacatgggtgagatcatagacggtgtagacatgagagctgaggttggaattcttacccggaatattgtgatccaaggagaagtggaggactcatgctacgcagaaaatcagtgccaattttttgattatgatacctttgggggacacattatgataatgaaaaattttacttcagtccatctttcttatgtggaattgaaacacatgggtcagcagcagatggggcgataccctgttcattttcacctgtgtggtgacgtggattataaaggaggatacagacatgcaacatttgtggacggcctgtctattcatcacagcttctcaaggtgcatcactgtgcatgggacaaatggcttgctaataaaagacaccattgggtttgacacactaggtcattgtttctttttggaagatggtattgaacagaggaatactttgttccacaatctgggactcctcaccaagccgggtactctcctgcccaccgataggaacaactccatgtgtaccaccatgcgagataaagtgtttggaaattacattcctgtgcctgctactgactgtatggctgtttcaactttctggattgctcatcccaacaataatctgattaataatgcagctgcaggctcacaggatgctggaatatggtatttattccacaaggaaccaactggggaatccagtggattgcagctcttggcaaaaccagaactcactccattgggtatattttataacaacagggtccattcaaattttaaggctggcttatttattgacaaaggtgtcaaaacaaccaactctagtgctgctgacccaagggaatacctctgtttggacaatagtgcaagatttcgacctcatcaggatgcaaaccccgaaaaaccacgtgttgctgctctaattgacaggctcattgcttttaaaaataatgataatggagcttgggtcagaggaggagatattatcgttcaaaattcagcatttgcagataatggaataggactgacctttgccagtgatggaagcttcccaagtgatgaaggttccagccaagaggtatctgaatctctctttgttggggagagcaggaattacggctttcagggtggtcagaacaagtatgtaggcactggaggaatagaccagaagcctcgaacattacccaggaacaggacgttcccaattagaggctttcagatttatgatgggcccattcatctcacaaggagcactttcaaaaaatatgtgccaactccagataggtacagcagtgcaattggcttcctcatgaagaattcctggcagataacccccaggaataatatctccctcgtgaagtttggtccacatgtctctctgaatgtcttttttggaaagcctggtccctggtttgaagattgtgagatggatggtgataagaactccatattccatgacattgatggctctgtgacaggatacaaggatgcttatgtgggaagaatggacaactacctgatccgccatccaagctgtgtaaatgtgtctaagtggaatgcagtgatctgcagtgggacctatgcacaggtctatgtacagacatggagcactcagaatctttctatgaccattacacgagatgagtatccgtccaaccctatggtgctccgaggtattaatcagaaggctgcctttccacagtaccagcctgtcgtcatgctggagaagggttataccatccactggaatgggccggcaccacggactacatttctatacctcgtcaacttcaacaagaatgactggattcgagttggcctttgctatccatcaaacacaagttttcaagttacctttggctatttgcagcggcagaatggctcattatccaaaatcgaagaatatgagcctgtgcattcactggaagaactgcaaagaaagcaatccgagaggaaattctattttgactccagcacggggttactgtttttgtatctcaaagccaaaagccacaggcatggccacagttactgttcatctcagggatgtgaaagagtcaagatccaagcagccacagactcaaaggacatcagtaactgcatggccaaagcatacccacagtactacagaaagccgtcagtggtcaagcggatgccggccatgctcactggactctgtcaaggctgtggcactcggcaggtggtgtttactagtgatcctcataaaagttacctccctgtgcaattccagtcacctgataaagcagaaacccagcgtggagacccgtctgttatttctgtcaatggcactgactttaccttccgaagtgcaggcgtcctcctccttgttgtggatccgtgcagcgttccattccgcttgacggaaaaaacggtttttcctcttgctgatgtcagtcgcattgaagagtatttaaaaacaggcatccctccaaggtccattgttctgttgagcacaagaggagaaataaagcagttaaacatttcacacttactagtacctctgggattagccaaaccagctcatctttatgacaaagggagtaccatatttttgggattcagtggaaactttaaaccatcatggactaagctatttaccagtcctgctggacagggccttggggtgcttgaacaattcatacctttgcagctggacgaatatggttgtcccagagccaccactgtccgcagaagagacctggaactgctaaagcaagcttcaaaagcacatggttctagcgaaaacctgtattttcagggctctagccatcatcaccatcaccattgactcgag

SEQ ID NO: 12-19 - skipped (8 entries)

SEQ ID NO: 20 - protein, 10 aa, Homo sapiens
SSKYAPDENC

SEQ ID NO: 21 - protein, 10 aa, Homo sapiens
PDQNPRLRNW

SEQ ID NO: 22 - protein, 10 aa, Homo sapiens
DPGQDSAKQV

SEQ ID NO: 23 - protein, 10 aa, Homo sapiens
VIKEGDMLRL

SEQ ID NO: 24 - protein, 10 aa, Homo sapiens
TSDATVHSIV

SEQ ID NO: 25 - protein, 10 aa, Homo sapiens
IQDGGLLVFG

SEQ ID NO: 26 - protein, 10 aa, Homo sapiens
DNKDGSRNIT

SEQ ID NO: 27 - protein, 10 aa, Homo sapiens
LRTHYILIQD

SEQ ID NO: 28 - protein, 10 aa, Homo sapiens
GGALHIGAEK

SEQ ID NO: 29 - protein, 10 aa, Homo sapiens
CRYKSKATIT

SEQ ID NO: 30 - protein, 10 aa, Homo sapiens
LYGKSDEGES

SEQ ID NO: 31 - protein, 10 aa, Homo sapiens
MPTFGKKFIG

SEQ ID NO: 32 - protein, 10 aa, Homo sapiens
VEAGGTLELH

SEQ ID NO: 33 - protein, 10 aa, Homo sapiens
GARKASWTLL

SEQ ID NO: 34 - protein, 10 aa, Homo sapiens
ARTLNSSGLP

SEQ ID NO: 35 - protein, 10 aa, Homo sapiens
FGSYTFEKDF

SEQ ID NO: 36 - protein, 10 aa, Homo sapiens
SRGLNVRVID

SEQ ID NO: 37 - protein, 10 aa, Homo sapiens
QDTAKILESE

SEQ ID NO: 38 - protein, 10 aa, Homo sapiens
RFDTHEYRNE

SEQ ID NO: 39 - protein, 10 aa, Homo sapiens
SRRLQEFLRF

SEQ ID NO: 40 - protein, 10 aa, Homo sapiens
QDPGRIVAIA

SEQ ID NO: 41 - protein, 10 aa, Homo sapiens
VGDSAAKSLL

SEQ ID NO: 42 - protein, 10 aa, Homo sapiens
QGTIQMIQER

SEQ ID NO: 43 - protein, 10 aa, Homo sapiens
LGSELIQGLG

SEQ ID NO: 44 - protein, 10 aa, Homo sapiens
YRQAWALVGV

SEQ ID NO: 45 - protein, 10 aa, Homo sapiens
IDGGSTSCNE

SEQ ID NO: 46 - protein, 10 aa, Homo sapiens
SVRNYENHSS

SEQ ID NO: 47 - protein, 10 aa, Homo sapiens
GGKALAQREF

SEQ ID NO: 48 - protein, 10 aa, Homo sapiens
YTVDGQKFSV

SEQ ID NO: 49 - protein, 10 aa, Homo sapiens
TAYSEWIEGV

SEQ ID NO: 50 - protein, 10 aa, Homo sapiens
SLSGFRVEVV

SEQ ID NO: 51 - protein, 10 aa, Homo sapiens
DGVKLNLLDD

SEQ ID NO: 52 - protein, 10 aa, Homo sapiens
VSSWKPGDQI

SEQ ID NO: 53 - protein, 10 aa, Homo sapiens
VVASTDYSMY

SEQ ID NO: 54 - protein, 10 aa, Homo sapiens
QAEEFTLLPC

SEQ ID NO: 55 - protein, 10 aa, Homo sapiens
SECSHFQVKV

SEQ ID NO: 56 - protein, 10 aa, Homo sapiens
KETPQFLHMG

SEQ ID NO: 57 - protein, 10 aa, Homo sapiens
EIIDGVDMRA

SEQ ID NO: 58 - protein, 10 aa, Homo sapiens
EVGILTRNIV

SEQ ID NO: 59 - protein, 10 aa, Homo sapiens
IQGEVEDSCY

SEQ ID NO: 60 - protein, 10 aa, Homo sapiens
AENQCQFFDY

SEQ ID NO: 61 - protein, 10 aa, Homo sapiens
DTFGGHIMIM

SEQ ID NO: 62 - protein, 10 aa, Homo sapiens
KNFTSVHLSY

SEQ ID NO: 63 - protein, 10 aa, Homo sapiens
VELKHMGQQQ

SEQ ID NO: 64 - protein, 10 aa, Homo sapiens
MGRYPVHFHL

SEQ ID NO: 65 - protein, 10 aa, Homo sapiens
CGDVDYKGGY

SEQ ID NO: 66 - protein, 10 aa, Homo sapiens
RHATFVDGLS

SEQ ID NO: 67 - protein, 10 aa, Homo sapiens
IHHSFSRCIT

SEQ ID NO: 68 - protein, 10 aa, Homo sapiens
VHGTNGLLIK

SEQ ID NO: 69 - protein, 10 aa, Homo sapiens
DTIGFDTLGH

SEQ ID NO: 70 - protein, 10 aa, Homo sapiens
CFFLEDGIEQ

SEQ ID NO: 71 - protein, 10 aa, Homo sapiens
RNTLFHNLGL

SEQ ID NO: 72 - protein, 10 aa, Homo sapiens
LTKPGTLLPT

SEQ ID NO: 73 - protein, 10 aa, Homo sapiens
DRNNSMCTTM

SEQ ID NO: 74 - protein, 10 aa, Homo sapiens
RDKVFGNYIP

SEQ ID NO: 75 - protein, 10 aa, Homo sapiens
VPATDCMAVS

SEQ ID NO: 76 - protein, 10 aa, Homo sapiens
TFWIAHPNNN

SEQ ID NO: 77 - protein, 10 aa, Homo sapiens
LINNAAAGSQ

SEQ ID NO: 78 - protein, 10 aa, Homo sapiens
DAGIWYLFHK

SEQ ID NO: 79 - protein, 10 aa, Homo sapiens
EPTGESSGLQ

SEQ ID NO: 80 - protein, 10 aa, Homo sapiens
LLAKPELTPL

SEQ ID NO: 81 - protein, 10 aa, Homo sapiens
GIFYNNRVHS

SEQ ID NO: 82 - protein, 10 aa, Homo sapiens
NFKAGLFIDK

SEQ ID NO: 83 - protein, 10 aa, Homo sapiens
GVKTTNSSAA

SEQ ID NO: 84 - protein, 10 aa, Homo sapiens
DPREYLCLDN

SEQ ID NO: 85 - protein, 10 aa, Homo sapiens
SARFRPHQDA

SEQ ID NO: 86 - protein, 10 aa, Homo sapiens
NPEKPRVAAL

SEQ ID NO: 87 - protein, 10 aa, Homo sapiens
IDRLIAFKNN

SEQ ID NO: 88 - protein, 10 aa, Homo sapiens
DNGAWVRGGD

SEQ ID NO: 89 - protein, 10 aa, Homo sapiens
IIVQNSAFAD

SEQ ID NO: 90 - protein, 10 aa, Homo sapiens
NGIGLTFASD

SEQ ID NO: 91 - protein, 10 aa, Homo sapiens
GSFPSDEGSS

SEQ ID NO: 92 - protein, 10 aa, Homo sapiens
QEVSESLFVG

SEQ ID NO: 93 - protein, 10 aa, Homo sapiens
ESRNYGFQGG

SEQ ID NO: 94 - protein, 10 aa, Homo sapiens
QNKYVGTGGI

SEQ ID NO: 95 - protein, 10 aa, Homo sapiens
DQKPRTLPRN

SEQ ID NO: 96 - protein, 10 aa, Homo sapiens
RTFPIRGFQI

SEQ ID NO: 97 - protein, 10 aa, Homo sapiens
YDGPIHLTRS

SEQ ID NO: 98 - protein, 10 aa, Homo sapiens
TFKKYVPTPD

SEQ ID NO: 99 - protein, 10 aa, Homo sapiens
RYSSAIGFLM

SEQ ID NO: 100 - protein, 10 aa, Homo sapiens
KNSWQITPRN

SEQ ID NO: 101 - protein, 10 aa, Homo sapiens
NISLVKFGPH

SEQ ID NO: 102 - protein, 10 aa, Homo sapiens
VSLNVFFGKP

SEQ ID NO: 103 - protein, 10 aa, Homo sapiens
GPWFEDCEMD

SEQ ID NO: 104 - protein, 10 aa, Homo sapiens
GDKNSIFHDI

SEQ ID NO: 105 - protein, 10 aa, Homo sapiens
DGSVTGYKDA

SEQ ID NO: 106 - protein, 10 aa, Homo sapiens
YVGRMDNYLI

SEQ ID NO: 107 - protein, 10 aa, Homo sapiens
RHPSCVNVSK

SEQ ID NO: 108 - protein, 10 aa, Homo sapiens
WNAVICSGTY

SEQ ID NO: 109 - protein, 10 aa, Homo sapiens
AQVYVQTWST

SEQ ID NO: 110 - protein, 10 aa, Homo sapiens
QNLSMTITRD

SEQ ID NO: 111 - protein, 10 aa, Homo sapiens
EYPSNPMVLR

SEQ ID NO: 112 - protein, 10 aa, Homo sapiens
GINQKAAFPQ

SEQ ID NO: 113 - protein, 10 aa, Homo sapiens
YQPVVMLEKG

SEQ ID NO: 114 - protein, 10 aa, Homo sapiens
YTIHWNGPAP

SEQ ID NO: 115 - protein, 10 aa, Homo sapiens
RTTFLYLVNF

SEQ ID NO: 116 - protein, 10 aa, Homo sapiens
NKNDWIRVGL

SEQ ID NO: 117 - protein, 10 aa, Homo sapiens
CYPSNTSFQV

SEQ ID NO: 118 - protein, 10 aa, Homo sapiens
TFGYLQRQNG

SEQ ID NO: 119 - protein, 10 aa, Homo sapiens
SLSKIEEYEP

SEQ ID NO: 120 - protein, 10 aa, Homo sapiens
VHSLEELQRK

SEQ ID NO: 121 - protein, 10 aa, Homo sapiens
QSERKFYFDS

SEQ ID NO: 122 - protein, 10 aa, Homo sapiens
STGLLFLYLK

SEQ ID NO: 123 - protein, 10 aa, Homo sapiens
AKSHRHGHSY

SEQ ID NO: 124 - protein, 10 aa, Homo sapiens
CSSQGCERVK

SEQ ID NO: 125 - protein, 10 aa, Homo sapiens
IQAATDSKDI

SEQ ID NO: 126 - protein, 10 aa, Homo sapiens
SNCMAKAYPQ

SEQ ID NO: 127 - protein, 10 aa, Homo sapiens
YYRKPSVVKR

SEQ ID NO: 128 - protein, 10 aa, Homo sapiens
MPAMLTGLCQ

SEQ ID NO: 129 - protein, 10 aa, Homo sapiens
GCGTRQVVFT

SEQ ID NO: 130 - protein, 10 aa, Homo sapiens
SDPHKSYLPV

SEQ ID NO: 131 - protein, 10 aa, Homo sapiens
QFQSPDKAET

SEQ ID NO: 132 - protein, 10 aa, Homo sapiens
QRGDPSVISV

SEQ ID NO: 133 - protein, 10 aa, Homo sapiens
NGTDFTFRSA

SEQ ID NO: 134 - protein, 10 aa, Homo sapiens
GVLLLVVDPC

SEQ ID NO: 135 - protein, 10 aa, Homo sapiens
SVPFRLTEKT

SEQ ID NO: 136 - protein, 10 aa, Homo sapiens
VFPLADVSRI

SEQ ID NO: 137 - protein, 10 aa, Homo sapiens
EEYLKTGIPP

SEQ ID NO: 138 - protein, 10 aa, Homo sapiens
RSIVLLSTRG

SEQ ID NO: 139 - protein, 10 aa, Homo sapiens
EIKQLNISHL

SEQ ID NO: 140 - protein, 10 aa, Homo sapiens
LVPLGLAKPA

SEQ ID NO: 141 - protein, 10 aa, Homo sapiens
HLYDKGSTIF

SEQ ID NO: 142 - protein, 10 aa, Homo sapiens
LGFSGNFKPS

SEQ ID NO: 143 - protein, 10 aa, Homo sapiens
WTKLFTSPAG

SEQ ID NO: 144 - protein, 10 aa, Homo sapiens
QGLGVLEQFI

SEQ ID NO: 145 - protein, 10 aa, Homo sapiens
PLQLDEYGCP

SEQ ID NO: 146 - protein, 10 aa, Homo sapiens
RATTVRRRDL

SEQ ID NO: 147 - protein, 10 aa, Homo sapiens
ELLKQASKAH

SEQ ID NO: 148 - protein, 1308 aa, Synthetic construct
DAAQPARRARRTKLGTSSKYAPDENCPDQNPRLRNWDPGQDSAKQVVIKEGDMLRLTSDATVHSIVIQDGGLLVFGDNKDGSRNITLRTHYILIQDGGALHIGAEKCRYKSKATITLYGKSDEGESMPTFGKKFIGVEAGGTLELHGARKASWTLLARTLNSSGLPFGSYTFEKDFSRGLNVRVIDQDTAKILESERFDTHEYRNESRRLQEFLRFQDPGRIVAIAVGDSAAKSLLQGTIQMIQERLGSELIQGLGYRQAWALVGVIDGGSTSCNESVRNYENHSSGGKALAQREFYTVDGQKFSVTAYSEWIEGVSLSGFRVEVVDGVKLNLLDDVSSWKPGDQIVVASTDYSMYQAEEFTLLPCSECSHFQVKVKETPQFLHMGEIIDGVDMRAEVGILTRNIVIQGEVEDSCYAENQCQFFDYDTFGGHIMIMKNFTSVHLSYVELKHMGQQQMGRYPVHFHLCGDVDYKGGYRHATFVDGLSIHHSFSRCITVHGTNGLLIKDTIGFDTLGHCFFLEDGIEQRNTLFHNLGLLTKPGTLLPTDRNNSMCTTMRDKVFGNYIPVPATDCMAVSTFWIAHPNNNLINNAAAGSQDAGIWYLFHKEPTGESSGLQLLAKPELTPLGIFYNNRVHSNFKAGLFIDKGVKTTNSSAADPREYLCLDNSARFRPHQDANPEKPRVAALIDRLIAFKNNDNGAWVRGGDIIVQNSAFADNGIGLTFASDGSFPSDEGSSQEVSESLFVGESRNYGFQGGQNKYVGTGGIDQKPRTLPRNRTFPIRGFQIYDGPIHLTRSTFKKYVPTPDRYSSAIGFLMKNSWQITPRNNISLVKFGPHVSLNVFFGKPGPWFEDCEMDGDKNSIFHDIDGSVTGYKDAYVGRMDNYLIRHPSCVNVSKWNAVICSGTYAQVYVQTWSTQNLSMTITRDEYPSNPMVLRGINQKAAFPQYQPVVMLEKGYTIHWNGPAPRTTFLYLVNFNKNDWIRVGLCYPSNTSFQVTFGYLQRQNGSLSKIEEYEPVHSLEELQRKQSERKFYFDSSTGLLFLYLKAKSHRHGHSYCSSQGCERVKIQAATDSKDISNCMAKAYPQYYRKPSVVKRMPAMLTGLCQGCGTRQVVFTSDPHKSYLPVQFQSPDKAETQRGDPSVISVNGTDFTFRSAGVLLLVVDPCSVPFRLTEKTVFPLADVSRIEEYLKTGIPPRSIVLLSTRGEIKQLNISHLLVPLGLAKPAHLYDKGSTIFLGFSGNFKPSWTKLFTSPAGQGLGVLEQFIPLQLDEYGCPRATTVRRRDLELLKQASKAHGSSENLYFQGSS

SEQ ID NO: 149 - DNA, 3975 bp, Synthetic construct
  source 1..3975: mol_type = other DNA
atggagacagacacactcctgctatgggtactgctgctctgggttccaggttccactggtgacgcggcccagccggccaggcgcgcgcgccgtacgaagcttggtacctcctcaaaatatgctccagatgaaaattgcccagatcaaaatcctcgtctcaggaattgggatccaggacaagattctgcaaagcaagttgttatcaaggagggagatatgctccgtctgacctcagacgccaccgtgcattctatagtcattcaggatggaggactgcttgtatttggggacaataaagatggatccagaaatattactttgaggactcattacatcctgatccaggatggtggggcgcttcatattggagcagaaaaatgccgctataaatccaaagcgacaattaccttgtatggcaagtcagatgaaggtgaaagtatgccaacatttggcaaaaagtttattggtgtggaagctggcgggacactggagttacatggggcacggaaggcatcgtggacgttgttggcaaggaccctgaattcctcaggcttgccctttgggtcctatacctttgaaaaggacttttcccggggcctcaatgtgagggtcattgaccaagacacggccaaaattttggaaagtgagagatttgatacccatgaataccgcaatgagagcaggcggcttcaggagtttctgagattccaggatccaggtcggattgttgccatagctgtcggggattcagccgctaaaagtctcttacaaggaaccatccagatgatccaggaacggttgggaagtgaactgatccaaggactgggctacaggcaagcttgggctttagttggtgtcattgatggtggaagcacttcttgcaatgaatccgtgagaaactatgaaaatcatagcagtggcgggaaggctcttgcccaaagagaattttatactgtggatggccagaagttctctgtgacagcttatagtgaatggattgaaggcgtttctctttcaggattccgggtagaggttgtagatggagtgaagctaaatttgctagatgatgttagtagttggaaacctggagaccagattgtggtcgcaagcacagactattccatgtaccaagcagaggagttcactcttcttccctgttctgaatgcagccattttcaggtcaaagtcaaagaaacccctcagttcctgcacatgggtgagatcatagacggtgtagacatgagagctgaggttggaattcttacccggaatattgtgatccaaggagaagtggaggactcatgctacgcagaaaatcagtgccaattttttgattatgatacctttgggggacacattatgataatgaaaaattttacttcagtccatctttcttatgtggaattgaaacacatgggtcagcagcagatggggcgataccctgttcattttcacctgtgtggtgacgtggattataaaggaggatacagacatgcaacatttgtggacggcctgtctattcatcacagcttctcaaggtgcatcactgtgcatgggacaaatggcttgctaataaaagacaccattgggtttgacacactaggtcattgtttctttttggaagatggtattgaacagaggaatactttgttccacaatctgggactcctcaccaagccgggtactctcctgcccaccgataggaacaactccatgtgtaccaccatgcgagataaagtgtttggaaattacattcctgtgcctgctactgactgtatggctgtttcaactttctggattgctcatcccaacaataatctgattaataatgcagctgcaggctcacaggatgctggaatatggtatttattccacaaggaaccaactggggaatccagtggattgcagctcttggcaaaaccagaactcactccattgggtatattttataacaacagggtccattcaaattttaaggctggcttatttattgacaaaggtgtcaaaacaaccaactctagtgctgctgacccaagggaatacctctgtttggacaatagtgcaagatttcgacctcatcaggatgcaaaccccgaaaaaccacgtgttgctgctctaattgacaggctcattgcttttaaaaataatgataatggagcttgggtcagaggaggagatattatcgttcaaaattcagcatttgcagataatggaataggactgacctttgccagtgatggaagcttcccaagtgatgaaggttccagccaagaggtatctgaatctctctttgttggggagagcaggaattacggctttcagggtggtcagaacaagtatgtaggcactggaggaatagaccagaagcctcgaacattacccaggaacaggacgttcccaattagaggctttcagatttatgatgggcccattcatctcacaaggagcactttcaaaaaatatgtgccaactccagataggtacagcagtgcaattggcttcctcatgaagaattcctggcagataacccccaggaataatatctccctcgtgaagtttggtccacatgtctctctgaatgtcttttttggaaagcctggtccctggtttgaagattgtgagatggatggtgataagaactccatattccatgacattgatggctctgtgacaggatacaaggatgcttatgtgggaagaatggacaactacctgatccgccatccaagctgtgtaaatgtgtctaagtggaatgcagtgatctgcagtgggacctatgcacaggtctatgtacagacatggagcactcagaatctttctatgaccattacacgagatgagtatccgtccaaccctatggtgctccgaggtattaatcagaaggctgcctttccacagtaccagcctgtcgtcatgctggagaagggttataccatccactggaatgggccggcaccacggactacatttctatacctcgtcaacttcaacaagaatgactggattcgagttggcctttgctatccatcaaacacaagttttcaagttacctttggctatttgcagcggcagaatggctcattatccaaaatcgaagaatatgagcctgtgcattcactggaagaactgcaaagaaagcaatccgagaggaaattctattttgactccagcacggggttactgtttttgtatctcaaagccaaaagccacaggcatggccacagttactgttcatctcagggatgtgaaagagtcaagatccaagcagccacagactcaaaggacatcagtaactgcatggccaaagcatacccacagtactacagaaagccgtcagtggtcaagcggatgccggccatgctcactggactctgtcaaggctgtggcactcggcaggtggtgtttactagtgatcctcataaaagttacctccctgtgcaattccagtcacctgataaagcagaaacccagcgtggagacccgtctgttatttctgtcaatggcactgactttaccttccgaagtgcaggcgtcctcctccttgttgtggatccgtgcagcgttccattccgcttgacggaaaaaacggtttttcctcttgctgatgtcagtcgcattgaagagtatttaaaaacaggcatccctccaaggtccattgttctgttgagcacaagaggagaaataaagcagttaaacatttcacacttactagtacctctgggattagccaaaccagctcatctttatgacaaagggagtaccatatttttgggattcagtggaaactttaaaccatcatggactaagctatttaccagtcctgctggacagggccttggggtgcttgaacaattcatacctttgcagctggacgaatatggttgtcccagagccaccactgtccgcagaagagacctggaactgctaaagcaagcttcaaaagcacatggttctagcgaaaacctgtattttcag

SEQ ID NO: 150 - protein, 1296 aa, Synthetic construct
DAAQPARRARRTKLGTSSKYAPDENCPDQNPRLRNWDPGQDSAKQVVIKEGDMLRLTSDATVHSIVIQDGGLLVFGDNKDGSRNITLRTHYILIQDGGALHIGAEKCRYKSKATITLYGKSDEGESMPTFGKKFIGVEAGGTLELHGARKASWTLLARTLNSSGLPFGSYTFEKDFSRGLNVRVIDQDTAKILESERFDTHEYRNESRRLQEFLRFQDPGRIVAIAVGDSAAKSLLQGTIQMIQERLGSELIQGLGYRQAWALVGVIDGGSTSCNESVRNYENHSSGGKALAQREFYTVDGQKFSVTAYSEWIEGVSLSGFRVEVVDGVKLNLLDDVSSWKPGDQIVVASTDYSMYQAEEFTLLPCSECSHFQVKVKETPQFLHMGEIIDGVDMRAEVGILTRNIVIQGEVEDSCYAENQCQFFDYDTFGGHIMIMKNFTSVHLSYVELKHMGQQQMGRYPVHFHLCGDVDYKGGYRHATFVDGLSIHHSFSRCITVHGTNGLLIKDTIGFDTLGHCFFLEDGIEQRNTLFHNLGLLTKPGTLLPTDRNNSMCTTMRDKVFGNYIPVPATDCMAVSTFWIAHPNNNLINNAAAGSQDAGIWYLFHKEPTGESSGLQLLAKPELTPLGIFYNNRVHSNFKAGLFIDKGVKTTNSSAADPREYLCLDNSARFRPHQDANPEKPRVAALIDRLIAFKNNDNGAWVRGGDIIVQNSAFADNGIGLTFASDGSFPSDEGSSQEVSESLFVGESRNYGFQGGQNKYVGTGGIDQKPRTLPRNRTFPIRGFQIYDGPIHLTRSTFKKYVPTPDRYSSAIGFLMKNSWQITPRNNISLVKFGPHVSLNVFFGKPGPWFEDCEMDGDKNSIFHDIDGSVTGYKDAYVGRMDNYLIRHPSCVNVSKWNAVICSGTYAQVYVQTWSTQNLSMTITRDEYPSNPMVLRGINQKAAFPQYQPVVMLEKGYTIHWNGPAPRTTFLYLVNFNKNDWIRVGLCYPSNTSFQVTFGYLQRQNGSLSKIEEYEPVHSLEELQRKQSERKFYFDSSTGLLFLYLKAKSHRHGHSYCSSQGCERVKIQAATDSKDISNCMAKAYPQYYRKPSVVKRMPAMLTGLCQGCGTRQVVFTSDPHKSYLPVQFQSPDKAETQRGDPSVISVNGTDFTFRSAGVLLLVVDPCSVPFRLTEKTVFPLADVSRIEEYLKTGIPPRSIVLLSTRGEIKQLNISHLLVPLGLAKPAHLYDKGSTIFLGFSGNFKPSWTKLFTSPAGQGLGVLEQFIPLQLDEYGCPRATTVRRRDLELLKQASKAH

SEQ ID NO: 151 - skipped

SEQ ID NO: 152 - protein, 1294 aa, Synthetic construct
GTSSKYAPDENCPDQNPRLRNWDPGQDSAKQVVIKEGDMLRLTSDATVHSIVIQDGGLLVFGDNKDGSRNITLRTHYILIQDGGALHIGAEKCRYKSKATITLYGKSDEGESMPTFGKKFIGVEAGGTLELHGARKASWTLLARTLNSSGLPFGSYTFEKDFSRGLNVRVIDQDTAKILESERFDTHEYRNESRRLQEFLRFQDPGRIVAIAVGDSAAKSLLQGTIQMIQERLGSELIQGLGYRQAWALVGVIDGGSTSCNESVRNYENHSSGGKALAQREFYTVDGQKFSVTAYSEWIEGVSLSGFRVEVVDGVKLNLLDDVSSWKPGDQIVVASTDYSMYQAEEFTLLPCSECSHFQVKVKETPQFLHMGEIIDGVDMRAEVGILTRNIVIQGEVEDSCYAENQCQFFDYDTFGGHIMIMKNFTSVHLSYVELKHMGQQQMGRYPVHFHLCGDVDYKGGYRHATFVDGLSIHHSFSRCITVHGTNGLLIKDTIGFDTLGHCFFLEDGIEQRNTLFHNLGLLTKPGTLLPTDRNNSMCTTMRDKVFGNYIPVPATDCMAVSTFWIAHPNNNLINNAAAGSQDAGIWYLFHKEPTGESSGLQLLAKPELTPLGIFYNNRVHSNFKAGLFIDKGVKTTNSSAADPREYLCLDNSARFRPHQDANPEKPRVAALIDRLIAFKNNDNGAWVRGGDIIVQNSAFADNGIGLTFASDGSFPSDEGSSQEVSESLFVGESRNYGFQGGQNKYVGTGGIDQKPRTLPRNRTFPIRGFQIYDGPIHLTRSTFKKYVPTPDRYSSAIGFLMKNSWQITPRNNISLVKFGPHVSLNVFFGKPGPWFEDCEMDGDKNSIFHDIDGSVTGYKDAYVGRMDNYLIRHPSCVNVSKWNAVICSGTYAQVYVQTWSTQNLSMTITRDEYPSNPMVLRGINQKAAFPQYQPVVMLEKGYTIHWNGPAPRTTFLYLVNFNKNDWIRVGLCYPSNTSFQVTFGYLQRQNGSLSKIEEYEPVHSLEELQRKQSERKFYFDSSTGLLFLYLKAKSHRHGHSYCSSQGCERVKIQAATDSKDISNCMAKAYPQYYRKPSVVKRMPAMLTGLCQGCGTRQVVFTSDPHKSYLPVQFQSPDKAETQRGDPSVISVNGTDFTFRSAGVLLLVVDPCSVPFRLTEKTVFPLADVSRIEEYLKTGIPPRSIVLLSTRGEIKQLNISHLLVPLGLAKPAHLYDKGSTIFLGFSGNFKPSWTKLFTSPAGQGLGVLEQFIPLQLDEYGCPRATTVRRRDLELLKQASKAHGSSENLYFQGSS

SEQ ID NO: 153 - DNA, 3915 bp, Synthetic construct
  source 1..3915: mol_type = other DNA
gacgcggcccagccggccaggcgcgcgcgccgtacgaagcttggtacctcctcaaaatatgctccagatgaaaattgcccagatcaaaatcctcgtctcaggaattgggatccaggacaagattctgcaaagcaagttgttatcaaggagggagatatgctccgtctgacctcagacgccaccgtgcattctatagtcattcaggatggaggactgcttgtatttggggacaataaagatggatccagaaatattactttgaggactcattacatcctgatccaggatggtggggcgcttcatattggagcagaaaaatgccgctataaatccaaagcgacaattaccttgtatggcaagtcagatgaaggtgaaagtatgccaacatttggcaaaaagtttattggtgtggaagctggcgggacactggagttacatggggcacggaaggcatcgtggacgttgttggcaaggaccctgaattcctcaggcttgccctttgggtcctatacctttgaaaaggacttttcccggggcctcaatgtgagggtcattgaccaagacacggccaaaattttggaaagtgagagatttgatacccatgaataccgcaatgagagcaggcggcttcaggagtttctgagattccaggatccaggtcggattgttgccatagctgtcggggattcagccgctaaaagtctcttacaaggaaccatccagatgatccaggaacggttgggaagtgaactgatccaaggactgggctacaggcaagcttgggctttagttggtgtcattgatggtggaagcacttcttgcaatgaatccgtgagaaactatgaaaatcatagcagtggcgggaaggctcttgcccaaagagaattttatactgtggatggccagaagttctctgtgacagcttatagtgaatggattgaaggcgtttctctttcaggattccgggtagaggttgtagatggagtgaagctaaatttgctagatgatgttagtagttggaaacctggagaccagattgtggtcgcaagcacagactattccatgtaccaagcagaggagttcactcttcttccctgttctgaatgcagccattttcaggtcaaagtcaaagaaacccctcagttcctgcacatgggtgagatcatagacggtgtagacatgagagctgaggttggaattcttacccggaatattgtgatccaaggagaagtggaggactcatgctacgcagaaaatcagtgccaattttttgattatgatacctttgggggacacattatgataatgaaaaattttacttcagtccatctttcttatgtggaattgaaacacatgggtcagcagcagatggggcgataccctgttcattttcacctgtgtggtgacgtggattataaaggaggatacagacatgcaacatttgtggacggcctgtctattcatcacagcttctcaaggtgcatcactgtgcatgggacaaatggcttgctaataaaagacaccattgggtttgacacactaggtcattgtttctttttggaagatggtattgaacagaggaatactttgttccacaatctgggactcctcaccaagccgggtactctcctgcccaccgataggaacaactccatgtgtaccaccatgcgagataaagtgtttggaaattacattcctgtgcctgctactgactgtatggctgtttcaactttctggattgctcatcccaacaataatctgattaataatgcagctgcaggctcacaggatgctggaatatggtatttattccacaaggaaccaactggggaatccagtggattgcagctcttggcaaaaccagaactcactccattgggtatattttataacaacagggtccattcaaattttaaggctggcttatttattgacaaaggtgtcaaaacaaccaactctagtgctgctgacccaagggaatacctctgtttggacaatagtgcaagatttcgacctcatcaggatgcaaaccccgaaaaaccacgtgttgctgctctaattgacaggctcattgcttttaaaaataatgataatggagcttgggtcagaggaggagatattatcgttcaaaattcagcatttgcagataatggaataggactgacctttgccagtgatggaagcttcccaagtgatgaaggttccagccaagaggtatctgaatctctctttgttggggagagcaggaattacggctttcagggtggtcagaacaagtatgtaggcactggaggaatagaccagaagcctcgaacattacccaggaacaggacgttcccaattagaggctttcagatttatgatgggcccattcatctcacaaggagcactttcaaaaaatatgtgccaactccagataggtacagcagtgcaattggcttcctcatgaagaattcctggcagataacccccaggaataatatctccctcgtgaagtttggtccacatgtctctctgaatgtcttttttggaaagcctggtccctggtttgaagattgtgagatggatggtgataagaactccatattccatgacattgatggctctgtgacaggatacaaggatgcttatgtgggaagaatggacaactacctgatccgccatccaagctgtgtaaatgtgtctaagtggaatgcagtgatctgcagtgggacctatgcacaggtctatgtacagacatggagcactcagaatctttctatgaccattacacgagatgagtatccgtccaaccctatggtgctccgaggtattaatcagaaggctgcctttccacagtaccagcctgtcgtcatgctggagaagggttataccatccactggaatgggccggcaccacggactacatttctatacctcgtcaacttcaacaagaatgactggattcgagttggcctttgctatccatcaaacacaagttttcaagttacctttggctatttgcagcggcagaatggctcattatccaaaatcgaagaatatgagcctgtgcattcactggaagaactgcaaagaaagcaatccgagaggaaattctattttgactccagcacggggttactgtttttgtatctcaaagccaaaagccacaggcatggccacagttactgttcatctcagggatgtgaaagagtcaagatccaagcagccacagactcaaaggacatcagtaactgcatggccaaagcatacccacagtactacagaaagccgtcagtggtcaagcggatgccggccatgctcactggactctgtcaaggctgtggcactcggcaggtggtgtttactagtgatcctcataaaagttacctccctgtgcaattccagtcacctgataaagcagaaacccagcgtggagacccgtctgttatttctgtcaatggcactgactttaccttccgaagtgcaggcgtcctcctccttgttgtggatccgtgcagcgttccattccgcttgacggaaaaaacggtttttcctcttgctgatgtcagtcgcattgaagagtatttaaaaacaggcatccctccaaggtccattgttctgttgagcacaagaggagaaataaagcagttaaacatttcacacttactagtacctctgggattagccaaaccagctcatctttatgacaaagggagtaccatatttttgggattcagtggaaactttaaaccatcatggactaagctatttaccagtcctgctggacagggccttggggtgcttgaacaattcatacctttgcagctggacgaatatggttgtcccagagccaccactgtccgcagaagagacctggaactgctaaagcaagcttcaaaagcacatggttctagcgaaaacctgtattttcag

(19) United States
(12) Patent Application Publication
(10) Pub. No.: US 2025/0205315 A1
(43) Pub. Date: Jun. 26, 2025

(54) RECOMBINANT TMEM2 EXTRACELLULAR DOMAIN AND USES THEREOF

(71) Applicant: Sanford Burnham Prebys Medical Discovery Institute, La Jolla, CA (US)

(72) Inventors: Yu YAMAGUCHI, San Diego, CA (US); Fumitoshi IRIE, San Diego, CA (US); Michael R. JACKSON, Del Mar, CA (US); Eduard SERGIENKO, San Diego, CA (US); Andrey BOBKOV, Escondido, CA (US)

(21) Appl. No.: 18/984,233

(22) Filed: Dec. 17, 2024

Related U.S. Application Data
(60) Provisional application No. 63/614,379, filed on Dec. 22, 2023.

(51) Int. Cl. A61K 38/47; C07K 14/705; C12N 9/26

(52) U.S. Cl. CPC A61K 38/47; C07K 14/705; C12N 9/2474; C12Y 302/01035

(57) ABSTRACT

Described herein is a recombinant TMEM2 extracellular domain which can be used as an adjuvant to increase the injection volume and dispersion of compositions containing one or more therapeutic agents. Methods of preparing recombinant TMEM2 extracellular domain protein and their use in combination with one or more therapeutic agents as medicaments are provided.

### CROSS-REFERENCE

Any and all priority claims identified in the Application Data sheet, or any correction thereto, are hereby incorporated by reference under 37 CFR 1.57. For example, this application claims the benefit of U.S. Provisional Application No. 63/614,379 filed on Dec. 22, 2023, which is incorporated herein by reference in its entirety.

### SEQUENCE LISTING

The instant application contains a Sequence Listing which has been submitted electronically in XML format and is hereby incorporated by reference in its entirety. Said XML copy, created on Dec. 17, 2024, is named 42256-621_201.xml and is 156,425 bytes in size.

### BACKGROUND

### Field

Aspects of the present disclosure relate to recombinant TMEM2 extracellular domain as an adjuvant to increase the injection volume and dispersion of compositions containing one or more therapeutic agents. Methods of making recombinant TMEM2 extracellular domain and methods of utilization are provided.

### Description of the Related Technology

Over the last decade, considerable research has shown that the HYAL family of hyaluronidases plays significant roles in HA catabolism. Nonetheless, the identity of a hyaluronidase responsible for the initial step of HA cleavage on the cell surface remains elusive, as biochemical and enzymological properties of HYAL proteins are not entirely consistent with those expected of cell surface hyaluronidases.

Previously identified hyaluronidases such as PH-20/SPAM1, HYAL1, and HYAL2 have optimal activity below pH 5.5.

### SUMMARY

In some aspects, disclosed herein is a recombinant protein, comprising a human TMEM2 extracellular domain or a fragment thereof, wherein the recombinant protein has hyaluronidase catalytic activity.

In some embodiments, the recombinant protein disclosed herein comprises a human TMEM2 extracellular domain that comprises one or more amino acid residues from 1000-1280 residues.

In some embodiments, the recombinant protein disclosed herein comprises a human TMEM2 extracellular domain that comprises a complete sequence of amino acids set forth in SEQ ID NO. 20 (SSKYAPDENC) and/or SEQ ID NO. 90 (NGIGLTFASD).

In some embodiments, the recombinant protein disclosed herein further comprises at least one N-linked sugar moiety.

In some embodiments, the recombinant protein disclosed herein further comprises a C-terminal linked sequence of amino acids or N-terminal linked sequence of amino acids.

In some embodiments, the recombinant protein disclosed herein comprises a N-terminal linked sequence of amino acids that comprises a complete sequence of amino acids set forth in SEQ ID NO. 8 (DAAQPARRARRTKLGT).

In some embodiments, the recombinant protein disclosed herein further comprises at least one linked polymer, wherein the polymer is PEG or dextran.

In some embodiments, the recombinant protein disclosed herein comprises a complete sequence of amino acids set forth in SEQ ID NO. 1 or at least 90% amino acid sequence identity with a sequence of amino acids set forth in SEQ ID NO. 1.

In some embodiments, the recombinant protein disclosed herein comprises a complete sequence of amino acids set forth in SEQ ID NO. 1.

In some aspects, disclosed herein is a recombinant protein, comprising at least 1279 amino acid residues of human TMEM2 extracellular domain, wherein the recombinant protein does not comprise the complete sequence of amino acids set forth in SEQ ID NO. 1, or the recombinant protein has at least 95% amino acid sequence identity with a sequence of amino acids set forth in SEQ ID NO. 1, and the recombinant protein comprises a complete sequence of amino acids having at least 90% amino acid sequence identity with a sequence of amino acids set forth in SEQ ID NO. 20 (SSKYAPDENC) and/or SEQ ID NO. 90 (NGIGLTFASD).

In some embodiments, the recombinant protein disclosed herein comprises at least 1200, 1220, 1240, 1260, or 1278 amino acid residues of human TMEM2 extracellular domain, wherein the recombinant protein does not comprise SEQ ID NO. 7 (APLVHHHHHHALDENLYFQGALA).

In some embodiments, the recombinant protein disclosed herein consists of a contiguous sequence of amino acid residues contained within SEQ ID NO. 1 that includes at least amino acids 1-250, 250-500, 501-750, 751-1000 or 1001-1280 of SEQ ID NO. 1, or the recombinant protein has at least 95% amino acid sequence identity with a sequence of amino acids set forth in SEQ ID NO. 1.

In some embodiments, the recombinant protein disclosed herein consists of a contiguous sequence of amino acid residues contained within SEQ ID NO. 1 that includes at least amino acids 1-250, 250-500, 501-750, 751-1000 or 1001-1280 of SEQ ID NO. 1.

In some embodiments, the recombinant protein disclosed herein has at least 95%, 96% or 97% amino acid sequence identity with a sequence of amino acids set forth in SEQ ID NO. 1.

In some embodiments, the recombinant protein disclosed herein has less than 100% or less than 98% amino acid sequence identity with a sequence of amino acids set forth in SEQ ID NO. 1.

In some embodiments, recombinant protein disclosed herein comprises a sequence of amino acids set forth as amino acids 1-200, 201-400, 401-600, 601-800, 801-1000, or 1001-1280 of SEQ ID NO. 1; or (ii) the recombinant protein disclosed herein contains amino acid substitutions in the sequence of amino acids set forth as amino acids 1-200, 201-400, 401-600, 601-800, 801-1000, or 1001-1280 of SEQ ID NO. 1, whereby the amino acid-substituted recombinant protein consists of a sequence of amino acids that has at least 95% amino acid sequence identity with the sequence of amino acids set forth as amino acids 1-400, 401-800, or 801-1280 of SEQ ID NO. 1.

In some embodiments, the recombinant protein disclosed herein comprises at least two, three, four, five, six, seven, eight, nine, ten, 11, 12, 13, 14, 15, 16, 17, 18, or 19 sequences of amino acids set forth as amino acids 1-50, 51-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100, 1101-1150, 1151-1200, or 1201-1280 of SEQ ID NO. 1.

In some embodiments, the recombinant protein disclosed herein comprises less than 24, or 25 sequences of amino acids, whereby the recombinant protein consists of a sequence of amino acids that has at least 95%, 96%, 97% or 98% amino acid sequence identity with the sequence of amino acids set forth in SEQ ID NO. 1.

In some embodiments, the recombinant protein disclosed herein has hyaluronidase catalytic activity of at least 50%, at least 60%, at least 70%, at least 80% or at least 90% of TMEM2 extracellular domain.

In some embodiments, the recombinant protein disclosed herein comprises a complete sequence of amino acids having 90% amino acid sequence identity with a sequence of amino acids set forth in SEQ ID NO. 90 (NGIGLTFASD) where D of SEQ ID NO. 90 is replaced with E.

In some aspects, disclosed herein is a recombinant protein, comprising a human TMEM2 extracellular domain or a fragment thereof, wherein the recombinant protein comprises an amino acid sequence as set forth in SEQ ID NO. 10, SEQ ID NO. 148, SEQ ID NO. 150, or SEQ ID NO. 152.

In some embodiments, the recombinant protein disclosed herein has hyaluronidase catalytic activity of at least 50%, at least 60%, at least 70%, at least 80% or at least 90% of TMEM2 extracellular domain.

In some embodiments, the recombinant protein disclosed herein comprises a complete sequence of amino acids having 90% amino acid sequence identity with a sequence of amino acids set forth in SEQ ID NO. 90 (NGIGLTFASD) where D of SEQ ID NO. 90 is replaced with E.

In some embodiments, the recombinant protein disclosed herein comprises a mutation.

In some embodiments, the mutation is selected from a deletion, insertion, or substitution.

In some embodiments, the mutation is a substitution.

In some embodiments, wherein the mutation is a point mutation at position 813 of SEQ ID NO. 1.

In some embodiments, the amino acid residue D at position 813 of SEQ ID NO. 1 is substituted with amino acid residue N.

In another aspect, disclosed herein is a protein that is a mutein of a polypeptide of 1000 to 1280 amino acid residues of TMEM2 extracellular domain, wherein: up to about 50% of the amino acids are replaced with another amino acid; and the resulting polypeptide is a single chain or two chain polypeptide that has catalytic activity of at least 10% of 1000 to 1280 amino acid residues of TMEM2 extracellular domain.

In some embodiments, in the protein disclosed herein about 10% of the amino acids are replaced with another amino acid.

In some embodiments, in the protein disclosed herein the resulting polypeptide is a single chain or two chain polypeptide and has catalytic activity of at least 50%, at least 60%, at least 70%, at least 80% or at least 90% of TMEM2 extracellular domain.

In another aspect, disclosed herein is a preparation comprising a recombinant protein disclosed herein or a protein disclosed herein.

In some embodiments, the preparation disclosed herein further comprises a buffer.

In some embodiments, the preparation disclosed herein has >95% purity as assessed by Coomassie stained SDS PAGE.

In another aspect, provided herein is a pharmaceutical composition, comprising a recombinant protein disclosed herein or a protein disclosed herein.

In another aspect, disclosed herein is a pharmaceutical composition, comprising a recombinant protein disclosed herein or a protein disclosed herein and a pharmaceutically acceptable excipient.

In some embodiments, the pharmaceutical composition disclosed herein further comprises a pharmaceutically active agent.

In some embodiments, the pharmaceutical composition disclosed herein comprises a the pharmaceutically active agent selected from the group consisting of an antibody, an antibody fragment, a small molecule inhibitor, a vaccine, and an immunoglobulin.

In some embodiments, the pharmaceutical composition disclosed herein further comprises a stabilizing solution.

In some embodiments, the stabilizing solution further comprises a carrier selected from the group comprising albumin, detergent, or a surfactant.

In another aspect, disclosed herein is a nucleic acid molecule, comprising a sequence of nucleotides that encodes a recombinant protein disclosed herein or a protein disclosed herein.

In another aspect, disclosed herein is a nucleic acid molecule, comprising a sequence of nucleotides selected from the group consisting of: (a) a sequence of nucleotides set forth in any of SEQ ID Nos. 2, 11, 149, or 153; (b) a sequence that hybridizes under high stringency to at least about 60%, 70%, 80%, 90% or 95% of the full-length to the sequence of nucleotides set forth in SEQ ID NO. 4; (c) a sequence of nucleotides that encodes an TMEM2 extracellular domain or a catalytically active portion thereof that includes a sequence of nucleotides having at least about 60%, 70%, 80%, 90% or 95% sequence identity the sequence set forth in SEQ ID Nos. 2, 4, 11, 149, or 153; (d) a sequence that is a splice variant of (a), (b) or (c); and (e) a sequence comprising degenerate codons of (a), (b), (c) or (d).

In another aspect, disclosed herein is a vector comprising a nucleic acid molecule disclosed herein.

In some embodiments, the vector is an expression vector.

In some embodiments, the vector is a eukaryotic vector.

In some embodiments, the vector includes a sequence of nucleotides that directs secretion of any polypeptide encoded by a sequence of nucleotides operatively linked thereto.

In some embodiments, the vector is a Pichia vector or an E. coli vector.

In another aspect, disclosed herein is a cell comprising a vector disclosed herein.

In some embodiments, the cell is a prokaryotic cell.

In some embodiments, the cell is a is a eukaryotic cell.

In some embodiments, the cell is selected from among a bacterial cell, a yeast cell, a plant cell, an insect cell and an animal cell.

In some embodiments, the cell is a mammalian cell.

In another aspect, disclosed herein is a pharmaceutical composition comprising a recombinant protein disclosed herein, a protein disclosed herein, a preparation disclosed herein, a nucleic acid disclosed herein, a vector disclosed herein, or a cell disclosed herein.

In another aspect, disclosed herein is a method for generating a recombinant protein disclosed herein or a protein disclosed herein, comprising introduction of a nucleic acid as described in any of SEQ ID NOs. 2, 4, 11, 149, or 153 optionally operably linked to a suitable promoter into a eukaryotic cell.

In some embodiments, the eukaryotic cell is mammalian.

In some embodiments, the eukaryotic cell is an insect cell.

In some embodiments, the eukaryotic cell is a yeast cell.

In some embodiments, eukaryotic cell is a plant cell.

In another aspect, disclosed herein is a method for increasing the diffusion of a therapeutic substance in a subject, comprising: administering to a subject a recombinant protein disclosed herein or a protein disclosed herein; and a therapeutic substance, whereby the diffusion of the therapeutic substance is increased.

In some embodiments, in the method disclosed herein a recombinant protein disclosed herein or a protein disclosed herein is mixed with the therapeutic substance prior to administration.

In another aspect, disclosed herein is a method for increasing the absorption of a therapeutic substance in a subject, comprising: administering to a subject a recombinant protein disclosed herein or a protein disclosed herein and a therapeutic substance, whereby the absorption of the therapeutic substance is increased.

In some embodiments, in the method disclosed herein a recombinant protein disclosed herein or a protein disclosed herein is mixed with the therapeutic substance prior to administration.

In another aspect, disclosed herein is a kit, comprising: a) a recombinant protein disclosed herein or a protein disclosed herein at a dose between 0.1 and 1500 Units/mL in an acceptable carrier; (b) at least one therapeutic substance in a pharmaceutically acceptable carrier; and (c) optionally, instructions for delivering therapeutic substances.

In some embodiments, in the kit disclosed herein the recombinant protein disclosed herein or the protein disclosed herein are provided as a mixture.

In another aspect, disclosed herein is an injectable depot formulation comprising i) a recombinant protein disclosed herein or a protein disclosed herein; and ii) a therapeutic substance.

In some embodiments, in the injectable depot formulation disclosed herein, the therapeutic substance is in the form of particles having an average particle size of less than about 60000 nm, 50000 nm, 40000 nm, 20000 nm, 10000 nm, 2000 nm, 1500 nm, 1000 nm, 500 nm, 250 nm, 100 nm or 50 nm, or an average particle size within a range defined by any of the preceding values.

In some embodiments, in the injectable depot formulation disclosed herein the particles are resistant to deformation or amenable to deformation at physiological temperatures.

In another aspect, disclosed herein is a method dissolving hyaluronic acid fillers in a subject, the method comprising: administering to the subject a recombinant protein disclosed herein, a protein disclosed herein, a preparation disclosed herein, a pharmaceutical composition disclosed herein, a nucleic acid disclosed herein, a vector disclosed herein, a cell disclosed herein, or an injectable depot formulation disclosed herein, whereby the dissolution of hyaluronic fillers is induced.

In another aspect, disclosed herein is a method for removing small areas of excess hyaluronic acid fillers in a subject, the method comprising: administering to a subject with hyaluronic acid fillers a recombinant protein disclosed herein, a protein disclosed herein, a preparation disclosed herein, a pharmaceutical composition disclosed herein, or an injectable depot formulation disclosed herein, whereby the dissolution of hyaluronic fillers is induced.

In another aspect, disclosed herein is a method for complete removal of hyaluronic acid fillers in a subject, the method comprising: administering to a subject with hyaluronic acid fillers a recombinant protein disclosed herein, a protein disclosed herein, a preparation disclosed herein, a pharmaceutical composition disclosed herein, or an injectable depot formulation disclosed herein, whereby the dissolution of hyaluronic fillers is induced.

In some embodiments, the subject received at least one hyaluronic acid filler administration prior to administering the recombinant TMEM2 protein or fragment thereof, preparation, pharmaceutical composition, or injectable depot formulation.

In some embodiments, the subject received more than one hyaluronic acid filler administration prior to administering the recombinant TMEM2 protein or fragment thereof, nucleotide, vector, cell, preparation, pharmaceutical composition, or injectable depot formulation.

In some embodiments, the subject received the hyaluronic acid filler administration at least one month before the administration of the recombinant TMEM2 protein or fragment thereof, preparation, pharmaceutical composition, or injectable depot formulation.

In some embodiments, the subject received an excess hyaluronic acid filler administration.

In some embodiments, in a recombinant protein disclosed herein the N-terminal linked sequence of amino acids does not comprise a complete sequence of amino acids set forth in SEQ ID NO. 8 (DAAQPARRARRTKLGT).

In some embodiments, the therapeutic agent is a therapeutic agent selected from one or more of an antianginal agent, an antiarrhythmic, an antiasthmatic agent, an antibiotic agent, an antidiabetic agent, an antifungal agent, an antihistamine, an antihypertensive agent, an antiparasitic agent, an antineoplastic agent, an antiviral agent, an ontological agent, a cardiac glycoside, a hormone, an immunomodulator, a monoclonal antibody, a neurotransmitter, a sedative, a vaccine, a vasopressor, an anesthetic agent, an amide anesthetic agent, a corticosteroid, a tricyclic antidepressant, a tetracyclic antidepressant, a serotonin reuptake inhibitor, a steroid receptor modulator, an antipsychotic drug, an antiprotozoal drug, an opioid, an antiproliferative agent, a salicylanilides, an anthelminthic drug, a vinca alkaloids, an anti-inflammatory agent, an anti-depressant, a prostaglandin, a phosphodiesterase IV inhibitor, a retinoid, a steroids, a β-adrenergic receptor ligand, an anti-mitotic agent, a microtubule inhibitor, a microtubule-stabilizing agent, a serotonin norepinephrine reuptake inhibitor, a noradrenaline reuptake inhibitor, a non-steroidal immunophilin-dependent immunosuppressant, a non-steroidal immunophilin-dependent immunosuppressant enhancer, an antimalarial agent, an analgesic agent, an immunosuppressant, an expectorant, a sulfa drug, a cardiovascular drug, a central nervous system (CNS) depressant, an H2-blocker, an anti-platelet drug, an anticonvulsant agent, an alpha blocker, a beta-blocker, a cholinesterase inhibitor, a calcium channel blocker, an H1-receptor antagonist, or a proteinaceous material.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Some novel features of the methods and compositions disclosed herein are set forth in the present disclosure. These and other objects and features of the embodiments will become more fully apparent in the following descriptions and appended claims taken in conjunction with the following drawings:

FIG. 1 shows that mouse TMEM2 extracellular domain is an effective spreading agent similar to the recombinant PH-20 protein.

FIGS. 2A-2B compares models of human TMEM2 generated using Alphafold2 and cryo-EM. FIG. 2A shows the model of human TMEM2 generated using Alphafold2. FIG. 2B shows the model of human TMEM2 generated using cryo-EM.

FIG. 3 shows the localization of certain acidic amino acid residues on a cryo-EM generated model of human TMEM2.

FIG. 4 shows a rotated version of the human TMEM2 model generated by cryo-EM that also identifies the localization of certain acidic amino acid residues.

FIGS. 5A-5B shows docking localization for the number one scoring candidate on a cryo-EM generated model of human TMEM2. FIG. 5A shows docking localization for the number one scoring candidate on a cryo-EM generated model of human TMEM2. FIG. 5B shows docking localization for the number one scoring candidate on a cryo-EM generated model of human TMEM2 rotated by ninety degrees.

FIG. 6 shows a computational model showing the interaction between human TMEM2 and hyaluronan.

FIG. 7 provides a summary of hyaluronidase assay experiments for TMEM2 performed using samples from the P2 membrane fraction after centrifugation.

FIG. 8 shows results from hyaluronidase assay and protein expression experiments using human wild type TMEM2 and various TMEM2 mutants.

FIG. 9 shows results from hyaluronidase assay and protein expression experiments using mouse wild type TMEM2 and various TMEM2 mutants.

FIGS. 10A-10B show quantification of hyaluronidase activity from cells with various amino acid mutations. FIG. 10A shows the effects of amino acid residue mutations on human TMEM2 hyaluronidase activity. FIG. 10B shows the effects of amino acid residue mutations on mouse TMEM2 hyaluronidase activity.

FIG. 11 provides a summary of hyaluronidase assay experiments involving the extracellular domain of TMEM2.

FIG. 12 shows quantification of fluorescent HA size from a TMEM2 extracellular domain hyaluronidase activity assay experiment using cells transfected with wildtype and D813N mutant TMEM2 extracellular domain.

FIG. 13A-13B show the effects of a D813N mutation on TMEM2-dependent cellular function. FIG. 13A shows in situ degradation of HA in control and mutant cells. FIG. 13B shows HA-related cell migration in control and mutant cells.

### DETAILED DESCRIPTION

While various embodiments have been shown and described herein, it will be understood by those skilled in the art that such embodiments are provided by way of example only. Numerous variations, changes, and substitutions may occur to those skilled in the art without departing from the embodiments. It should be understood that various alternatives to the embodiments described herein may be employed.

Hyaluronidase, such as the recombinant formulation of hyaluronidase marketed under HYLENEX™, a recombinant human PH20 fragment, has received FDA market approval as of December 2005 as an adjuvant to increase the absorption and dispersion of injected drugs. The enzyme acts by cleaving hyaluronan present in the interstitial collagenous matrix and results in less extracellular matrix resistance. Following cleavage, the effects of hyaluronidase are completely reversible within 24 h of injection. Hyaluronidase has been evaluated for efficacy of dispersion with particles from 20 nm to 1 μm. It has been demonstrated that under some conditions PH20 degrades hyaluronan to afford products with weight-averages sizes of 63.9±0.5 kDa with 98% of the products ranging from 5.5 to 227 kDa. It is postulated that the products are sufficient to inhibit remyelination in vivo. Also, it has been demonstrated that PH20 degrades chondroitin sulfate. In contrast, TMEM2 degrades high molecular weight hyaluronan (HA) into ˜5 KDa fragments and does not degrade chondroitin sulfate.

In one aspect, the disclosure provides a recombinant protein comprising a TMEM2 protein or fragment thereof. In some embodiments, the TMEM2 protein or fragment thereof is a mammalian TMEM2 protein or fragment thereof.

In some embodiments, the TMEM2 protein or fragment thereof is a human TMEM2 protein or fragment thereof. In some embodiments, the human TMEM2 protein comprises an amino acid sequence as set forth in NCBI Accession AAI40774.1, NP_001129292.1, NP_037522.1, NP_001336713.1, Q9UHN6.1, AAF21348.1, XP_005251926.1, EAW62520.1, XP_054218564.1, AAI46781.1, EAW62517.1, or Q8WUJ3.2. In some embodiments the human TMEM2 protein is encoded by a polynucleotide comprising a nucleotide sequence set forth in NCBI accession no. NG 053053. In some embodiments the human TMEM2 protein is encoded by a polynucleotide comprising the nucleotide sequence set forth in SEQ ID NO. 2.

(SEQ ID NO. 3)
MYATDSRGHSPAFLQPQNGNSRHPSGYVPGKVVPLRPPPPPKSQASAKFTSIR
REDRATFAFSPEEQQAQRESQKQKRHKNTFICFAITSFSFFIALAIILGISSKYAPDENCP
DQNPRLRNWDPGQDSAKQVVIKEGDMLRLTSDATVHSIVIQDGGLLVFGDNKDGSR
NITLRTHYILIQDGGALHIGAEKCRYKSKATITLYGKSDEGESMPTFGKKFIGVEAGGT
LELHGARKASWTLLARTLNSSGLPFGSYTFEKDFSRGLNVRVIDQDTAKILESERFDT
HEYRNESRRLQEFLRFQDPGRIVAIAVGDSAAKSLLQGTIQMIQERLGSELIQGLGYR
QAWALVGVIDGGSTSCNESVRNYENHSSGGKALAQREFYTVDGQKFSVTAYSEWIE
GVSLSGFRVEVVDGVKLNLLDDVSSWKPGDQIVVASTDYSMYQAEEFTLLPCSECSH
FQVKVKETPQFLHMGEIIDGVDMRAEVGILTRNIVIQGEVEDSCYAENQCQFFDYDTF
GGHIMIMKNFTSVHLSYVELKHMGQQQMGRYPVHFHLCGDVDYKGGYRHATFVDG
LSIHHSFSRCITVHGTNGLLIKDTIGFDTLGHCFFLEDGIEQRNTLFHNLGLLTKPGTLL
PTDRNNSMCTTMRDKVFGNYIPVPATDCMAVSTFWIAHPNNNLINNAAAGSQDAGI
WYLFHKEPTGESSGLQLLAKPELTPLGIFYNNRVHSNFKAGLFIDKGVKTTNSSAADP
REYLCLDNSARFRPHQDANPEKPRVAALIDRLIAFKNNDNGAWVRGGDIIVQNSAFA
DNGIGLTFASDGSFPSDEGSSQEVSESLFVGESRNYGFQGGQNKYVGTGGIDQKPRTL
PRNRTFPIRGFQIYDGPIHLTRSTFKKYVPTPDRYSSAIGFLMKNSWQITPRNNISLVKF
GPHVSLNVFFGKPGPWFEDCEMDGDKNSIFHDIDGSVTGYKDAYVGRMDNYLIRHP
SCVNVSKWNAVICSGTYAQVYVQTWSTQNLSMTITRDEYPSNPMVLRGINQKAAFP
QYQPVVMLEKGYTIHWNGPAPRTTFLYLVNFNKNDWIRVGLCYPSNTSFQVTFGYL
QRQNGSLSKIEEYEPVHSLEELQRKQSERKFYFDSSTGLLFLYLKAKSHRHGHSYCSS
QGCERVKIQAATDSKDISNCMAKAYPQYYRKPSVVKRMPAMLTGLCQGCGTRQVV
FTSDPHKSYLPVQFQSPDKAETQRGDPSVISVNGTDFTFRSAGVLLLVVDPCSVPFRLT
EKTVFPLADVSRIEEYLKTGIPPRSIVLLSTRGEIKQLNISHLLVPLGLAKPAHLYDKGS
TIFLGFSGNFKPSWTKLFTSPAGQGLGVLEQFIPLQLDEYGCPRATTVRRRDLELLKQ
ASKAH

In some embodiments, the TMEM2 protein or fragment thereof is a mouse TMEM2 protein or fragment thereof. In some embodiments, the TMEM2 protein comprises an amino acid sequence selected from any of SEQ ID NOs. 1, 3, 10, 20-148, 150, or 152. In some embodiments, the TMEM2 protein does not comprise an amino acid sequence selected from one or more of SEQ ID NOs. 5, 6, or 9. In some embodiments, the TMEM2 protein comprises an amino acid sequence as set forth in Table 1.

In some embodiments, the TMEM2 protein is a TMEM2 extracellular domain, e.g., human TMEM2 extracellular domain, e.g., comprising residues 104-1383 of human TMEM2. In some embodiments, the human extracellular domain comprises an amino acid sequence as set forth in SEQ ID NO. 1.

(SEQ ID NO. 1)
SSKYAPDENCPDQNPRLRNWDPGQDSAKQVVIKEGDMLRLTSDATVHSIVIQ
DGGLLVFGDNKDGSRNITLRTHYILIQDGGALHIGAEKCRYKSKATITLYGKSDEGES
MPTFGKKFIGVEAGGTLELHGARKASWTLLARTLNSSGLPFGSYTFEKDFSRGLNVR
VIDQDTAKILESERFDTHEYRNESRRLQEFLRFQDPGRIVAIAVGDSAAKSLLQGTIQM
IQERLGSELIQGLGYRQAWALVGVIDGGSTSCNESVRNYENHSSGGKALAQREFYTV
DGQKFSVTAYSEWIEGVSLSGFRVEVVDGVKLNLLDDVSSWKPGDQIVVASTDYSM
YQAEEFTLLPCSECSHFQVKVKETPQFLHMGEIIDGVDMRAEVGILTRNIVIQGEVEDS
CYAENQCQFFDYDTFGGHIMIMKNFTSVHLSYVELKHMGQQQMGRYPVHFHLCGD
VDYKGGYRHATFVDGLSIHHSFSRCITVHGTNGLLIKDTIGFDTLGHCFFLEDGIEQRN
TLFHNLGLLTKPGTLLPTDRNNSMCTTMRDKVFGNYIPVPATDCMAVSTFWIAHPNN
NLINNAAAGSQDAGIWYLFHKEPTGESSGLQLLAKPELTPLGIFYNNRVHSNFKAGLF
IDKGVKTTNSSAADPREYLCLDNSARFRPHQDANPEKPRVAALIDRLIAFKNNDNGA
WVRGGDIIVQNSAFADNGIGLTFASDGSFPSDEGSSQEVSESLFVGESRNYGFQGGQN
KYVGTGGIDQKPRTLPRNRTFPIRGFQIYDGPIHLTRSTFKKYVPTPDRYSSAIGFLMK
NSWQITPRNNISLVKFGPHVSLNVFFGKPGPWFEDCEMDGDKNSIFHDIDGSVTGYK
DAYVGRMDNYLIRHPSCVNVSKWNAVICSGTYAQVYVQTWSTQNLSMTITRDEYPS
NPMVLRGINQKAAFPQYQPVVMLEKGYTIHWNGPAPRTTFLYLVNFNKNDWIRVGL
CYPSNTSFQVTFGYLQRQNGSLSKIEEYEPVHSLEELQRKQSERKFYFDSSTGLLFLYL
KAKSHRHGHSYCSSQGCERVKIQAATDSKDISNCMAKAYPQYYRKPSVVKRMPAML
TGLCQGCGTRQVVFTSDPHKSYLPVQFQSPDKAETQRGDPSVISVNGTDFTFRSAGVL
LLVVDPCSVPFRLTEKTVFPLADVSRIEEYLKTGIPPRSIVLLSTRGEIKQLNISHLLVPL
GLAKPAHLYDKGSTIFLGFSGNFKPSWTKLFTSPAGQGLGVLEQFIPLQLDEYGCPRA
TTVRRRDLELLKQASKAH

In some embodiments, the recombinant protein comprises a human TMEM2 extracellular domain fragment, e.g., one or more residues selected from amino acid residues 104-1383 of human TMEM2. In some embodiments, the human TMEM2 extracellular domain fragment comprises between 1000 and 1118 residues. In some embodiments, the recombinant protein comprises at least 1000 residues. In some embodiments, the recombinant protein comprises an amino acid sequence as set forth in SEQ ID NO. 10, 148, 150, or 152.

(SEQ ID NO. 10)
DAAQPARRARRTKLGTSSKYAPDENCPDQNPRLRNWDPGQDSAKQVVIKEG
DMLRLTSDATVHSIVIQDGGLLVFGDNKDGSRNITLRTHYILIQDGGALHIGAEKCRY
KSKATITLYGKSDEGESMPTFGKKFIGVEAGGTLELHGARKASWTLLARTLNSSGLPF
GSYTFEKDFSRGLNVRVIDQDTAKILESERFDTHEYRNESRRLQEFLRFQDPGRIVAIA
VGDSAAKSLLQGTIQMIQERLGSELIQGLGYRQAWALVGVIDGGSTSCNESVRNYEN
HSSGGKALAQREFYTVDGQKFSVTAYSEWIEGVSLSGFRVEVVDGVKLNLLDDVSS
WKPGDQIVVASTDYSMYQAEEFTLLPCSECSHFQVKVKETPQFLHMGEIIDGVDMRA
EVGILTRNIVIQGEVEDSCYAENQCQFFDYDTFGGHIMIMKNFTSVHLSYVELKHMG
QQQMGRYPVHFHLCGDVDYKGGYRHATFVDGLSIHHSFSRCITVHGTNGLLIKDTIG
FDTLGHCFFLEDGIEQRNTLFHNLGLLTKPGTLLPTDRNNSMCTTMRDKVFGNYIPVP
ATDCMAVSTFWIAHPNNNLINNAAAGSQDAGIWYLFHKEPTGESSGLQLLAKPELTP
LGIFYNNRVHSNFKAGLFIDKGVKTTNSSAADPREYLCLDNSARFRPHQDANPEKPR
VAALIDRLIAFKNNDNGAWVRGGDIIVQNSAFADNGIGLTFASDGSFPSDEGSSQEVS
ESLFVGESRNYGFQGGQNKYVGTGGIDQKPRTLPRNRTFPIRGFQIYDGPIHLTRSTFK
KYVPTPDRYSSAIGFLMKNSWQITPRNNISLVKFGPHVSLNVFFGKPGPWFEDCEMD
GDKNSIFHDIDGSVTGYKDAYVGRMDNYLIRHPSCVNVSKWNAVICSGTYAQVYVQ
TWSTQNLSMTITRDEYPSNPMVLRGINQKAAFPQYQPVVMLEKGYTIHWNGP APRTT
FLYLVNFNKNDWIRVGLCYPSNTSFQVTFGYLQRQNGSLSKIEEYEPVHSLEELQRKQ
SERKFYFDSSTGLLFLYLKAKSHRHGHSYCSSQGCERVKIQAATDSKDISNCMAKAY
PQYYRKPSVVKRMPAMLTGLCQGCGTRQVVFTSDPHKSYLPVQFQSPDKAETQRGD
PSVISVNGTDFTFRSAGVLLLVVDPCSVPFRLTEKTVFPLADVSRIEEYLKTGIPPRSIV
LLSTRGEIKQLNISHLLVPLGLAKPAHLYDKGSTIFLGFSGNFKPSWTKLFTSPAGQGL
GVLEQFIPLQLDEYGCPRATTVRRRDLELLKQASKAHGSSENLYFQGSSHHHHHH
(SEQ ID NO. 148)
DAAQPARRARRTKLGTSSKYAPDENCPDQNPRLRNWDPGQDSAKQVVIKEG
DMLRLTSDATVHSIVIQDGGLLVFGDNKDGSRNITLRTHYILIQDGGALHIGAEKCRY
KSKATITLYGKSDEGESMPTFGKKFIGVEAGGTLELHGARKASWTLLARTLNSSGLPF
GSYTFEKDFSRGLNVRVIDQDTAKILESERFDTHEYRNESRRLQEFLRFQDPGRIVAIA
VGDSAAKSLLQGTIQMIQERLGSELIQGLGYRQAWALVGVIDGGSTSCNESVRNYEN
HSSGGKALAQREFYTVDGQKFSVTAYSEWIEGVSLSGFRVEVVDGVKLNLLDDVSS
WKPGDQIVVASTDYSMYQAEEFTLLPCSECSHFQVKVKETPQFLHMGEIIDGVDMRA
EVGILTRNIVIQGEVEDSCYAENQCQFFDYDTFGGHIMIMKNFTSVHLSYVELKHMG
QQQMGRYPVHFHLCGDVDYKGGYRHATFVDGLSIHHSFSRCITVHGTNGLLIKDTIG
FDTLGHCFFLEDGIEQRNTLFHNLGLLTKPGTLLPTDRNNSMCTTMRDKVFGNYIPVP
ATDCMAVSTFWIAHPNNNLINNAAAGSQDAGIWYLFHKEPTGESSGLQLLAKPELTP
LGIFYNNRVHSNFKAGLFIDKGVKTTNSSAADPREYLCLDNSARFRPHQDANPEKPR
VAALIDRLIAFKNNDNGAWVRGGDIIVQNSAFADNGIGLTFASDGSFPSDEGSSQEVS
ESLFVGESRNYGFQGGQNKYVGTGGIDQKPRTLPRNRTFPIRGFQIYDGPIHLTRSTFK
KYVPTPDRYSSAIGFLMKNSWQITPRNNISLVKFGPHVSLNVFFGKPGPWFEDCEMD
GDKNSIFHDIDGSVTGYKDAYVGRMDNYLIRHPSCVNVSKWNAVICSGTYAQVYVQ
TWSTQNLSMTITRDEYPSNPMVLRGINQKAAFPQYQPVVMLEKGYTIHWNGP APRTT
FLYLVNFNKNDWIRVGLCYPSNTSFQVTFGYLQRQNGSLSKIEEYEPVHSLEELQRKQ
SERKFYFDSSTGLLFLYLKAKSHRHGHSYCSSQGCERVKIQAATDSKDISNCMAKAY
PQYYRKPSVVKRMPAMLTGLCQGCGTRQVVFTSDPHKSYLPVQFQSPDKAETQRGD
PSVISVNGTDFTFRSAGVLLLVVDPCSVPFRLTEKTVFPLADVSRIEEYLKTGIPPRSIV
LLSTRGEIKQLNISHLLVPLGLAKPAHLYDKGSTIFLGFSGNFKPSWTKLFTSPAGQGL
GVLEQFIPLQLDEYGCPRATTVRRRDLELLKQASKAHGSSENLYFQGSS
(SEQ ID NO. 150)
DAAQPARRARRTKLGTSSKYAPDENCPDQNPRLRNWDPGQDSAKQVVIKEG
DMLRLTSDATVHSIVIQDGGLLVFGDNKDGSRNITLRTHYILIQDGGALHIGAEKCRY
KSKATITLYGKSDEGESMPTFGKKFIGVEAGGTLELHGARKASWTLLARTLNSSGLPF
GSYTFEKDFSRGLNVRVIDQDTAKILESERFDTHEYRNESRRLQEFLRFQDPGRIVAIA
VGDSAAKSLLQGTIQMIQERLGSELIQGLGYRQAWALVGVIDGGSTSCNESVRNYEN
HSSGGKALAQREFYTVDGQKFSVTAYSEWIEGVSLSGFRVEVVDGVKLNLLDDVSS
WKPGDQIVVASTDYSMYQAEEFTLLPCSECSHFQVKVKETPQFLHMGEIIDGVDMRA
EVGILTRNIVIQGEVEDSCYAENQCQFFDYDTFGGHIMIMKNFTSVHLSYVELKHMG
QQQMGRYPVHFHLCGDVDYKGGYRHATFVDGLSIHHSFSRCITVHGTNGLLIKDTIG
FDTLGHCFFLEDGIEQRNTLFHNLGLLTKPGTLLPTDRNNSMCTTMRDKVFGNYIPVP
ATDCMAVSTFWIAHPNNNLINNAAAGSQDAGIWYLFHKEPTGESSGLQLLAKPELTP
LGIFYNNRVHSNFKAGLFIDKGVKTTNSSAADPREYLCLDNSARFRPHQDANPEKPR
VAALIDRLIAFKNNDNGAWVRGGDIIVQNSAFADNGIGLTFASDGSFPSDEGSSQEVS
ESLFVGESRNYGFQGGQNKYVGTGGIDQKPRTLPRNRTFPIRGFQIYDGPIHLTRSTFK
KYVPTPDRYSSAIGFLMKNSWQITPRNNISLVKFGPHVSLNVFFGKPGPWFEDCEMD
GDKNSIFHDIDGSVTGYKDAYVGRMDNYLIRHPSCVNVSKWNAVICSGTYAQVYVQ
TWSTQNLSMTITRDEYPSNPMVLRGINQKAAFPQYQPVVMLEKGYTIHWNGPAPRTT
FLYLVNFNKNDWIRVGLCYPSNTSFQVTFGYLQRQNGSLSKIEEYEPVHSLEELQRKQ
SERKFYFDSSTGLLFLYLKAKSHRHGHSYCSSQGCERVKIQAATDSKDISNCMAKAY
PQYYRKPSVVKRMPAMLTGLCQGCGTRQVVFTSDPHKSYLPVQFQSPDKAETQRGD
PSVISVNGTDFTFRSAGVLLLVVDPCSVPFRLTEKTVFPLADVSRIEEYLKTGIPPRSIV
LLSTRGEIKQLNISHLLVPLGLAKPAHLYDKGSTIFLGFSGNFKPSWTKLFTSPAGQGL
GVLEQFIPLQLDEYGCPRATTVRRRDLELLKQASKAH
(SEQ ID NO. 152)
GTSSKYAPDENCPDQNPRLRNWDPGQDSAKQVVIKEGDMLRLTSDATVHSIV
IQDGGLLVFGDNKDGSRNITLRTHYILIQDGGALHIGAEKCRYKSKATITLYGKSDEG
ESMPTFGKKFIGVEAGGTLELHGARKASWTLLARTLNSSGLPFGSYTFEKDFSRGLNV
RVIDQDTAKILESERFDTHEYRNESRRLQEFLRFQDPGRIVAIAVGDSAAKSLLQGTIQ
MIQERLGSELIQGLGYRQAWALVGVIDGGSTSCNESVRNYENHSSGGKALAQREFYT
VDGQKFSVTAYSEWIEGVSLSGFRVEVVDGVKLNLLDDVSSWKPGDQIVVASTDYS
MYQAEEFTLLPCSECSHFQVKVKETPQFLHMGEIIDGVDMRAEVGILTRNIVIQGEVE
DSCYAENQCQFFDYDTFGGHIMIMKNFTSVHLSYVELKHMGQQQMGRYPVHFHLC
GDVDYKGGYRHATFVDGLSIHHSFSRCITVHGTNGLLIKDTIGFDTLGHCFFLEDGIEQ
RNTLFHNLGLLTKPGTLLPTDRNNSMCTTMRDKVFGNYIPVPATDCMAVSTFWIAHP
NNNLINNAAAGSQDAGIWYLFHKEPTGESSGLQLLAKPELTPLGIFYNNRVHSNFKA
GLFIDKGVKTTNSSAADPREYLCLDNSARFRPHQDANPEKPRVAALIDRLIAFKNNDN
GAWVRGGDIIVQNSAFADNGIGLTFASDGSFPSDEGSSQEVSESLFVGESRNYGFQGG
QNKYVGTGGIDQKPRTLPRNRTFPIRGFQIYDGPIHLTRSTFKKYVPTPDRYSSAIGFL
MKNSWQITPRNNISLVKFGPHVSLNVFFGKPGPWFEDCEMDGDKNSIFHDIDGSVTG
YKDAYVGRMDNYLIRHPSCVNVSKWNAVICSGTYAQVYVQTWSTQNLSMTITRDE
YPSNPMVLRGINQKAAFPQYQPVVMLEKGYTIHWNGPAPRTTFLYLVNFNKNDWIR
VGLCYPSNTSFQVTFGYLQRQNGSLSKIEEYEPVHSLEELQRKQSERKFYFDSSTGLLF
LYLKAKSHRHGHSYCSSQGCERVKIQAATDSKDISNCMAKAYPQYYRKPSVVKRMP
AMLTGLCQGCGTRQVVFTSDPHKSYLPVQFQSPDKAETQRGDPSVISVNGTDFTFRS
AGVLLLVVDPCSVPFRLTEKTVFPLADVSRIEEYLKTGIPPRSIVLLSTRGEIKQLNISH
LLVPLGLAKPAHLYDKGSTIFLGFSGNFKPSWTKLFTSPAGQGLGVLEQFIPLQLDEY
GCPRATTVRRRDLELLKQASKAHGSSENLYFQGSS

### Protein

Some embodiments provide a recombinant TMEM2 protein or fragment thereof, comprising at least 1000 amino acid residues. In some embodiments, the recombinant TMEM2 protein or fragment thereof includes a catalytic domain and can additionally include other domains. In some embodiments, the recombinant TMEM2 protein or fragment thereof comprises least 1000 amino acid residues of TMEM2 extracellular domain.

In some embodiments, the recombinant TMEM2 protein or fragment thereof comprises 1000 to 1280 amino acid residues of TMEM2 extracellular domain. In some embodiments, the recombinant TMEM2 protein or fragment thereof is human TMEM2 extracellular domain.

In some embodiments, the recombinant TMEM2 protein or fragment thereof further comprises at least one N-linked sugar moiety. In some embodiments, the recombinant TMEM2 protein or fragment thereof further comprises at least two, three, four, five, six seven, eight, nine, ten, eleven, twelve, thirteen or fourteen N-linked sugar moieties, or number of N-linked sugar moieties within a range defined by any of the preceding values. There are fourteen potential N-linked glycosylation sites at residues 171, 248, 292, 362, 525, 636, 739, 914, 980, 1005, 1078, 1092, 1234, or 1299 of the human TMEM2 protein exemplified in SEQ ID NO: 3. In some embodiments, the recombinant TMEM2 protein or fragment thereof further comprises at least one N-linked sugar moiety at positions selected from residues 68, 145, 189, 259, 422, 533, 636, 811, 877, 902, 975, 989, 1131, or 1195 of the recombinant TMEM2 protein or fragment thereof exemplified in SEQ ID NO. 10, SEQ ID NO. 148, SEQ ID NO. 150, or SEQ ID NO. 152. In some embodiments, the recombinant TMEM2 protein or fragment thereof comprises an amino acid sequence as set forth in SEQ ID NO. 8, e.g., at the N-terminus of the recombinant TMEM2 protein or fragment. In some embodiments, the recombinant TMEM2 protein or fragment thereof comprises an amino acid sequence as set forth in SEQ ID NO. 5, e.g., at the C-terminus of the recombinant TMEM2 protein or fragment. In some embodiments, the recombinant TMEM2 protein or fragment thereof comprises an amino acid sequence as set forth in SEQ ID NO: 6, e.g., at the C-terminus of the recombinant TMEM2 protein or fragment. In some embodiments, the recombinant TMEM2 protein or fragment thereof comprises an amino acid sequence as set forth in SEQ ID NO: 9, e.g., at the C-terminus of the recombinant TMEM2 protein or fragment.

Some embodiments provide a recombinant protein comprising at least 1000 amino acid residues. In some embodiments, the recombinant protein includes a catalytic domain and can additionally include other domains. In some embodiments, the recombinant protein comprises least 1000 amino acid residues of TMEM2 extracellular domain.

In some embodiments, the recombinant TMEM2 protein 1000 to 1280 amino acid residues of TMEM2 extracellular domain. In some embodiments, the recombinant protein is human TMEM2 extracellular domain.

In some embodiments, the recombinant protein further comprises at least one N-linked sugar moiety. In some embodiments, the recombinant TMEM2 protein further comprises at least two, three, four, five, six seven, eight, nine, ten, eleven, twelve, thirteen or fourteen N-linked sugar moieties, or number of N-linked sugar moieties within a range defined by any of the preceding values. There are fourteen potential N-linked glycosylation sites at residues 171, 248, 292, 362, 525, 636, 739, 914, 980, 1005, 1078, 1092, 1234, or 1299 of the human TMEM2 protein exemplified in SEQ ID NO. 3. In some embodiments, the recombinant protein further comprises at least one N-linked sugar moiety at positions selected from residues 68, 145, 189, 259, 422, 533, 636, 811, 877, 902, 975, 989, 1131, or 1195 of the recombinant protein exemplified in SEQ ID NO. 10, SEQ ID NO. 148, SEQ ID NO. 150, or SEQ ID NO. 152. In some embodiments, the recombinant protein comprises an amino acid sequence as set forth in SEQ ID NO. 8, e.g., at the N-terminus of the recombinant protein. In some embodiments, the recombinant protein comprises an amino acid sequence as set forth in SEQ ID NO. 5, e.g., at the C-terminus of the recombinant protein. In some embodiments, the recombinant protein comprises an amino acid sequence as set forth in SEQ ID NO. 6, e.g., at the C-terminus of the recombinant protein. In some embodiments, the recombinant protein comprises an amino acid sequence as set forth in SEQ ID NO. 9 (HHHHHH), e.g., at the C-terminus of the recombinant protein. In some embodiments, the recombinant protein has at least 93% amino acid sequence identity to the amino acid sequence as set forth in to SEQ ID NO. 3. In some embodiments, the recombinant protein comprises a mutation. In some embodiments, the mutation is selected from a deletion, insertion, or substitution. In some embodiments, the mutation alters the enzymatic activity of the recombinant protein. In some embodiments, the mutation abrogates the enzyme activity of the recombinant protein. In some embodiments, the mutation completely abrogates the mutation of the recombinant protein.

TABLE 3
Exemplary N-terminus and C-terminus sequences of recombinant TMEM2
protein or fragment.

| Description | Sequence | SEQ ID NO: |
|---|---|---|
| C-terminus of recombinant hTMEM2 | GSSENLYFQGSSHHHHHH | 5 |
| C-terminus of recombinant hTMEM2 | GSSENLYFQGSS | 6 |
| (without H-tag)[ | | |
| Vector-derived sequence remaining | DAAQPARRARRTKLGT | 8 |
| at the N-terminus of recombinant | | |
| hTMEM2 | | |

In some embodiments, the recombinant TMEM2 protein or fragment thereof further comprises at least one linked polymer. In some embodiments, the recombinant TMEM2 protein or fragment thereof further comprises at least two, three, four, five, six or seven N-linked polymers. Some embodiments provide a recombinant protein having at least at least 93% identity to the amino acid sequence as set forth in to SEQ ID NO. 1. In some embodiments, the recombinant protein further comprises at least one linked polymer. In some embodiments, the recombinant protein further comprises at least two, three, four, five, six or seven N-linked polymers. In some embodiments, the recombinant protein has less than 100% or less than 98% amino acid sequence identity to the amino acid sequence as set forth in to SEQ ID NO. 1. In some embodiments, the recombinant protein comprises, consists essentially of, or consists of 1280, 1288, 1290, 1296, 1298, 1302 or 1314 amino acid residues, or number of amino acid residues within a range defined by any of the preceding values. In some embodiments, the recombinant protein comprises an amino acid sequence having 100% identity to the amino acid sequence set forth in SEQ ID NO. 1.

In some embodiments, the recombinant TMEM2 protein or fragment thereof further comprises at least one linked polymer. In some embodiments, the recombinant TMEM2 protein or fragment thereof further comprises at least two, three, four, five, six or seven N-linked polymers, or number of polymers within a range defined by any of the preceding values.

In some embodiments, the polymer is PEG or commercially available modified PEG.

Aspects of the present specification disclose, in part, PEGylation of the recombinant TMEM2 protein or fragment thereof. PEGylation involves the formation of a stable covalent attachment of one or more synthetic poly(ethylene glycol) (PEG) polymers to a compound of interest. PEG polymers are typically biologically inert, non-immunogenic molecules that confer greater water solubility to compounds by forming a hydrophilic shell. PEG polymers are synthesized by the polymerization of ethylene oxide and are commercially available over a wide range of molecular weights from 100 g/mol to 10,000,000 g/mol. PEG polymers can have a linear or branched structure. Branched PEG polymers can increase the size of the total conjugate without resultant increase in number of attachment sites and have been shown to improve stability in response to changes in pH, proteolytic digestion, and temperature change as compared to linear PEG polymers. Depending on how one chooses to define the constituent monomer (as ethylene glycol, ethylene oxide or oxyethylene), PEG polymers are also known as polyethylene oxide (PEO) polymers and polyoxyethylene (POE) polymers. In some embodiments, the PEG polymer includes a terminal OCH₃ instead of a terminal OH. In some embodiments, the PEG polymer includes a terminal OCH₂CH₃ instead of a terminal OH.

The presence of one or more PEG polymers can alter various physicochemical properties of a compound including, without limitation, increasing the size and molecular weight of the compound.

Formation of a PEG-conjugated compound involves the activation of PEG polymers by preparing derivatives with functional groups at one or both of the terminal ends of the polymers. PEG polymer derivatives with functional groups at both terminal ends can be homobifunctional (identical reactive groups at either end) and heterobifunctional (different reactive groups at either end). PEG polymers can be an oxidized, reduced, aminated and/or hydrazide derivative. Useful functional groups include, without limitation, amine reactive PEG polymers activate by, e.g., the presence of an N-hydroxy-succinimide (NHS) ester that react with amine groups; and sulfhydryl-reactive PEG polymers activate by, e.g., the presence of a maleimide group that react sulfhydryl groups. Covalent attachment of an activated PEG polymer is generally made at the ∞ or ε amino groups of lysine, a β-aspartic acid, a γ-glutamic acid, or any other amino acid have a free amino group; the N-terminal amino group of cysteine, histidine, arginine, aspartic acid, glutamic acid, serine, threonine, tyrosine; or the C-terminal carboxylic acid. Among these amino acids, a choice for PEGylation is lysine and N-terminal amino group.

The exact molecular weight of a PEG polymer used to conjugate to a compound can vary. In aspects of this embodiment, the number of monomers comprising an activated PEG polymers useful to modify a compound disclosed herein can be, e.g., about 4 monomers, about 5 monomers, about 6 monomers, about 7 monomers, about 8 monomers, about 9 monomers, about 10 monomers, about 11 monomers, about 12 monomers, about 13 monomers, about 14 monomers, about 15 monomers, about 16 monomers, about 17 monomers, about 18 monomers, about 19 monomers, about 20 monomers, about 21 monomers, about 22 monomers, about 23 monomers, about 24 monomers, about 25 monomers, about 26 monomers, about 27 monomers, about 28 monomers, about 29 monomers or about 30 monomers. In other aspects of this embodiment, the number of monomers comprising an activated PEG polymers useful to modify a compound disclosed herein can be, e.g., at least 4 monomers, at least 5 monomers, at least 6 monomers, at least 7 monomers, at least 8 monomers, at least 9 monomers, at least 10 monomers, at least 11 monomers, at least 12 monomers, at least 13 monomers, at least 14 monomers, at least 15 monomers, at least 16 monomers, at least 17 monomers, at least 18 monomers, at least 19 monomers, at least 20 monomers, at least 21 monomers, at least 22 monomers, at least 23 monomers, at least 24 monomers, at least at least 25 monomers, at least 26 monomers, at least 27 monomers, at least 28 monomers, at least 29 monomers or at least 30 monomers. In yet other aspects of this embodiment, the number of monomers comprising an activated PEG polymers useful to modify a compound disclosed herein can be, e.g., at most 4 monomers, at most 5 monomers, at most 6 monomers, at most 7 monomers, at most 8 monomers, at most 9 monomers, at most 10 monomers, at most 11 monomers, at most 12 monomers, at most 13 monomers, at most 14 monomers, at most 15 monomers, at most 16 monomers, at most 17 monomers, at most 18 monomers, at most 19 monomers, at most 20 monomers, at most 21 monomers, at most 22 monomers, at most 23 monomers, at most 24 monomers, at most at least 25 monomers, at most 26 monomers, at most 27 monomers, at most 28 monomers, at most 29 monomers or at most 30 monomers. In some embodiments, the PEG polymer includes a terminal OCH₃ instead of a terminal OH. In some embodiments, the PEG polymer includes a terminal OCH₂CH₃ instead of a terminal OH.

In some embodiments, the number of monomers comprising an activated PEG polymers useful to modify a compound disclosed herein can be, e.g., about 4 to about 8 monomers, about 4 to about 12 monomers, about 4 to about 16 monomers, about 4 to about 20 monomers, about 4 to about 24 monomers, about 4 to about 28 monomers, about 4 to about 32 monomers, about 6 to about 8 monomers, about 6 to about 12 monomers, about 6 to about 16 monomers, about 6 to about 20 monomers, about 6 to about 24 monomers, about 6 to about 28 monomers, about 6 to about 32 monomers, about 8 to about 12 monomers, about 8 to about 16 monomers, about 8 to about 20 monomers, about 8 to about 24 monomers, about 8 to about 28 monomers, about 8 to about 32 monomers, about 10 to about 12 monomers, about 10 to about 16 monomers, about 10 to about 20 monomers, about 10 to about 24 monomers, about 10 to about 28 monomers, about 10 to about 32 monomers, about 12 to about 16 monomers, about 12 to about 20 monomers, about 12 to about 24 monomers, about 12 to about 28 monomers, about 12 to about 32 monomers, about 14 to about 16 monomers, about 14 to about 20 monomers, about 14 to about 24 monomers, about 14 to about 28 monomers, about 14 to about 32 monomers, about 16 to about 20 monomers, about 16 to about 24 monomers, about 16 to about 28 monomers, about 16 to about 32 monomers, about 18 to about 20 monomers, about 18 to about 24 monomers, about 18 to about 28 monomers, about 18 to about 32 monomers, about 20 to about 24 monomers, about 20 to about 28 monomers, about 20 to about 32 monomers, about 22 to about 24 monomers, about 22 to about 28 monomers, about 22 to about 32 monomers, about 24 to about 28 monomers, about 24 to about 32 monomers, about 26 to about 28 monomers, about 26 to about 32 monomers, about 28 to about 32 monomers or about 30 to about 32 monomers. In some embodiments, the PEG polymer includes a terminal OCH₃ instead of a terminal OH. In some embodiments, the PEG polymer includes a terminal OCH₂CH₃ instead of a terminal OH.

In some embodiments, the molecular weight of the PEG polymer can be, e.g., about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, or about 100 kDa. In other aspects of this embodiment, the molecular weight of the PEG polymer derivative can be, e.g., at least 20 kDa, at least 25 kDa, at least 30 kDa, at least 35 kDa, at least 40 kDa, at least 45 kDa, at least 50 kDa, at least 55 kDa, at least 60 kDa, at least 65 kDa, at least 70 kDa, at least 75 kDa, at least 80 kDa, at least 85 kDa, at least 90 kDa, at least 95 kDa, or at least 100 kDa. In yet other aspects of this embodiment, the molecular weight of the activated PEG polymer derivative can be, e.g., at most 20 kDa, at most 25 kDa, at most 30 kDa, at most 35 kDa, at most 40 kDa, at most 45 kDa, at most 50 kDa, at most 55 kDa, at most 60 kDa, at most 65 kDa, at most 70 kDa, at most 75 kDa, at most 80 kDa, at most 85 kDa, at most 90 kDa, at most 95 kDa, or at most 100 kDa. In still other aspects of this embodiment, the molecular weight of the activated PEG polymer derivative can be, e.g., about 20 kDa to about 30 kDa, about 20 kDa to about 40 kDa, about 20 kDa to about 50 kDa, about 20 kDa to about 60 kDa, about 20 kDa to about 70 kDa, about 20 kDa to about 80 kDa, about 20 kDa to about 90 kDa, about 20 kDa to about 100 kDa, about 30 kDa to about 40 kDa, about 30 kDa to about 50 kDa, about 30 kDa to about 60 kDa, about 30 kDa to about 70 kDa, about 30 kDa to about 80 kDa, about 30 kDa to about 90 kDa, about 30 kDa to about 100 kDa, about 40 kDa to about 50 kDa, about 40 kDa to about 60 kDa, about 40 kDa to about 70 kDa, about 40 kDa to about 80 kDa, about 40 kDa to about 90 kDa, about 40 kDa to about 100 kDa, about 50 kDa to about 60 kDa, about 50 kDa to about 70 kDa, about 50 kDa to about 80 kDa, about 50 kDa to about 90 kDa, about 50 kDa to about 100 kDa, about 60 kDa to about 70 kDa, about 60 kDa to about 80 kDa, about 60 kDa to about 90 kDa, about 60 kDa to about 100 kDa, about 70 kDa to about 80 kDa, about 70 kDa to about 90 kDa, about 70 kDa to about 100 kDa, about 80 kDa to about 90 kDa, about 80 kDa to about 100 kDa, or about 90 kDa to about 100 kDa. In some embodiments, the PEG polymer includes a terminal OCH₃ instead of a terminal OH. In some embodiments, the PEG polymer includes a terminal OCH₂CH₃ instead of a terminal OH.

In some embodiments, the molecular weight of the PEG-conjugated compound can be, e.g., about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, or about 100 kDa. In other aspects of this embodiment, the molecular weight of the PEG-conjugated compound can be, e.g., at least 20 kDa, at least 25 kDa, at least 30 kDa, at least 35 kDa, at least 40 kDa, at least 45 kDa, at least 50 kDa, at least 55 kDa, at least 60 kDa, at least 65 kDa, at least 70 kDa, at least 75 kDa, at least 80 kDa, at least 85 kDa, at least 90 kDa, at least 95 kDa, or at least 100 kDa. In yet other aspects of this embodiment, the molecular weight of the PEG-conjugated compound can be, e.g., at most 20 kDa, at most 25 kDa, at most 30 kDa, at most 35 kDa, at most 40 kDa, at most 45 kDa, at most 50 kDa, at most 55 kDa, at most 60 kDa, at most 65 kDa, at most 70 kDa, at most 75 kDa, at most 80 kDa, at most 85 kDa, at most 90 kDa, at most 95 kDa, or at most 100 kDa. In still other aspects of this embodiment, the molecular weight of the PEG-conjugated compound can be, e.g., about 20 kDa to about 30 kDa, about 20 kDa to about 40 kDa, about 20 kDa to about 50 kDa, about 20 kDa to about 60 kDa, about 20 kDa to about 70 kDa, about 20 kDa to about 80 kDa, about 20 kDa to about 90 kDa, about 20 kDa to about 100 kDa, about 30 kDa to about 40 kDa, about 30 kDa to about 50 kDa, about 30 kDa to about 60 kDa, about 30 kDa to about 70 kDa, about 30 kDa to about 80 kDa, about 30 kDa to about 90 kDa, about 30 kDa to about 100 kDa, about 40 kDa to about 50 kDa, about 40 kDa to about 60 kDa, about 40 kDa to about 70 kDa, about 40 kDa to about 80 kDa, about 40 kDa to about 90 kDa, about 40 kDa to about 100 kDa, about 50 kDa to about 60 kDa, about 50 kDa to about 70 kDa, about 50 kDa to about 80 kDa, about 50 kDa to about 90 kDa, about 50 kDa to about 100 kDa, about 60 kDa to about 70 kDa, about 60 kDa to about 80 kDa, about 60 kDa to about 90 kDa, about 60 kDa to about 100 kDa, about 70 kDa to about 80 kDa, about 70 kDa to about 90 kDa, about 70 kDa to about 100 kDa, about 80 kDa to about 90 kDa, about 80 kDa to about 100 kDa, or about 90 kDa to about 100 kDa. In some embodiments, the PEG polymer includes a terminal OCH₃ instead of a terminal OH. In some embodiments, the PEG polymer includes a terminal OCH₂CH₃ instead of a terminal OH.

In some embodiments, the recombinant TMEM2 protein or fragment thereof comprises, consists essentially of, or consists of a complete sequence of amino acids set forth in SEQ ID NO. 3. In some embodiments, the recombinant protein comprises, consists essentially of, or consists of a complete sequence of amino acids set forth in SEQ ID NO. 3. In some embodiments, the recombinant protein comprises, consists essentially of, or consists of 1280, 1288, 1290, 1296, 1298, 1302 or 1314 amino acid residues, or number of amino acid residues within a range defined by any of the preceding values.

In some embodiments, the recombinant TMEM2 protein or fragment thereof comprises does not comprise the complete sequence of amino acids set forth in SEQ ID NO. 1, or the recombinant TMEM2 protein or fragment thereof has at least 95% amino acid sequence identity with a sequence of amino acids set forth in SEQ ID NO. 3.

In some embodiments, the recombinant TMEM2 protein or fragment thereof does not comprise, consist essentially of, or consist of the complete sequence of amino acids set forth in SEQ ID NO. 1, or the recombinant TMEM2 protein or fragment thereof has at least 93% amino acid sequence identity with a sequence of amino acids set forth in SEQ ID NO. 3. In some embodiments, the recombinant protein comprises, consists essentially of, or consists of 1280, 1288, 1290, 1296, 1298, 1302 or 1314 amino acid residues, or number of amino acid residues within a range defined by any of the preceding values.

In some embodiments, the recombinant TMEM2 protein or fragment thereof consists of a contiguous sequence of amino acid residues contained within SEQ ID NO. 1 that includes at least amino acids 104-1383 of SEQ ID NO. 3. In some embodiments, the recombinant TMEM2 protein or fragment thereof consists of a contiguous sequence of amino acid residues contained within SEQ ID NO. 10, 148, 150, or 152 that includes at least amino acids 104-1383 of SEQ ID NO. 3. In some embodiments, the recombinant TMEM2 protein or fragment thereof has at least 95%, 96% or 97% amino acid sequence identity with a sequence of amino acids set forth in SEQ ID NO. 1. In some embodiments, the recombinant TMEM2 protein or fragment thereof has less than 100% or less than 98% amino acid sequence identity with a sequence of amino acids set forth in SEQ ID NO. 1.

In some embodiments, the recombinant TMEM2 protein or fragment thereof has at least 80%, 85%, 90% or 93% amino acid sequence identity with a sequence of amino acids set forth in SEQ ID NO. 1. In some embodiments, the recombinant TMEM2 protein or fragment thereof has less than 100%, less than 95%, or less than 94% amino acid sequence identity with a sequence of amino acids set forth in SEQ ID NO. 1.

In some embodiments, (i) the recombinant TMEM2 protein or fragment thereof comprises a sequence of amino acids set forth as amino acids 104-113, 114-123, 124-133, 134-143, 144-153, 154-163, 164-173, 174-183, 184-193, 194-203, 204-213, 214-223, 224-233, 234-243, 244-253, 254-263, 264-273, 274-283, 284-293, 294-303-, 304-313, 314-323, 324-333, 334-343, 344-353, 354-363, 364-373, 374-383, 384-393, 394-403, 404-413, 414-423, 424-433, 434-443, 444-453, 454-463, 464-473, 474-483, 484-493, 494-503, 504-513, 514-523, 524-533, 534-543, 544-553, 554-563, 564-573, 574-583, 584-593, 594-603, 604-613, 614-623, 624-633, 634-643, 644-653, 654-663, 664-673, 674-683, 684-693, 694-703, 704-713, 714-723, 724-733, 734-743, 744-753, 7554-763, 764-773, 774-783, 784-793, 794-803, 804-813, 814-823, 824-833,834-843,844-853,854-863,864-873,874-883,884-893,894-903,904-913,914-923,924-933, 934-943, 944-953, 954-963, 964-973, 974-983, 984-993, 994-1003, 1004-1013, 1014-1023, 1024-1033, 1034-1043, 1044-1053, 1054-1063, 1064-1073, 1074-1083, 1084-1093, 1094-1103, 1104-1113, 1114-1123, 1124-1133, 1134-1143, 1144-1153, 1154-1163, 1164-1173, 1174-1183, 1184-1193, 1194-1203, 1204-1213, 1214-1213, 1214-1223, 1224-1233, 1234-1243, 1244-1253, 1254-1263, 1264-1273, 1274-1283, 1284-1293, 1294-1303, 1304-1313, 1314-1323, 1324-1333, 1334-1343, 1344-1353, 1354-1363, 1364-1373, or 1374-1383 of SEQ ID NO. 3; or (ii) the recombinant protein contains amino acid substitutions in the sequence of amino acids set forth as amino acids 104-113, 114-123, 124-133, 134-143, 144-153, 154-163, 164-173, 174-183, 184-193, 194-203, 204-213, 214-223, 224-233, 234-243, 244-253, 254-263, 264-273, 274-283, 284-293, 294-303-, 304-313, 314-323, 324-333, 334-343, 344-353, 354-363, 364-373, 374-383, 384-393, 394-403, 404-413, 414-423, 424-433, 434-443, 444-453, 454-463, 464-473, 474-483, 484-493, 494-503, 504-513, 514-523, 524-533, 534-543, 544-553, 554-563,564-573,574-583,584-593,594-603,604-613,614-623,624-633,634-643,644-653, 654-663, 664-673, 674-683, 684-693, 694-703, 704-713, 714-723, 724-733, 734-743, 744-753, 7554-763, 764-773, 774-783, 784-793, 794-803, 804-813, 814-823, 824-833, 834-843, 844-853,854-863,864-873,874-883,884-893,894-903,904-913,914-923,924-933,934-943,944-953, 954-963, 964-973, 974-983, 984-993, 994-1003, 1004-1013, 1014-1023, 1024-1033, 1034-1043, 1044-1053, 1054-1063, 1064-1073, 1074-1083, 1084-1093, 1094-1103, 1104-1113, 1114-1123, 1124-1133, 1134-1143, 1144-1153, 1154-1163, 1164-1173, 1174-1183, 1184-1193, 1194-1203, 1204-1213, 1214-1213, 1214-1223, 1224-1233, 1234-1243, 1244-1253, 1254-1263, 1264-1273, 1274-1283, 1284-1293, 1294-1303, 1304-1313, 1314-1323, 1324-1333, 1334-1343, 1344-1353, 1354-1363, 1364-1373, or 1374-1383 of SEQ ID NO. 3, whereby the amino acid-substituted recombinant protein consists of a sequence of amino acids that has at least 95% amino acid sequence identity with the sequence of amino acids set forth as amino acids 104-113, 114-123, 124-133, 134-143, 144-153, 154-163, 164-173, 174-183, 184-193, 194-203, 204-213, 214-223, 224-233, 234-243, 244-253, 254-263, 264-273, 274-283, 284-293, 294-303-, 304-313, 314-323, 324-333, 334-343, 344-353, 354-363, 364-373, 374-383, 384-393, 394-403, 404-413, 414-423, 424-433, 434-443, 444-453, 454-463, 464-473, 474-483, 484-493, 494-503, 504-513, 514-523, 524-533, 534-543, 544-553, 554-563, 564-573, 574-583, 584-593, 594-603, 604-613, 614-623, 624-633, 634-643, 644-653, 654-663, 664-673, 674-683, 684-693, 694-703, 704-713, 714-723, 724-733, 734-743, 744-753, 7554-763, 764-773, 774-783, 784-793, 794-803, 804-813, 814-823, 824-833, 834-843, 844-853, 854-863, 864-873, 874-883, 884-893, 894-903, 904-913, 914-923, 924-933, 934-943, 944-953, 954-963, 964-973, 974-983, 984-993, 994-1003, 1004-1013, 1014-1023, 1024-1033, 1034-1043, 1044-1053, 1054-1063, 1064-1073, 1074-1083, 1084-1093, 1094-1103, 1104-1113, 1114-1123, 1124-1133, 1134-1143, 1144-1153, 1154-1163, 1164-1173, 1174-1183, 1184-1193, 1194-1203, 1204-1213, 1214-1213, 1214-1223, 1224-1233, 1234-1243, 1244-1253, 1254-1263, 1264-1273, 1274-1283, 1284-1293, 1294-1303, 1304-1313, 1314-1323, 1324-1333, 1334-1343, 1344-1353, 1354-1363, 1364-1373, or 1374-1383 of SEQ ID NO. 3.

In some embodiments, the recombinant TMEM2 protein or fragment thereof has at least 95%, 96%, 97%, 98%, or 99% amino acid sequence identity with a sequence of amino acids set forth in SEQ ID NO. 1. In some embodiments, the recombinant TMEM2 protein or fragment thereof over 99% amino acid sequence identity with a sequence of amino acids set forth in SEQ ID NO. 1. In some embodiments, (i) the recombinant TMEM2 protein or fragment thereof comprises a sequence of amino acids set forth as amino acids 1-10, 11-20, 21-30, 31-40, 41-50, 51-60, 61-70, 71-80, 81-90, 91-100, 101-110, 111-120, 121-130, 131-140, 141-150, 151-160, 161-170, 171-180, 181-190,191-200, 201-210, 211-220, 221-230, 231-240, 241-250, 251-260, 261-270, 271-280, 281-290, 291-300, 301-310, 311-320, 321-330, 331-340, 341-350, 351-360, 361-370, 371-380, 381-390, 391-400, 401-410, 411-420, 421-430, 431-440, 441-450, 451-460, 461-470, 471-480, 481-490, 491-500, 501-510, 511-520, 521-530, 531-540, 541-550, 551-560, 561-570, 571-580, 581-590, 591-600, 601-610, 611-620, 621-630, 631-640, 641-650, 651-660, 661-670, 671-680, 681-690, 691-700, 701-710, 711-720, 721-730, 731-740, 741-750, 751-760, 761-770, 771-780, 781-790, 791-800, 801-810, 811-820, 821-830, 831-840, 841-850, 851-860, 861-870, 871-880, 881-890, 891-900, 901-910, 911-920, 921-930, 931-940, 941-950, 951-960, 961-970, 971-980, 981-990, 991-1000, 1001-1010, 1011-1020, 1021-1030, 1031-1040, 1041-1050, 1051-1060, 1061-1070, 1071-1080, 1081-1090, 1091-1100, 1101-1110, 1111-1120, 1121-1130, 1131-1140, 1141-1150, 1151-1160, 1161-1170, 1171-1180, 1181-1190, 1191-1200, 1201-1210, 1211-1220, 1221-1230, 1231-1240, 1241-1250, 1251-1260, 1261-1270, or 1271-1280 of SEQ ID NO. 1; or (ii) the recombinant protein contains amino acid substitutions in the sequence of amino acids set forth as amino acids 1-10, 11-20, 21-30, 31-40, 41-50, 51-60, 61-70, 71-80, 81-90, 91-100, 101-110,111-120, 121-130, 131-140,141-150, 151-160,161-170,171-180,181-190,191-200,201-210,211-220,221-230,231-240,241-250,251-260,261-270,271-280,281-290,291-300,301-310, 311-320, 321-330, 331-340, 341-350, 351-360, 361-370, 371-380, 381-390, 391-400, 401-410, 411-420, 421-430, 431-440, 441-450, 451-460, 461-470, 471-480,481-490,491-500,501-510, 511-520, 521-530, 531-540, 541-550, 551-560, 561-570, 571-580, 581-590, 591-600, 601-610, 611-620, 621-630, 631-640, 641-650, 651-660, 661-670, 671-680, 681-690, 691-700, 701-710, 711-720, 721-730, 731-740, 741-750, 751-760, 761-770, 771-780,781-790,791-800,801-810, 811-820, 821-830,831-840, 841-850, 851-860,861-870,871-880, 881-890, 891-900, 901-910, 911-920, 921-930, 931-940, 941-950, 951-960, 961-970, 971-980, 981-990, 991-1000, 1001-1010, 1011-1020, 1021-1030, 1031-1040, 1041-1050, 1051-1060, 1061-1070, 1071-1080, 1081-1090, 1091-1100, 1101-1110, 1111-1120, 1121-1130, 1131-1140, 1141-1150, 1151-1160, 1161-1170, 1171-1180, 1181-1190, 1191-1200, 1201-1210, 1211-1220, 1221-1230, 1231-1240, 1241-1250, 1251-1260, 1261-1270, or 1271-1280 of SEQ ID NO. 1, whereby the amino acid-substituted recombinant protein consists of a sequence of amino acids that has at least 95% amino acid sequence identity with the sequence of amino acids set forth as amino acids 1-10, 11-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200,201-210,211-220,221-230,231-240,241-250,251-260,261-270,271-280, 281-290, 291-300, 301-310, 311-320, 321-330, 331-340, 341-350, 351-360, 361-370, 371-380, 381-390, 391-400, 401-410, 411-420, 421-430, 431-440, 441-450, 451-460, 461-470, 471-480, 481-490, 491-500, 501-510, 511-520, 521-530, 531-540, 541-550, 551-560, 561-570, 571-580, 581-590, 591-600, 601-610, 611-620, 621-630, 631-640, 641-650, 651-660, 661-670, 671-680, 681-690, 691-700, 701-710, 711-720, 721-730, 731-740, 741-750, 751-760, 761-770, 771-780, 781-790, 791-800, 801-810, 811-820, 821-830, 831-840, 841-850, 851-860, 861-870, 871-880, 881-890, 891-900, 901-910, 911-920, 921-930, 931-940, 941-950, 951-960, 961-970, 971-980, 981-990, 991-1000, 1001-1010, 1011-1020, 1021-1030, 1031-1040, 1041-1050, 1051-1060, 1061-1070, 1071-1080, 1081-1090, 1091-1100, 1101-1110, 1111-1120, 1121-1130, 1131-1140, 1141-1150, 1151-1160, 1161-1170, 1171-1180, 1181-1190, 1191-1200, 1201-1210, 1211-1220, 1221-1230, 1231-1240, 1241-1250, 1251-1260, 1261-1270, or 1271-1280 of SEQ ID NO. 1.

In some embodiments, the recombinant TMEM2 protein or fragment thereof comprises at least two, three, four, five, six, seven, eight, nine, ten, 11, 12, 13, 14, 15, 16, 17, 18, or 19 sequences of amino acids set in any of SEQ ID NOs. 1, 3, 10, 148, 150, or 152. In some embodiments, the recombinant TMEM2 protein or fragment thereof comprises less than 23, or 24 sequences of amino acids set in any of SEQ ID NOs. 1, 3, 10, 148, 150, or 152 whereby the recombinant protein consists of a sequence of amino acids that has at least 95%, 96%, 97% or 98% amino acid sequence identity with the sequence of amino acids set forth in SEQ ID NO. 1. In some embodiments, the recombinant TMEM2 protein or fragment thereof comprises at least two, three, four, five, six, seven, eight, nine, or ten, sequences of amino acids set in any of SEQ ID Nos. 20-147. In some embodiments, the recombinant TMEM2 protein or fragment thereof comprises less than 7, or 8 sequences of amino acids set in any of SEQ ID NOs. 20-147 whereby the recombinant protein consists of a sequence of amino acids that has at least 95%, 96%, 97% or 98% amino acid sequence identity with the sequence of amino acids set forth in SEQ ID NO. 1, 10, 148, 150, or 152.

As will be appreciated by those of skill in the art, the recombinant TMEM2 protein or fragment thereof can have different catalytic activity in comparison to wild type TMEM2 extracellular domain based of various factors such as sequence identity or level of glycosylation. In some embodiments, the recombinant TMEM2 protein or fragment thereof has catalytic activity of at least 50%, at least 60%, at least 70%, at least 80% or at least 90% of wild type TMEM2 extracellular domain.

In one aspect, the disclosure provides a protein mutein of TMEM2 extracellular domain or fragment thereof. In some embodiments, that protein is a mutein of a polypeptide of 1000 to 1280 amino acid residues of TMEM2 extracellular domain or fragment thereof, wherein: up to about 50% of the amino acids are replaced with another amino acid; and the resulting polypeptide is a single chain or two chain polypeptide that has catalytic activity of at least 10% of 1000 to 1280 amino acid residues of TMEM2 extracellular domain. In some embodiments, up to about 10% of the amino acids of the protein are replaced with another amino acid. In some embodiments, the resulting polypeptide is a single chain or two chain polypeptide and has catalytic activity of at least 50%, at least 60%, at least 70%, at least 80% or at least 90% of wild type TMEM2 extracellular domain.

Modifications and changes can be made in the polypeptides of wild type TMEM2 extracellular domain and still obtain a molecule having similar characteristics as wild type TMEM2 extracellular domain (e.g., a conservative amino acid substitution). For example, certain amino acids can be substituted for other amino acids in a sequence without appreciable loss of wild type TMEM2 extracellular domain hyaluronidase activity. Because it is the interactive capacity and nature of a polypeptide that defines that polypeptide's biological functional activity, certain amino acid sequence substitutions can be made in a polypeptide sequence and nevertheless obtain a polypeptide with like properties to wild type TMEM2 extracellular domain.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as HA. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more percent (%) sequence identity to wild type TMEM2 extracellular domain.

As used herein, the term “percent (%) sequence identity” is defined as the percentage of amino acids in a candidate sequence that are identical with the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given amino acids sequence C to, with, or against a given amino acids sequence D (which can alternatively be phrased as a given sequence C that has or comprises a certain % sequence identity to, with, or against a given sequence D) is calculated as follows:

100 times the fraction W/Z,

where W is the number of amino acids scored as identical matches by the sequence alignment program in that program's alignment of C and D, and where Z is the total number of amino acids in D. It will be appreciated that where the length of sequence C is not equal to the length of sequence D, the % sequence identity of C to D will not equal the % sequence identity of D to C.

### Composition

In some embodiments, the disclosure provides a preparation, comprising a recombinant TMEM2 protein or fragment thereof as disclosed and described herein. In some embodiments, the disclosure provides a preparation, comprising a recombinant protein or fragment thereof as disclosed and described herein. In some embodiments, the preparation, further comprises a buffer. In some embodiments, the preparation has >95% purity as assessed by Coomassie stained SDS PAGE.

In some embodiments, the disclosure provides a pharmaceutical composition, comprising a recombinant TMEM2 protein or fragment thereof as disclosed and described herein. In some embodiments, the pharmaceutical composition further comprises a pharmaceutically acceptable excipient. In some embodiments, the pharmaceutical composition further comprises a pharmaceutically active agent. In some embodiments, the pharmaceutical composition further comprises a stabilizing solution. In some embodiments, the stabilizing solution further comprises a carrier selected from the group comprising albumin, detergent, or a surfactant.

In some embodiments, the pharmaceutically active agent is selected from the group consisting of a recombinant TMEM2 protein or fragment thereof comprising at least two, three, four, five, six, seven, eight, nine, ten, 11, 12, 13, 14, 15, 16, 17, 18, or 19 sequences of amino acids set in any of SEQ ID NOs:1, 3, 10, 20-148, 150, or 152. In some embodiments, the pharmaceutically active agent is selected from the group consisting of a recombinant protein or fragment thereof comprising at least two, three, four, five, six, seven, eight, nine, ten, 11, 12, 13, 14, 15, 16, 17, 18, or 19 sequences of amino acids set in any of SEQ ID NOs. 1, 10, 20-148, 150, or 152.

In some embodiments, the pharmaceutical composition further comprises additional active pharmaceutical agents. Non-limiting examples include, antibodies (e.g., a monoclonal antibody, e.g., a sweeping antibody, a broadly neutralizing antibody), immune globulin infusion, an antibody fragment (e.g., neonatal Fc receptor blocker, e.g., efgartigimod), a vaccine (e.g., an anti-HIV vaccine, e.g., CAP256V2LS), a small molecule inhibitor (e.g., a non-nucleoside reverse transcriptase inhibitor, e.g., Rilpivirine), a recombinant molecule (e.g., a recombinant molecule comprising human IgG combined with human hyaluronidase), an antiviral medicine (e.g., an anti-retroviral medicine, e.g., Cabotegravir), In some embodiments, the monoclonal antibody is an anti-CD38 antibody, e.g., Daratumumab. In some embodiments, the monoclonal antibody is an anti-HER2 antibody, e.g., Trastuzumab, e.g., Pertuzumab. In some embodiments, the monoclonal antibody is an anti-CD20 antibody, e.g., Rituximab, e.g., Ocrelizumab. In some embodiments, the monoclonal antibody is an anti-PD1 antibody, e.g., Nivolumab. In some embodiments, the monoclonal antibody is an anti-PDL1 antibody, e.g., Atezolizumab. In some embodiments, the monoclonal antibody is an anti-TIM3 antibody. In some embodiments, the monoclonal antibody is a LAG3 blocking antibody, e.g., Relatlimab. In some embodiments, the monoclonal antibody is an anti-IGF-1R antibody, e.g., Teprotumumab. In some embodiments, the antibody is a complement inhibitor antibody, e.g., ARGX-117. In some embodiments, the antibody is a broadly neutralizing antibody, e.g., N6LS.

In some embodiments, the pharmaceutical composition further comprises additional pharmaceutical agents. Non-limiting examples include, a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition may contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents. Formulations of pharmaceutically therapeutically active compounds and derivatives thereof are provided for administration to humans and animals in unit dosage forms or multiple dosage forms. Generally, dosage forms or compositions containing active ingredient in the range of 0.005% to 100% with the balance made up from non-toxic carrier may be prepared.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

As will be appreciated by those of skill in the art, minor changes can be made to such compositions as described and illustrated herein without substantially eliminating or in some cases without substantially reducing (or potentially even improving) their useful activity and can thus be similarly employed in various applications as described herein.

### Nucleic Acid Molecule

In one aspect, the disclosure provides a nucleic acid molecule, comprising a sequence of nucleotides that encodes a recombinant TMEM2 protein or fragment thereof as disclosed and described herein.

Some embodiments provide a nucleic acid molecule, comprising a sequence of nucleotides selected from the group consisting of: (a) a sequence of nucleotides as provided in Table 2; (b) a sequence of nucleotides set forth in SEQ ID NO. 2; (c) a sequence of nucleotides set forth in SEQ ID NO. 11; (d) a sequence of nucleotides a sequence of nucleotides set forth in SEQ ID No. 149; (e) a sequence of nucleotides a sequence of nucleotides set forth in SEQ ID NO. 153; (f) a sequence that hybridizes under high stringency to at least about 60%, 70%, 80%, 90%, 95%, 98%, 99%, or 100% of the full-length to the sequence of nucleotides set forth in SEQ ID NO. 2; (g) a sequence that hybridizes under high stringency to at least about 60%, 70%, 80%, 90%, 95%, 98%, 99%, or 100% of the full-length to the sequence of nucleotides set forth in SEQ ID NO. 11; (h) a sequence that hybridizes under high stringency to at least about 60%, 70%, 80%, 90%, 95%, 98%, 99%, or 100% of the full-length to the sequence of nucleotides set forth in SEQ ID NO. 149; ((i) a sequence that hybridizes under high stringency to at least about 60%, 70%, 80%, 90%, 95%, 98%, 99%, or 100% of the full-length to the sequence of nucleotides set forth in SEQ ID NO. 153; (i) a sequence that hybridizes under high stringency to at least about 60%, 70%, 80%, or 90% of the full-length to the sequence of nucleotides set forth in SEQ ID NO. 4; (k) a sequence of nucleotides that encodes an TMEM2 extracellular domain or a catalytically active portion thereof that includes a sequence of nucleotides having at least about 60%, 70%, 80%, 90%,95%, 97%, 98%, 99% or 100% sequence identity the sequence set forth in SEQ ID NO.: 2, 11, 149, or 153; (1) a sequence of nucleotides that encodes an TMEM2 extracellular domain or a catalytically active portion thereof that includes a sequence of nucleotides having at least about 60%, 70%, 80%, or 90% sequence identity the sequence set forth in SEQ ID NO. 4; (m) a sequence that is a splice variant of (a), (b) (c), (d), (e), (f), (g), (h), (i), (j), (k), (l), (m), or (n); and (n) a sequence comprising degenerate codons of (a), (b), (c) (d), (e), (f), (g), (h), (i), (j), (k), (l), or (m).

TABLE 2
Exemplary Nucleotide sequences

| | | SEQ ID |
|---|---|---|
| Description | Sequence | NO: |
| Nucleotide | TCCTCAAAATATGCTCCAGATGAAAATTGCCCAGATCAAA | 2 |
| sequence of | ATCCTCGTCTCAGGAATTGGGATCCAGGACAAGATTCTGC | |
| Human | AAAGCAAGTTGTTATCAAGGAGGGAGATATGCTCCGTCT | |
| TMEM2 | GACCTCAGACGCCACCGTGCATTCTATAGTCATTCAGGAT | |
| extracellular | GGAGGACTGCTTGTATTTGGGGACAATAAAGATGGATCC | |
| domain | AGAAATATTACTTTGAGGACTCATTACATCCTGATCCAGG | |
| | ATGGTGGGGCGCTTCATATTGGAGCAGAAAAATGCCGCT | |
| | ATAAATCCAAAGCGACAATTACCTTGTATGGCAAGTCAG | |
| | ATGAAGGTGAAAGTATGCCAACATTTGGCAAAAAGTTTA | |
| | TTGGTGTGGAAGCTGGCGGGACACTGGAGTTACATGGGG | |
| | CACGGAAGGCATCGTGGACGTTGTTGGCAAGGACCCTGA | |
| | ATTCCTCAGGCTTGCCCTTTGGGTCCTATACCTTTGAAAA | |
| | GGACTTTTCCCGGGGCCTCAATGTGAGGGTCATTGACCAA | |
| | GACACGGCCAAAATTTTGGAAAGTGAGAGATTTGATACC | |
| | CATGAATACCGCAATGAGAGCAGGCGGCTTCAGGAGTTT | |
| | CTGAGATTCCAGGATCCAGGTCGGATTGTTGCCATAGCTG | |
| | TCGGGGATTCAGCCGCTAAAAGTCTCTTACAAGGAACCAT | |
| | CCAGATGATCCAGGAACGGTTGGGAAGTGAACTGATCCA | |
| | AGGACTGGGCTACAGGCAAGCTTGGGCTTTAGTTGGTGTC | |
| | ATTGATGGTGGAAGCACTTCTTGCAATGAATCCGTGAGAA | |
| | ACTATGAAAATCATAGCAGTGGCGGGAAGGCTCTTGCCC | |
| | AAAGAGAATTTTATACTGTGGATGGCCAGAAGTTCTCTGT | |
| | GACAGCTTATAGTGAATGGATTGAAGGCGTTTCTCTTTCA | |
| | GGATTCCGGGTAGAGGTTGTAGATGGAGTGAAGCTAAAT | |
| | TTGCTAGATGATGTTAGTAGTTGGAAACCTGGAGACCAG | |
| | ATTGTGGTCGCAAGCACAGACTATTCCATGTACCAAGCAG | |
| | AGGAGTTCACTCTTCTTCCCTGTTCTGAATGCAGCCATTTT | |
| | CAGGTCAAAGTCAAAGAAACCCCTCAGTTCCTGCACATG | |
| | GGTGAGATCATAGACGGTGTAGACATGAGAGCTGAGGTT | |
| | GGAATTCTTACCCGGAATATTGTGATCCAAGGAGAAGTG | |
| | GAGGACTCATGCTACGCAGAAAATCAGTGCCAATTTTTTG | |
| | ATTATGATACCTTTGGGGGACACATTATGATAATGAAAAA | |
| | TTTTACTTCAGTCCATCTTTCTTATGTGGAATTGAAACACA | |
| | TGGGTCAGCAGCAGATGGGGCGATACCCTGTTCATTTTCA | |
| | CCTGTGTGGTGACGTGGATTATAAAGGAGGATACAGACA | |
| | TGCAACATTTGTGGACGGCCTGTCTATTCATCACAGCTTC | |
| | TCAAGGTGCATCACTGTGCATGGGACAAATGGCTTGCTAA | |
| | TAAAAGACACCATTGGGTTTGACACACTAGGTCATTGTTT | |
| | CTTTTTGGAAGATGGTATTGAACAGAGGAATACTTTGTTC | |
| | CACAATCTGGGACTCCTCACCAAGCCGGGTACTCTCCTGC | |
| | CCACCGATAGGAACAACTCCATGTGTACCACCATGCGAG | |
| | ATAAAGTGTTTGGAAATTACATTCCTGTGCCTGCTACTGA | |
| | CTGTATGGCTGTTTCAACTTTCTGGATTGCTCATCCCAACA | |
| | ATAATCTGATTAATAATGCAGCTGCAGGCTCACAGGATGC | |
| | TGGAATATGGTATTTATTCCACAAGGAACCAACTGGGGA | |
| | ATCCAGTGGATTGCAGCTCTTGGCAAAACCAGAACTCACT | |
| | CCATTGGGTATATTTTATAACAACAGGGTCCATTCAAATT | |
| | TTAAGGCTGGCTTATTTATTGACAAAGGTGTCAAAACAAC | |
| | CAACTCTAGTGCTGCTGACCCAAGGGAATACCTCTGTTTG | |
| | GACAATAGTGCAAGATTTCGACCTCATCAGGATGCAAAC | |
| | CCCGAAAAACCACGTGTTGCTGCTCTAATTGACAGGCTCA | |
| | TTGCTTTTAAAAATAATGATAATGGAGCTTGGGTCAGAGG | |
| | AGGAGATATTATCGTTCAAAATTCAGCATTTGCAGATAAT | |
| | GGAATAGGACTGACCTTTGCCAGTGATGGAAGCTTCCCA | |
| | AGTGATGAAGGTTCCAGCCAAGAGGTATCTGAATCTCTCT | |
| | TTGTTGGGGAGAGCAGGAATTACGGCTTTCAGGGTGGTC | |
| | AGAACAAGTATGTAGGCACTGGAGGAATAGACCAGAAGC | |
| | CTCGAACATTACCCAGGAACAGGACGTTCCCAATTAGAG | |
| | GCTTTCAGATTTATGATGGGCCCATTCATCTCACAAGGAG | |
| | CACTTTCAAAAAATATGTGCCAACTCCAGATAGGTACAGC | |
| | AGTGCAATTGGCTTCCTCATGAAGAATTCCTGGCAGATAA | |
| | CCCCCAGGAATAATATCTCCCTCGTGAAGTTTGGTCCACA | |
| | TGTCTCTCTGAATGTCTTTTTTGGAAAGCCTGGTCCCTGGT | |
| | TTGAAGATTGTGAGATGGATGGTGATAAGAACTCCATATT | |
| | CCATGACATTGATGGCTCTGTGACAGGATACAAGGATGCT | |
| | TATGTGGGAAGAATGGACAACTACCTGATCCGCCATCCA | |
| | AGCTGTGTAAATGTGTCTAAGTGGAATGCAGTGATCTGCA | |
| | GTGGGACCTATGCACAGGTCTATGTACAGACATGGAGCA | |
| | CTCAGAATCTTTCTATGACCATTACACGAGATGAGTATCC | |
| | GTCCAACCCTATGGTGCTCCGAGGTATTAATCAGAAGGCT | |
| | GCCTTTCCACAGTACCAGCCTGTCGTCATGCTGGAGAAGG | |
| | GTTATACCATCCACTGGAATGGGCCGGCACCACGGACTA | |
| | CATTTCTATACCTCGTCAACTTCAACAAGAATGACTGGAT | |
| | TCGAGTTGGCCTTTGCTATCCATCAAACACAAGTTTTCAA | |
| | GTTACCTTTGGCTATTTGCAGCGGCAGAATGGCTCATTAT | |
| | CCAAAATCGAAGAATATGAGCCTGTGCATTCACTGGAAG | |
| | AACTGCAAAGAAAGCAATCCGAGAGGAAATTCTATTTTG | |
| | ACTCCAGCACGGGGTTACTGTTTTTGTATCTCAAAGCCAA | |
| | AAGCCACAGGCATGGCCACAGTTACTGTTCATCTCAGGG | |
| | ATGTGAAAGAGTCAAGATCCAAGCAGCCACAGACTCAAA | |
| | GGACATCAGTAACTGCATGGCCAAAGCATACCCACAGTA | |
| | CTACAGAAAGCCGTCAGTGGTCAAGCGGATGCCGGCCAT | |
| | GCTCACTGGACTCTGTCAAGGCTGTGGCACTCGGCAGGTG | |
| | GTGTTTACTAGTGATCCTCATAAAAGTTACCTCCCTGTGC | |
| | AATTCCAGTCACCTGATAAAGCAGAAACCCAGCGTGGAG | |
| | ACCCGTCTGTTATTTCTGTCAATGGCACTGACTTTACCTTC | |
| | CGAAGTGCAGGCGTCCTCCTCCTTGTTGTGGATCCGTGCA | |
| | GCGTTCCATTCCGCTTGACGGAAAAAACGGTTTTTCCTCT | |
| | TGCTGATGTCAGTCGCATTGAAGAGTATTTAAAAACAGGC | |
| | ATCCCTCCAAGGTCCATTGTTCTGTTGAGCACAAGAGGAG | |
| | AAATAAAGCAGTTAAACATTTCACACTTACTAGTACCTCT | |
| | GGGATTAGCCAAACCAGCTCATCTTTATGACAAAGGGAG | |
| | TACCATATTTTTGGGATTCAGTGGAAACTTTAAACCATCA | |
| | TGGACTAAGCTATTTACCAGTCCTGCTGGACAGGGCCTTG | |
| | GGGTGCTTGAACAATTCATACCTTTGCAGCTGGACGAATA | |
| | TGGTTGTCCCAGAGCCACCACTGTCCGCAGAAGAGACCT | |
| | GGAACTGCTAAAGCAAGCTTCAAAAGCACAT | |
| Nucleotide | AGTTGAGTACGAGCGGCAGCTGCGGGCGCCGGCAATCGA | 4 |
| sequence of | GTGGCTTCCTGGCCATGGAGAGGAGATCTGGACCCGAGG | |
| Human | ACGCCAGGGACTCCTCTCCGTCTCCCTGGACCGCTGTGTA | |
| TMEM2 | GGCACCTGAGTTAACGGACGCTAAGCGGGGTAGGGGAAG | |
| Isoform a | TAACAGTGTATCATGTATGCCACTGATTCCAGGGGACACT | |
| | CCCCTGCTTTCCTCCAACCTCAGAATGGAAATAGTCGTCA | |
| | CCCATCTGGCTATGTTCCAGGGAAGGTTGTCCCATTGCGT | |
| | CCCCCTCCTCCTCCAAAGAGTCAAGCTTCAGCCAAATTTA | |
| | CCTCCATCAGACGAGAAGACCGGGCAACCTTCGCATTCTC | |
| | ACCTGAAGAACAGCAAGCCCAGAGAGAAAGTCAAAAGC | |
| | AAAAGAGACACAAAAATACTTTCATTTGTTTTGCTATTAC | |
| | TAGTTTCTCATTTTTTATTGCACTTGCAATCATTTTAGGAA | |
| | TATCCTCAAAATATGCTCCAGATGAAAATTGCCCAGATCA | |
| | AAATCCTCGTCTCAGGAATTGGGATCCAGGACAAGATTCT | |
| | GCAAAGCAAGTTGTTATCAAGGAGGGAGATATGCTCCGT | |
| | CTGACCTCAGACGCCACCGTGCATTCTATAGTCATTCAGG | |
| | ATGGAGGACTGCTTGTATTTGGGGACAATAAAGATGGAT | |
| | CCAGAAATATTACTTTGAGGACTCATTACATCCTGATCCA | |
| | GGATGGTGGGGCGCTTCATATTGGAGCAGAAAAATGCCG | |
| | CTATAAATCCAAAGCGACAATTACCTTGTATGGCAAGTCA | |
| | GATGAAGGTGAAAGTATGCCAACATTTGGCAAAAAGTTT | |
| | ATTGGTGTGGAAGCTGGCGGGACACTGGAGTTACATGGG | |
| | GCACGGAAGGCATCGTGGACGTTGTTGGCAAGGACCCTG | |
| | AATTCCTCAGGCTTGCCCTTTGGGTCCTATACCTTTGAAA | |
| | AGGACTTTTCCCGGGGCCTCAATGTGAGGGTCATTGACCA | |
| | AGACACGGCCAAAATTTTGGAAAGTGAGAGATTTGATAC | |
| | CCATGAATACCGCAATGAGAGCAGGCGGCTTCAGGAGTT | |
| | TCTGAGATTCCAGGATCCAGGTCGGATTGTTGCCATAGCT | |
| | GTCGGGGATTCAGCCGCTAAAAGTCTCTTACAAGGAACC | |
| | ATCCAGATGATCCAGGAACGGTTGGGAAGTGAACTGATC | |
| | CAAGGACTGGGCTACAGGCAAGCTTGGGCTTTAGTTGGT | |
| | GTCATTGATGGTGGAAGCACTTCTTGCAATGAATCCGTGA | |
| | GAAACTATGAAAATCATAGCAGTGGCGGGAAGGCTCTTG | |
| | CCCAAAGAGAATTTTATACTGTGGATGGCCAGAAGTTCTC | |
| | TGTGACAGCTTATAGTGAATGGATTGAAGGCGTTTCTCTT | |
| | TCAGGATTCCGGGTAGAGGTTGTAGATGGAGTGAAGCTA | |
| | AATTTGCTAGATGATGTTAGTAGTTGGAAACCTGGAGACC | |
| | AGATTGTGGTCGCAAGCACAGACTATTCCATGTACCAAGC | |
| | AGAGGAGTTCACTCTTCTTCCCTGTTCTGAATGCAGCCAT | |
| | TTTCAGGTCAAAGTCAAAGAAACCCCTCAGTTCCTGCACA | |
| | TGGGTGAGATCATAGACGGTGTAGACATGAGAGCTGAGG | |
| | TTGGAATTCTTACCCGGAATATTGTGATCCAAGGAGAAGT | |
| | GGAGGACTCATGCTACGCAGAAAATCAGTGCCAATTTTTT | |
| | GATTATGATACCTTTGGGGGACACATTATGATAATGAAAA | |
| | ATTTTACTTCAGTCCATCTTTCTTATGTGGAATTGAAACAC | |
| | ATGGGTCAGCAGCAGATGGGGCGATACCCTGTTCATTTTC | |
| | ACCTGTGTGGTGACGTGGATTATAAAGGAGGATACAGAC | |
| | ATGCAACATTTGTGGACGGCCTGTCTATTCATCACAGCTT | |
| | CTCAAGGTGCATCACTGTGCATGGGACAAATGGCTTGCTA | |
| | ATAAAAGACACCATTGGGTTTGACACACTAGGTCATTGTT | |
| | TCTTTTTGGAAGATGGTATTGAACAGAGGAATACTTTGTT | |
| | CCACAATCTGGGACTCCTCACCAAGCCGGGTACTCTCCTG | |
| | CCCACCGATAGGAACAACTCCATGTGTACCACCATGCGA | |
| | GATAAAGTGTTTGGAAATTACATTCCTGTGCCTGCTACTG | |
| | ACTGTATGGCTGTTTCAACTTTCTGGATTGCTCATCCCAAC | |
| | AATAATCTGATTAATAATGCAGCTGCAGGCTCACAGGAT | |
| | GCTGGAATATGGTATTTATTCCACAAGGAACCAACTGGG | |
| | GAATCCAGTGGATTGCAGCTCTTGGCAAAACCAGAACTC | |
| | ACTCCATTGGGTATATTTTATAACAACAGGGTCCATTCAA | |
| | ATTTTAAGGCTGGCTTATTTATTGACAAAGGTGTCAAAAC | |
| | AACCAACTCTAGTGCTGCTGACCCAAGGGAATACCTCTGT | |
| | TTGGACAATAGTGCAAGATTTCGACCTCATCAGGATGCAA | |
| | ACCCCGAAAAACCACGTGTTGCTGCTCTAATTGACAGGCT | |
| | CATTGCTTTTAAAAATAATGATAATGGAGCTTGGGTCAGA | |
| | GGAGGAGATATTATCGTTCAAAATTCAGCATTTGCAGATA | |
| | ATGGAATAGGACTGACCTTTGCCAGTGATGGAAGCTTCCC | |
| | AAGTGATGAAGGTTCCAGCCAAGAGGTATCTGAATCTCTC | |
| | TTTGTTGGGGAGAGCAGGAATTACGGCTTTCAGGGTGGTC | |
| | AGAACAAGTATGTAGGCACTGGAGGAATAGACCAGAAGC | |
| | CTCGAACATTACCCAGGAACAGGACGTTCCCAATTAGAG | |
| | GCTTTCAGATTTATGATGGGCCCATTCATCTCACAAGGAG | |
| | CACTTTCAAAAAATATGTGCCAACTCCAGATAGGTACAGC | |
| | AGTGCAATTGGCTTCCTCATGAAGAATTCCTGGCAGATAA | |
| | CCCCCAGGAATAATATCTCCCTCGTGAAGTTTGGTCCACA | |
| | TGTCTCTCTGAATGTCTTTTTTGGAAAGCCTGGTCCCTGGT | |
| | TTGAAGATTGTGAGATGGATGGTGATAAGAACTCCATATT | |
| | CCATGACATTGATGGCTCTGTGACAGGATACAAGGATGCT | |
| | TATGTGGGAAGAATGGACAACTACCTGATCCGCCATCCA | |
| | AGCTGTGTAAATGTGTCTAAGTGGAATGCAGTGATCTGCA | |
| | GTGGGACCTATGCACAGGTCTATGTACAGACATGGAGCA | |
| | CTCAGAATCTTTCTATGACCATTACACGAGATGAGTATCC | |
| | GTCCAACCCTATGGTGCTCCGAGGTATTAATCAGAAGGCT | |
| | GCCTTTCCACAGTACCAGCCTGTCGTCATGCTGGAGAAGG | |
| | GTTATACCATCCACTGGAATGGGCCGGCACCACGGACTA | |
| | CATTTCTATACCTCGTCAACTTCAACAAGAATGACTGGAT | |
| | TCGAGTTGGCCTTTGCTATCCATCAAACACAAGTTTTCAA | |
| | GTTACCTTTGGCTATTTGCAGCGGCAGAATGGCTCATTAT | |
| | CCAAAATCGAAGAATATGAGCCTGTGCATTCACTGGAAG | |
| | AACTGCAAAGAAAGCAATCCGAGAGGAAATTCTATTTTG | |
| | ACTCCAGCACGGGGTTACTGTTTTTGTATCTCAAAGCCAA | |
| | AAGCCACAGGCATGGCCACAGTTACTGTTCATCTCAGGG | |
| | ATGTGAAAGAGTCAAGATCCAAGCAGCCACAGACTCAAA | |
| | GGACATCAGTAACTGCATGGCCAAAGCATACCCACAGTA | |
| | CTACAGAAAGCCGTCAGTGGTCAAGCGGATGCCGGCCAT | |
| | GCTCACTGGACTCTGTCAAGGCTGTGGCACTCGGCAGGTG | |
| | GTGTTTACTAGTGATCCTCATAAAAGTTACCTCCCTGTGC | |
| | AATTCCAGTCACCTGATAAAGCAGAAACCCAGCGTGGAG | |
| | ACCCGTCTGTTATTTCTGTCAATGGCACTGACTTTACCTTC | |
| | CGAAGTGCAGGCGTCCTCCTCCTTGTTGTGGATCCGTGCA | |
| | GCGTTCCATTCCGCTTGACGGAAAAAACGGTTTTTCCTCT | |
| | TGCTGATGTCAGTCGCATTGAAGAGTATTTAAAAACAGGC | |
| | ATCCCTCCAAGGTCCATTGTTCTGTTGAGCACAAGAGGAG | |
| | AAATAAAGCAGTTAAACATTTCACACTTACTAGTACCTCT | |
| | GGGATTAGCCAAACCAGCTCATCTTTATGACAAAGGGAG | |
| | TACCATATTTTTGGGATTCAGTGGAAACTTTAAACCATCA | |
| | TGGACTAAGCTATTTACCAGTCCTGCTGGACAGGGCCTTG | |
| | GGGTGCTTGAACAATTCATACCTTTGCAGCTGGACGAATA | |
| | TGGTTGTCCCAGAGCCACCACTGTCCGCAGAAGAGACCT | |
| | GGAACTGCTAAAGCAAGCTTCAAAAGCACATTAGAGACT | |
| | AACTGTAACTTAAGTGCTGGGGGAAAAAAAATGTGAACT | |
| | AACTTATTTAATTTATGGCATTTTAAAATGACACTGTTAA | |
| | CCCAACGGAACCATTTTCCAGTTTGATACAGAATGGGGA | |
| | GAAAAGAAAGCGTTTGAAATTATTGCTTGGATACCAGCTT | |
| | CATGCACCTTCTAGTTGTACAAAATGTTAAAGACGTTGTT | |
| | TGTATTTGTAAGGCTGGTGTATTCAGAGAGCAGATCTCTT | |
| | ATTCCTCACTTTCCACCCCCGTATTTTGTAATGACCATGAG | |
| | CAATGTTTTTACTTTTTGTATAATGGGGTGGGGTGGAGTG | |
| | GGGGCTTCTGAGAGTCAGCCTGAGGTCTTTAGAGGACCA | |
| | GCTATTGTAGCACCTTGGATACTTGAAGTTTAATGCTCAG | |
| | TTGGGTCGGGTGGCAGTTGACTTGGTGGCTGGCATGTTCA | |
| | GCAGTGCCTGGGGCCCTGTTTCTGGGCAGCCTTTGAGGAT | |
| | TTTCTATGATATTGAATGACAGTTTTAAGTGGCAACTCAG | |
| | GCCCAGCTCATGCCCTTTTTTGCCTGGACATGTGCTATTTT | |
| | TATTCACTTATATATCAATTACTTGTAAGGGTTAAACTTTC | |
| | AAACAGGAAGTATATTGGGACAAAAGGGCTCTTGGGGAT | |
| | TAGATATCCCTTTAATCTGTGACCATTGGGCAAAAAATTT | |
| | TCCTGCAGCAAAAGTCTGAGGCTGTTGGGACCATTTTTGC | |
| | AGCTTTAATCCTTAGCCTCTTTTGACTGTATATTTGTGTTT | |
| | AAAATGCAGAGCTCAACTGAATATTTCCTTTTTTGTTTTGT | |
| | TTTGTTTTGTTTTAAGAAGTAGGTTGTTTTCCTGAACCGTA | |
| | AACTTGTATCATTTTAACTTGCACAAAGGAAGTCTGTTCT | |
| | TGGTATTGCTCTTGCACTTGGGTTTTTTGTTATTGTTTTGT | |
| | GTGGATTTTTTAAAGCTTTTCTGTTCACCCTCCTGCCAGGA | |
| | AAATCCCAGAAAGCTTAATGATACCCCAAAATGATTACA | |
| | CCCAGGGAGGAAAAAAAGGAGCGCTTTCTAGGGTCAGAA | |
| | TCGTGGAGAGAATACTCAGAAATGAACCTCTTTAAAGCCT | |
| | TGCAGGAATGAGTCACTCTTACTTAATGAAATGTTAAAGC | |
| | CAATTAAAAAGCATGCTGTGATGCCCAGCTTCCCTTTCCA | |
| | CAGGGTGCATGCGTCTCCTGCTGGTGAATCACATGCGGCA | |
| | AGAGGCAACTGGCTCCACAGCCTGGGATGCTGCCGTACC | |
| | AAGAGGAAAGAAGCAGCAAAATGCCTTTACGTTGTTCTA | |
| | AACCCCCGACGCATAAAGTGTAGAGGAGGGATGGCCAAG | |
| | GGTGGGTGGTAGAAAGTGTGTTCAGGCTGACACTGGCAA | |
| | TGAGTACAGATAATTTCACTTTCCTCTTCTAGGGGCAAAG | |
| | GCTGATGGCCTCTACCTTTGTATCCAGGAGAAACTGCAGA | |
| | GCAGCCCTGTGACTTTACAAAATATGCTACCTCAAAGTGC | |
| | TACCGATAAACCTTTCTAATTGTAAGTGCCCTTACTAAGG | |
| | GCACATGTCTTAATCAAAGTTAGTTTTTTGTTTTCTGGTTT | |
| | GTTTTTTTTTTTTGTATATTGATGAATGAGATCTTACCTAT | |
| | TAAATATATTATTGGATTATGGTTCCTGAAGGTCATTAGA | |
| | GTGTGTGTGTGTGTGTGTGTGTGTGTGTGTGTTTTATGACT | |
| | TAAATATCTTTACGTGTGTTTTTTAGAGCTTGGTTCTTTAA | |
| | AGATTTGGAGAAGATATGTAAATTACCAAGGCACTTGGTT | |
| | TTTCTGTTTTATATACTAATAATCAGGGCCTAAGTTAAAT | |
| | AAAAATATGTGTGCATGTATTTTA | |
| Nucleotide | ATGGAGACAGACACACTCCTGCTATGGGTACTGCTGCTCT | 11 |
| sequence of | GGGTTCCAGGTTCCACTGGTGACGCGGCCCAGCCGGCCA | |
| Recombinant | GGCGCGCGCGCCGTACGAAGCTTGGTACCTCCTCAAAAT | |
| protein | ATGCTCCAGATGAAAATTGCCCAGATCAAAATCCTCGTCT | |
| (Human | CAGGAATTGGGATCCAGGACAAGATTCTGCAAAGCAAGT | |
| TMEM2 | TGTTATCAAGGAGGGAGATATGCTCCGTCTGACCTCAGAC | |
| extracellular | GCCACCGTGCATTCTATAGTCATTCAGGATGGAGGACTGC | |
| domain with | TTGTATTTGGGGACAATAAAGATGGATCCAGAAATATTAC | |
| sequences | TTTGAGGACTCATTACATCCTGATCCAGGATGGTGGGGCG | |
| remaining at | CTTCATATTGGAGCAGAAAAATGCCGCTATAAATCCAAA | |
| N-terminus | GCGACAATTACCTTGTATGGCAAGTCAGATGAAGGTGAA | |
| and C- | AGTATGCCAACATTTGGCAAAAAGTTTATTGGTGTGGAAG | |
| terminus) | CTGGCGGGACACTGGAGTTACATGGGGCACGGAAGGCAT | |
| | CGTGGACGTTGTTGGCAAGGACCCTGAATTCCTCAGGCTT | |
| | GCCCTTTGGGTCCTATACCTTTGAAAAGGACTTTTCCCGG | |
| | GGCCTCAATGTGAGGGTCATTGACCAAGACACGGCCAAA | |
| | ATTTTGGAAAGTGAGAGATTTGATACCCATGAATACCGCA | |
| | ATGAGAGCAGGCGGCTTCAGGAGTTTCTGAGATTCCAGG | |
| | ATCCAGGTCGGATTGTTGCCATAGCTGTCGGGGATTCAGC | |
| | CGCTAAAAGTCTCTTACAAGGAACCATCCAGATGATCCA | |
| | GGAACGGTTGGGAAGTGAACTGATCCAAGGACTGGGCTA | |
| | CAGGCAAGCTTGGGCTTTAGTTGGTGTCATTGATGGTGGA | |
| | AGCACTTCTTGCAATGAATCCGTGAGAAACTATGAAAATC | |
| | ATAGCAGTGGCGGGAAGGCTCTTGCCCAAAGAGAATTTT | |
| | ATACTGTGGATGGCCAGAAGTTCTCTGTGACAGCTTATAG | |
| | TGAATGGATTGAAGGCGTTTCTCTTTCAGGATTCCGGGTA | |
| | GAGGTTGTAGATGGAGTGAAGCTAAATTTGCTAGATGAT | |
| | GTTAGTAGTTGGAAACCTGGAGACCAGATTGTGGTCGCA | |
| | AGCACAGACTATTCCATGTACCAAGCAGAGGAGTTCACT | |
| | CTTCTTCCCTGTTCTGAATGCAGCCATTTTCAGGTCAAAGT | |
| | CAAAGAAACCCCTCAGTTCCTGCACATGGGTGAGATCAT | |
| | AGACGGTGTAGACATGAGAGCTGAGGTTGGAATTCTTAC | |
| | CCGGAATATTGTGATCCAAGGAGAAGTGGAGGACTCATG | |
| | CTACGCAGAAAATCAGTGCCAATTTTTTGATTATGATACC | |
| | TTTGGGGGACACATTATGATAATGAAAAATTTTACTTCAG | |
| | TCCATCTTTCTTATGTGGAATTGAAACACATGGGTCAGCA | |
| | GCAGATGGGGCGATACCCTGTTCATTTTCACCTGTGTGGT | |
| | GACGTGGATTATAAAGGAGGATACAGACATGCAACATTT | |
| | GTGGACGGCCTGTCTATTCATCACAGCTTCTCAAGGTGCA | |
| | TCACTGTGCATGGGACAAATGGCTTGCTAATAAAAGACA | |
| | CCATTGGGTTTGACACACTAGGTCATTGTTTCTTTTTGGAA | |
| | GATGGTATTGAACAGAGGAATACTTTGTTCCACAATCTGG | |
| | GACTCCTCACCAAGCCGGGTACTCTCCTGCCCACCGATAG | |
| | GAACAACTCCATGTGTACCACCATGCGAGATAAAGTGTTT | |
| | GGAAATTACATTCCTGTGCCTGCTACTGACTGTATGGCTG | |
| | TTTCAACTTTCTGGATTGCTCATCCCAACAATAATCTGATT | |
| | AATAATGCAGCTGCAGGCTCACAGGATGCTGGAATATGG | |
| | TATTTATTCCACAAGGAACCAACTGGGGAATCCAGTGGAT | |
| | TGCAGCTCTTGGCAAAACCAGAACTCACTCCATTGGGTAT | |
| | ATTTTATAACAACAGGGTCCATTCAAATTTTAAGGCTGGC | |
| | TTATTTATTGACAAAGGTGTCAAAACAACCAACTCTAGTG | |
| | CTGCTGACCCAAGGGAATACCTCTGTTTGGACAATAGTGC | |
| | AAGATTTCGACCTCATCAGGATGCAAACCCCGAAAAACC | |
| | ACGTGTTGCTGCTCTAATTGACAGGCTCATTGCTTTTAAA | |
| | AATAATGATAATGGAGCTTGGGTCAGAGGAGGAGATATT | |
| | ATCGTTCAAAATTCAGCATTTGCAGATAATGGAATAGGAC | |
| | TGACCTTTGCCAGTGATGGAAGCTTCCCAAGTGATGAAGG | |
| | TTCCAGCCAAGAGGTATCTGAATCTCTCTTTGTTGGGGAG | |
| | AGCAGGAATTACGGCTTTCAGGGTGGTCAGAACAAGTAT | |
| | GTAGGCACTGGAGGAATAGACCAGAAGCCTCGAACATTA | |
| | CCCAGGAACAGGACGTTCCCAATTAGAGGCTTTCAGATTT | |
| | ATGATGGGCCCATTCATCTCACAAGGAGCACTTTCAAAAA | |
| | ATATGTGCCAACTCCAGATAGGTACAGCAGTGCAATTGG | |
| | CTTCCTCATGAAGAATTCCTGGCAGATAACCCCCAGGAAT | |
| | AATATCTCCCTCGTGAAGTTTGGTCCACATGTCTCTCTGA | |
| | ATGTCTTTTTTGGAAAGCCTGGTCCCTGGTTTGAAGATTG | |
| | TGAGATGGATGGTGATAAGAACTCCATATTCCATGACATT | |
| | GATGGCTCTGTGACAGGATACAAGGATGCTTATGTGGGA | |
| | AGAATGGACAACTACCTGATCCGCCATCCAAGCTGTGTA | |
| | AATGTGTCTAAGTGGAATGCAGTGATCTGCAGTGGGACCT | |
| | ATGCACAGGTCTATGTACAGACATGGAGCACTCAGAATC | |
| | TTTCTATGACCATTACACGAGATGAGTATCCGTCCAACCC | |
| | TATGGTGCTCCGAGGTATTAATCAGAAGGCTGCCTTTCCA | |
| | CAGTACCAGCCTGTCGTCATGCTGGAGAAGGGTTATACCA | |
| | TCCACTGGAATGGGCCGGCACCACGGACTACATTTCTATA | |
| | CCTCGTCAACTTCAACAAGAATGACTGGATTCGAGTTGGC | |
| | CTTTGCTATCCATCAAACACAAGTTTTCAAGTTACCTTTG | |
| | GCTATTTGCAGCGGCAGAATGGCTCATTATCCAAAATCGA | |
| | AGAATATGAGCCTGTGCATTCACTGGAAGAACTGCAAAG | |
| | AAAGCAATCCGAGAGGAAATTCTATTTTGACTCCAGCAC | |
| | GGGGTTACTGTTTTTGTATCTCAAAGCCAAAAGCCACAGG | |
| | CATGGCCACAGTTACTGTTCATCTCAGGGATGTGAAAGAG | |
| | TCAAGATCCAAGCAGCCACAGACTCAAAGGACATCAGTA | |
| | ACTGCATGGCCAAAGCATACCCACAGTACTACAGAAAGC | |
| | CGTCAGTGGTCAAGCGGATGCCGGCCATGCTCACTGGACT | |
| | CTGTCAAGGCTGTGGCACTCGGCAGGTGGTGTTTACTAGT | |
| | GATCCTCATAAAAGTTACCTCCCTGTGCAATTCCAGTCAC | |
| | CTGATAAAGCAGAAACCCAGCGTGGAGACCCGTCTGTTA | |
| | TTTCTGTCAATGGCACTGACTTTACCTTCCGAAGTGCAGG | |
| | CGTCCTCCTCCTTGTTGTGGATCCGTGCAGCGTTCCATTCC | |
| | GCTTGACGGAAAAAACGGTTTTTCCTCTTGCTGATGTCAG | |
| | TCGCATTGAAGAGTATTTAAAAACAGGCATCCCTCCAAG | |
| | GTCCATTGTTCTGTTGAGCACAAGAGGAGAAATAAAGCA | |
| | GTTAAACATTTCACACTTACTAGTACCTCTGGGATTAGCC | |
| | AAACCAGCTCATCTTTATGACAAAGGGAGTACCATATTTT | |
| | TGGGATTCAGTGGAAACTTTAAACCATCATGGACTAAGCT | |
| | ATTTACCAGTCCTGCTGGACAGGGCCTTGGGGTGCTTGAA | |
| | CAATTCATACCTTTGCAGCTGGACGAATATGGTTGTCCCA | |
| | GAGCCACCACTGTCCGCAGAAGAGACCTGGAACTGCTAA | |
| | AGCAAGCTTCAAAAGCACATGGTTCTAGCGAAAACCTGT | |
| | ATTTTCAGGGCTCTAGCCATCATCACCATCACCATTGACT | |
| | CGAG | |
| Nucleotide | ATGGAGACAGACACACTCCTGCTATGGGTACTGCTGCTCTG | 149 |
| sequence of | GGTTCCAGGTTCCACTGGTGACGCGGCCCAGCCGGCCAGG | |
| Recombinant | CGCGCGCGCCGTACGAAGCTTGGTACCTCCTCAAAATATGC | |
| protein | TCCAGATGAAAATTGCCCAGATCAAAATCCTCGTCTCAGGA | |
| (Human | ATTGGGATCCAGGACAAGATTCTGCAAAGCAAGTTGTTATC | |
| TMEM2 | AAGGAGGGAGATATGCTCCGTCTGACCTCAGACGCCACCG | |
| extracellular | TGCATTCTATAGTCATTCAGGATGGAGGACTGCTTGTATTT | |
| domain with | GGGGACAATAAAGATGGATCCAGAAATATTACTTTGAGGA | |
| sequences | CTCATTACATCCTGATCCAGGATGGTGGGGCGCTTCATATT | |
| remaining at | GGAGCAGAAAAATGCCGCTATAAATCCAAAGCGACAATTA | |
| N-terminus | CCTTGTATGGCAAGTCAGATGAAGGTGAAAGTATGCCAAC | |
| and C- | ATTTGGCAAAAAGTTTATTGGTGTGGAAGCTGGCGGGACA | |
| terminus) | CTGGAGTTACATGGGGCACGGAAGGCATCGTGGACGTTGT | |
| without H-tag | TGGCAAGGACCCTGAATTCCTCAGGCTTGCCCTTTGGGTCC | |
| | TATACCTTTGAAAAGGACTTTTCCCGGGGCCTCAATGTGAG | |
| | GGTCATTGACCAAGACACGGCCAAAATTTTGGAAAGTGAG | |
| | AGATTTGATACCCATGAATACCGCAATGAGAGCAGGCGGC | |
| | TTCAGGAGTTTCTGAGATTCCAGGATCCAGGTCGGATTGTT | |
| | GCCATAGCTGTCGGGGATTCAGCCGCTAAAAGTCTCTTACA | |
| | AGGAACCATCCAGATGATCCAGGAACGGTTGGGAAGTGAA | |
| | CTGATCCAAGGACTGGGCTACAGGCAAGCTTGGGCTTTAGT | |
| | TGGTGTCATTGATGGTGGAAGCACTTCTTGCAATGAATCCG | |
| | TGAGAAACTATGAAAATCATAGCAGTGGCGGGAAGGCTCT | |
| | TGCCCAAAGAGAATTTTATACTGTGGATGGCCAGAAGTTCT | |
| | CTGTGACAGCTTATAGTGAATGGATTGAAGGCGTTTCTCTT | |
| | TCAGGATTCCGGGTAGAGGTTGTAGATGGAGTGAAGCTAA | |
| | ATTTGCTAGATGATGTTAGTAGTTGGAAACCTGGAGACCAG | |
| | ATTGTGGTCGCAAGCACAGACTATTCCATGTACCAAGCAG | |
| | AGGAGTTCACTCTTCTTCCCTGTTCTGAATGCAGCCATTTTC | |
| | AGGTCAAAGTCAAAGAAACCCCTCAGTTCCTGCACATGGG | |
| | TGAGATCATAGACGGTGTAGACATGAGAGCTGAGGTTGGA | |
| | ATTCTTACCCGGAATATTGTGATCCAAGGAGAAGTGGAGG | |
| | ACTCATGCTACGCAGAAAATCAGTGCCAATTTTTTGATTAT | |
| | GATACCTTTGGGGGACACATTATGATAATGAAAAATTTTAC | |
| | TTCAGTCCATCTTTCTTATGTGGAATTGAAACACATGGGTC | |
| | AGCAGCAGATGGGGCGATACCCTGTTCATTTTCACCTGTGT | |
| | GGTGACGTGGATTATAAAGGAGGATACAGACATGCAACAT | |
| | TTGTGGACGGCCTGTCTATTCATCACAGCTTCTCAAGGTGC | |
| | ATCACTGTGCATGGGACAAATGGCTTGCTAATAAAAGACA | |
| | CCATTGGGTTTGACACACTAGGTCATTGTTTCTTTTTGGAA | |
| | GATGGTATTGAACAGAGGAATACTTTGTTCCACAATCTGGG | |
| | ACTCCTCACCAAGCCGGGTACTCTCCTGCCCACCGATAGGA | |
| | ACAACTCCATGTGTACCACCATGCGAGATAAAGTGTTTGGA | |
| | AATTACATTCCTGTGCCTGCTACTGACTGTATGGCTGTTTCA | |
| | ACTTTCTGGATTGCTCATCCCAACAATAATCTGATTAATAA | |
| | TGCAGCTGCAGGCTCACAGGATGCTGGAATATGGTATTTAT | |
| | TCCACAAGGAACCAACTGGGGAATCCAGTGGATTGCAGCT | |
| | CTTGGCAAAACCAGAACTCACTCCATTGGGTATATTTTATA | |
| | ACAACAGGGTCCATTCAAATTTTAAGGCTGGCTTATTTATT | |
| | GACAAAGGTGTCAAAACAACCAACTCTAGTGCTGCTGACC | |
| | CAAGGGAATACCTCTGTTTGGACAATAGTGCAAGATTTCGA | |
| | CCTCATCAGGATGCAAACCCCGAAAAACCACGTGTTGCTG | |
| | CTCTAATTGACAGGCTCATTGCTTTTAAAAATAATGATAAT | |
| | GGAGCTTGGGTCAGAGGAGGAGATATTATCGTTCAAAATT | |
| | CAGCATTTGCAGATAATGGAATAGGACTGACCTTTGCCAGT | |
| | GATGGAAGCTTCCCAAGTGATGAAGGTTCCAGCCAAGAGG | |
| | TATCTGAATCTCTCTTTGTTGGGGAGAGCAGGAATTACGGC | |
| | TTTCAGGGTGGTCAGAACAAGTATGTAGGCACTGGAGGAA | |
| | TAGACCAGAAGCCTCGAACATTACCCAGGAACAGGACGTT | |
| | CCCAATTAGAGGCTTTCAGATTTATGATGGGCCCATTCATC | |
| | TCACAAGGAGCACTTTCAAAAAATATGTGCCAACTCCAGA | |
| | TAGGTACAGCAGTGCAATTGGCTTCCTCATGAAGAATTCCT | |
| | GGCAGATAACCCCCAGGAATAATATCTCCCTCGTGAAGTTT | |
| | GGTCCACATGTCTCTCTGAATGTCTTTTTTGGAAAGCCTGG | |
| | TCCCTGGTTTGAAGATTGTGAGATGGATGGTGATAAGAACT | |
| | CCATATTCCATGACATTGATGGCTCTGTGACAGGATACAAG | |
| | GATGCTTATGTGGGAAGAATGGACAACTACCTGATCCGCC | |
| | ATCCAAGCTGTGTAAATGTGTCTAAGTGGAATGCAGTGATC | |
| | TGCAGTGGGACCTATGCACAGGTCTATGTACAGACATGGA | |
| | GCACTCAGAATCTTTCTATGACCATTACACGAGATGAGTAT | |
| | CCGTCCAACCCTATGGTGCTCCGAGGTATTAATCAGAAGGC | |
| | TGCCTTTCCACAGTACCAGCCTGTCGTCATGCTGGAGAAGG | |
| | GTTATACCATCCACTGGAATGGGCCGGCACCACGGACTAC | |
| | ATTTCTATACCTCGTCAACTTCAACAAGAATGACTGGATTC | |
| | GAGTTGGCCTTTGCTATCCATCAAACACAAGTTTTCAAGTT | |
| | ACCTTTGGCTATTTGCAGCGGCAGAATGGCTCATTATCCAA | |
| | AATCGAAGAATATGAGCCTGTGCATTCACTGGAAGAACTG | |
| | CAAAGAAAGCAATCCGAGAGGAAATTCTATTTTGACTCCA | |
| | GCACGGGGTTACTGTTTTTGTATCTCAAAGCCAAAAGCCAC | |
| | AGGCATGGCCACAGTTACTGTTCATCTCAGGGATGTGAAA | |
| | GAGTCAAGATCCAAGCAGCCACAGACTCAAAGGACATCAG | |
| | TAACTGCATGGCCAAAGCATACCCACAGTACTACAGAAAG | |
| | CCGTCAGTGGTCAAGCGGATGCCGGCCATGCTCACTGGACT | |
| | CTGTCAAGGCTGTGGCACTCGGCAGGTGGTGTTTACTAGTG | |
| | ATCCTCATAAAAGTTACCTCCCTGTGCAATTCCAGTCACCT | |
| | GATAAAGCAGAAACCCAGCGTGGAGACCCGTCTGTTATTT | |
| | CTGTCAATGGCACTGACTTTACCTTCCGAAGTGCAGGCGTC | |
| | CTCCTCCTTGTTGTGGATCCGTGCAGCGTTCCATTCCGCTTG | |
| | ACGGAAAAAACGGTTTTTCCTCTTGCTGATGTCAGTCGCAT | |
| | TGAAGAGTATTTAAAAACAGGCATCCCTCCAAGGTCCATTG | |
| | TTCTGTTGAGCACAAGAGGAGAAATAAAGCAGTTAAACAT | |
| | TTCACACTTACTAGTACCTCTGGGATTAGCCAAACCAGCTC | |
| | ATCTTTATGACAAAGGGAGTACCATATTTTTGGGATTCAGT | |
| | GGAAACTTTAAACCATCATGGACTAAGCTATTTACCAGTCC | |
| | TGCTGGACAGGGCCTTGGGGTGCTTGAACAATTCATACCTT | |
| | TGCAGCTGGACGAATATGGTTGTCCCAGAGCCACCACTGTC | |
| | CGCAGAAGAGACCTGGAACTGCTAAAGCAAGCTTCAAAAG | |
| | CACATGGTTCTAGCGAAAACCTGTATTTTCAG | |
| Nucleotide | GACGCGGCCCAGCCGGCCAGGCGCGCGCGCCGTACGAAGC | 153 |
| sequence of | TTGGTACCTCCTCAAAATATGCTCCAGATGAAAATTGCCCA | |
| Recombinant | GATCAAAATCCTCGTCTCAGGAATTGGGATCCAGGACAAG | |
| protein | ATTCTGCAAAGCAAGTTGTTATCAAGGAGGGAGATATGCT | |
| (Human | CCGTCTGACCTCAGACGCCACCGTGCATTCTATAGTCATTC | |
| TMEM2 | AGGATGGAGGACTGCTTGTATTTGGGGACAATAAAGATGG | |
| extracellular | ATCCAGAAATATTACTTTGAGGACTCATTACATCCTGATCC | |
| domain with | AGGATGGTGGGGCGCTTCATATTGGAGCAGAAAAATGCCG | |
| sequences | CTATAAATCCAAAGCGACAATTACCTTGTATGGCAAGTCAG | |
| remaining at | ATGAAGGTGAAAGTATGCCAACATTTGGCAAAAAGTTTAT | |
| C-terminus) | TGGTGTGGAAGCTGGCGGGACACTGGAGTTACATGGGGCA | |
| and without | CGGAAGGCATCGTGGACGTTGTTGGCAAGGACCCTGAATT | |
| H-tag | CCTCAGGCTTGCCCTTTGGGTCCTATACCTTTGAAAAGGAC | |
| | TTTTCCCGGGGCCTCAATGTGAGGGTCATTGACCAAGACAC | |
| | GGCCAAAATTTTGGAAAGTGAGAGATTTGATACCCATGAA | |
| | TACCGCAATGAGAGCAGGCGGCTTCAGGAGTTTCTGAGAT | |
| | TCCAGGATCCAGGTCGGATTGTTGCCATAGCTGTCGGGGAT | |
| | TCAGCCGCTAAAAGTCTCTTACAAGGAACCATCCAGATGAT | |
| | CCAGGAACGGTTGGGAAGTGAACTGATCCAAGGACTGGGC | |
| | TACAGGCAAGCTTGGGCTTTAGTTGGTGTCATTGATGGTGG | |
| | AAGCACTTCTTGCAATGAATCCGTGAGAAACTATGAAAAT | |
| | CATAGCAGTGGCGGGAAGGCTCTTGCCCAAAGAGAATTTT | |
| | ATACTGTGGATGGCCAGAAGTTCTCTGTGACAGCTTATAGT | |
| | GAATGGATTGAAGGCGTTTCTCTTTCAGGATTCCGGGTAGA | |
| | GGTTGTAGATGGAGTGAAGCTAAATTTGCTAGATGATGTTA | |
| | GTAGTTGGAAACCTGGAGACCAGATTGTGGTCGCAAGCAC | |
| | AGACTATTCCATGTACCAAGCAGAGGAGTTCACTCTTCTTC | |
| | CCTGTTCTGAATGCAGCCATTTTCAGGTCAAAGTCAAAGAA | |
| | ACCCCTCAGTTCCTGCACATGGGTGAGATCATAGACGGTGT | |
| | AGACATGAGAGCTGAGGTTGGAATTCTTACCCGGAATATT | |
| | GTGATCCAAGGAGAAGTGGAGGACTCATGCTACGCAGAAA | |
| | ATCAGTGCCAATTTTTTGATTATGATACCTTTGGGGGACAC | |
| | ATTATGATAATGAAAAATTTTACTTCAGTCCATCTTTCTTAT | |
| | GTGGAATTGAAACACATGGGTCAGCAGCAGATGGGGCGAT | |
| | ACCCTGTTCATTTTCACCTGTGTGGTGACGTGGATTATAAA | |
| | GGAGGATACAGACATGCAACATTTGTGGACGGCCTGTCTA | |
| | TTCATCACAGCTTCTCAAGGTGCATCACTGTGCATGGGACA | |
| | AATGGCTTGCTAATAAAAGACACCATTGGGTTTGACACACT | |
| | AGGTCATTGTTTCTTTTTGGAAGATGGTATTGAACAGAGGA | |
| | ATACTTTGTTCCACAATCTGGGACTCCTCACCAAGCCGGGT | |
| | ACTCTCCTGCCCACCGATAGGAACAACTCCATGTGTACCAC | |
| | CATGCGAGATAAAGTGTTTGGAAATTACATTCCTGTGCCTG | |
| | CTACTGACTGTATGGCTGTTTCAACTTTCTGGATTGCTCATC | |
| | CCAACAATAATCTGATTAATAATGCAGCTGCAGGCTCACA | |
| | GGATGCTGGAATATGGTATTTATTCCACAAGGAACCAACTG | |
| | GGGAATCCAGTGGATTGCAGCTCTTGGCAAAACCAGAACT | |
| | CACTCCATTGGGTATATTTTATAACAACAGGGTCCATTCAA | |
| | ATTTTAAGGCTGGCTTATTTATTGACAAAGGTGTCAAAACA | |
| | ACCAACTCTAGTGCTGCTGACCCAAGGGAATACCTCTGTTT | |
| | GGACAATAGTGCAAGATTTCGACCTCATCAGGATGCAAAC | |
| | CCCGAAAAACCACGTGTTGCTGCTCTAATTGACAGGCTCAT | |
| | TGCTTTTAAAAATAATGATAATGGAGCTTGGGTCAGAGGA | |
| | GGAGATATTATCGTTCAAAATTCAGCATTTGCAGATAATGG | |
| | AATAGGACTGACCTTTGCCAGTGATGGAAGCTTCCCAAGTG | |
| | ATGAAGGTTCCAGCCAAGAGGTATCTGAATCTCTCTTTGTT | |
| | GGGGAGAGCAGGAATTACGGCTTTCAGGGTGGTCAGAACA | |
| | AGTATGTAGGCACTGGAGGAATAGACCAGAAGCCTCGAAC | |
| | ATTACCCAGGAACAGGACGTTCCCAATTAGAGGCTTTCAG | |
| | ATTTATGATGGGCCCATTCATCTCACAAGGAGCACTTTCAA | |
| | AAAATATGTGCCAACTCCAGATAGGTACAGCAGTGCAATT | |
| | GGCTTCCTCATGAAGAATTCCTGGCAGATAACCCCCAGGA | |
| | ATAATATCTCCCTCGTGAAGTTTGGTCCACATGTCTCTCTG | |
| | AATGTCTTTTTTGGAAAGCCTGGTCCCTGGTTTGAAGATTG | |
| | TGAGATGGATGGTGATAAGAACTCCATATTCCATGACATTG | |
| | ATGGCTCTGTGACAGGATACAAGGATGCTTATGTGGGAAG | |
| | AATGGACAACTACCTGATCCGCCATCCAAGCTGTGTAAATG | |
| | TGTCTAAGTGGAATGCAGTGATCTGCAGTGGGACCTATGCA | |
| | CAGGTCTATGTACAGACATGGAGCACTCAGAATCTTTCTAT | |
| | GACCATTACACGAGATGAGTATCCGTCCAACCCTATGGTGC | |
| | TCCGAGGTATTAATCAGAAGGCTGCCTTTCCACAGTACCAG | |
| | CCTGTCGTCATGCTGGAGAAGGGTTATACCATCCACTGGAA | |
| | TGGGCCGGCACCACGGACTACATTTCTATACCTCGTCAACT | |
| | TCAACAAGAATGACTGGATTCGAGTTGGCCTTTGCTATCCA | |
| | TCAAACACAAGTTTTCAAGTTACCTTTGGCTATTTGCAGCG | |
| | GCAGAATGGCTCATTATCCAAAATCGAAGAATATGAGCCT | |
| | GTGCATTCACTGGAAGAACTGCAAAGAAAGCAATCCGAGA | |
| | GGAAATTCTATTTTGACTCCAGCACGGGGTTACTGTTTTTG | |
| | TATCTCAAAGCCAAAAGCCACAGGCATGGCCACAGTTACT | |
| | GTTCATCTCAGGGATGTGAAAGAGTCAAGATCCAAGCAGC | |
| | CACAGACTCAAAGGACATCAGTAACTGCATGGCCAAAGCA | |
| | TACCCACAGTACTACAGAAAGCCGTCAGTGGTCAAGCGGA | |
| | TGCCGGCCATGCTCACTGGACTCTGTCAAGGCTGTGGCACT | |
| | CGGCAGGTGGTGTTTACTAGTGATCCTCATAAAAGTTACCT | |
| | CCCTGTGCAATTCCAGTCACCTGATAAAGCAGAAACCCAG | |
| | CGTGGAGACCCGTCTGTTATTTCTGTCAATGGCACTGACTT | |
| | TACCTTCCGAAGTGCAGGCGTCCTCCTCCTTGTTGTGGATC | |
| | CGTGCAGCGTTCCATTCCGCTTGACGGAAAAAACGGTTTTT | |
| | CCTCTTGCTGATGTCAGTCGCATTGAAGAGTATTTAAAAAC | |
| | AGGCATCCCTCCAAGGTCCATTGTTCTGTTGAGCACAAGAG | |
| | GAGAAATAAAGCAGTTAAACATTTCACACTTACTAGTACCT | |
| | CTGGGATTAGCCAAACCAGCTCATCTTTATGACAAAGGGA | |
| | GTACCATATTTTTGGGATTCAGTGGAAACTTTAAACCATCA | |
| | TGGACTAAGCTATTTACCAGTCCTGCTGGACAGGGCCTTGG | |
| | GGTGCTTGAACAATTCATACCTTTGCAGCTGGACGAATATG | |
| | GTTGTCCCAGAGCCACCACTGTCCGCAGAAGAGACCTGGA | |
| | ACTGCTAAAGCAAGCTTCAAAAGCACATGGTTCTAGCGAA | |
| | AACCTGTATTTTCAG | |

### Vector

Some embodiments provide a vector comprising a nucleic acid molecule as disclosed and described herein. In some embodiments, the vector is an expression vector. In some embodiments, the vector is a eukaryotic vector. In some embodiments, the vector includes a sequence of nucleotides that directs secretion of any polypeptide encoded by a sequence of nucleotides operatively linked thereto. In some embodiments, the vector is a Pichia vector or an E. coli vector.

### Cell

Some embodiments provide a cell, comprising a vector as disclosed and described herein. In some embodiments, the cell is a prokaryotic cell. In some embodiments, the cell is a eukaryotic cell. In some embodiments, the cell is selected from among a bacterial cell, a yeast cell, a plant cell, an insect cell and an animal cell. In some embodiments, the cell is a mammalian cell.

### Methods of Preparation

Some embodiments provide a method for generating a recombinant TMEM2 protein or fragment thereof as disclosed and described herein comprising, introduction of a nucleic acid as described in SEQ ID NO: 1 optionally operably linked to a suitable promoter into a eukaryotic cell. In some embodiments, the eukaryotic cell is mammalian. In some embodiments, the eukaryotic cell is an insect. In some embodiments, the eukaryotic cell is a yeast. In some embodiments, the eukaryotic cell is a plant.

### Methods of Treatment

Some embodiments provide a method for increasing the diffusion of a therapeutic substance in a subject, comprising: administering to a subject a recombinant TMEM2 protein or fragment thereof as disclosed and described herein, whereby the diffusion of the therapeutic substance is increased. In some embodiments, the recombinant TMEM2 protein or fragment thereof as disclosed and described herein is mixed with the therapeutic substance prior to administration.

Some embodiments provide a method for increasing the diffusion of a therapeutic substance in a subject, comprising: administering to a subject a recombinant TMEM2 protein or fragment thereof as disclosed and described herein; and a therapeutic substance, whereby the diffusion of the therapeutic substance is increased. In some embodiments, the recombinant TMEM2 protein or fragment thereof as disclosed and described herein is mixed with the therapeutic substance prior to administration.

In one aspect the disclosure provides a method for dissolving hyaluronic acid fillers in a subject, comprising: administering to a subject with hyaluronic acid fillers a recombinant TMEM2 protein or fragment thereof, nucleotide, vector, cell, preparation, pharmaceutical composition, or injectable depot formulation as disclosed and described herein, whereby the dissolution of the hyaluronic acid fillers is induced. In some embodiments the disclosure provides a method for removing small areas of excess hyaluronic acid fillers in a subject, the method comprising: administering to a subject with hyaluronic acid fillers a recombinant TMEM2 protein or fragment thereof, nucleotide, vector, cell, preparation, pharmaceutical composition, or injectable depot formulation as disclosed and described herein, whereby the dissolution of the hyaluronic acid fillers is induced. In some embodiments the disclosure provides a method for complete removal of hyaluronic acid fillers, e.g., commercial HA fillers in a subject, the method comprising: administering to a subject with hyaluronic acid fillers a recombinant TMEM2 protein or fragment thereof, nucleotide, vector, cell, preparation, pharmaceutical composition, or injectable depot formulation as disclosed and described herein, whereby the dissolution of the hyaluronic acid fillers is induced. In some embodiments, the subject received an administration of excess HA fillers.

In some embodiments, the hyaluronic acid fillers are commercially available hyaluronic acid fillers. In some embodiments, the hyaluronic acid fillers comprise Juvéderm Ultra XC (Allergan Inc) or Restylane-L (Galderma Laboratories). In some embodiments, the recombinant TMEM2 protein or fragment thereof is utilized to remove small areas of excess hyaluronic acid filler. In some embodiments, the recombinant TMEM2 protein or fragment thereof is utilized to completely remove hyaluronic acid filler. In some embodiments, the subject received a hyaluronic acid filler administration at least 20 minutes, 30 minutes, 40 minutes, 50 minutes, 1 hour, 2 hours, 5 hours, 10 hours, 12 hours, 14 hours, 15 hours, 18 hours, 24 hours, 36 hours, 48 hours, 60 hours, 72 hours, 4 days, 5 days, 6 days, 1 week, 1.5 weeks, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 15 years, 20 years, 25 years, 30 years, 35 years, or 40 years before the administration of the recombinant TMEM2 protein or fragment thereof, preparation, pharmaceutical composition, or injectable depot formulation. In some embodiments, the subject received at least one, at least 2, at least 3, at least 4, at least 5, at least 6, at least 7, at least 8, at least 9, at least 10, at least 15, at least 20, at least 30, or at least 50 hyaluronic acid filler administration(s) before the administration of the recombinant TMEM2 protein or fragment thereof, preparation, pharmaceutical composition, or injectable depot formulation. In some embodiments, the subject received 11 or less, 16 or less, 22 or less, 32 or less, 52 or less, 100 or less hyaluronic acid filler administration(s) before the administration of the recombinant TMEM2 protein or fragment thereof, preparation, pharmaceutical composition, or injectable depot formulation.

Some embodiments specify methods of utilizing recombinant TMEM2 protein or fragment thereof as disclosed and described herein within clinical contexts; in some embodiments recombinant TMEM2 protein or fragment thereof as disclosed and described herein is used for non-clinical purposes.

### Methods of Administration

In any of the embodiments, administration can be by bolus injection, e.g., subcutaneous bolus injection, intramuscular bolus injection, intradermal bolus injection and the like. In any of the embodiments, administration can be by infusion, e.g., subcutaneous infusion, intramuscular infusion, intradermal infusion, and the like. In some embodiments, administration can be by direct tissue injections, including injections into tumors and masses. In some embodiments, administrations can be by subcutaneous injections.

Administration, preferably subcutaneous administration, of a pharmaceutical composition of the embodiments is accomplished using standard methods and devices, e.g., pens, injector systems, needle and syringe, a subcutaneous injection port delivery system, and the like. See, e.g., U.S. Pat. Nos. 3,547,119; 4,755,173; 4,531,937; 4,311,137; and 6,017,328, each of which is herein incorporated by reference in their entirety. A combination of a subcutaneous injection port and a device for administration of a pharmaceutical composition of the embodiments to a patient through the port is referred to herein as “a subcutaneous injection port delivery system.” In any of the embodiments, subcutaneous administration can be achieved by bolus delivery by needle and syringe.

Solutions or suspensions used for parenteral, intradermal, subcutaneous, intramuscular or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; cheating agents, such as ethylenediaminetetraacetic acid (EDTA); buffers, such as acetates, citrates and phosphates; and agents for the adjustment of tonicity, including, but not limited to sodium chloride, calcium chloride, magnesium chloride, dextrose, glycerol or boric acid. Parenteral preparations can be enclosed in ampoules, disposable syringes or single or multiple dose vials made of glass, plastic or other suitable material.

Parenteral administration of a recombinant TMEM2 protein or fragment thereof as disclosed and described herein, generally characterized by injection, either subcutaneously, intradermally, or intramuscularly is also contemplated herein. In some embodiments, parenteral administration of a recombinant TMEM2 protein or fragment thereof as disclosed and described herein includes direct tissue injections, including injections into tumors and masses. Injectables can be prepared in conventional forms, either as liquid solutions or suspensions; solid forms suitable for solution or suspension in liquid prior to injection, or as emulsions. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as, for example, sodium acetate, sorbitan monolaurate, triethanolamine oleate and cyclodextrins.

Parenteral administration of the compositions includes subcutaneous intramuscular, and intradermal administrations. In some embodiments, parenteral administration of the compositions includes direct tissue injections, including injections into tumors and masses. Preparations for parenteral administration include sterile solutions ready for injection, sterile dry soluble products, such as lyophilized powders, ready to be combined with a solvent or sterile solution just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thiomersal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the therapeutic agent and/or recombinant TMEM2 protein or fragment thereof as disclosed and described herein can be adjusted so that administration, e.g., an injection or infusion, provides an effective amount to produce the desired pharmacological effect. The exact dose depends on the age, weight and condition of the patient or animal.

The unit-dose parenteral preparations can be packaged in container, for example, an ampoule, a vial or a syringe with a needle. All preparations for parenteral administration must be sterile. In some embodiments, the unit dose can be at least 0.01 mL, at least 0.02 mL, at least 0.03 mL, at least 0.04 mL, at least 0.05 mL, at least 0.06 mL, 0.07 mL, at least 0.08 mL, at least 0.09 mL, at least 1.0 mL, at least 1.1 mL, at least 1.2 mL, at least 1.3 mL, at least 1.4 mL, at least 1.5 mL, at least 1.6 mL, at least 1.7 mL, at least 1.8 mL, at least 1.9 mL, at least 2.0 mL, at least 2.1 mL, at least 2.2 mL, at least 2.3 mL, at least 2.4 mL, at least 2.5 mL, at least 2.6 mL, at least 2.7 mL, at least 2.8 mL, at least 2.9 mL, at least 3.0 mL, at least 3.1 mL, at least 3.2 mL, at least 3.3 mL, at least 3.4 mL, or 3.5 mL. In some embodiments, the unit dose can be no more than 2.5 mL, no more than 2.4 mL, no more than 2.3 mL, no more than 2.2 mL, no more than 2.1 mL, no more than 2.0 mL, no more than 1.9 mL, no more than 1.8 mL, no more than 1.7 mL, no more than 1.6 mL, no more than 1.5 mL, no more than 1.4 mL, no more than 1.3 mL, no more than 1.2 mL, no more than 1.1 mL, no more than 1.0 mL, or less than 1.0 mL. In some embodiments, the unit-dose parenteral preparations containing a recombinant TMEM2 protein or fragment thereof as disclosed and described herein may have a volume larger than 2.5 mL.

Illustratively, subcutaneous infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.1% w/w up to about 90% w/w or more, preferably more than 1% w/w of the active compound to the treated tissue(s). The recombinant TMEM2 protein or fragment thereof as disclosed and described herein can be administered at once, or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

Formulations for injection can be presented in unit dosage form, e.g., in ampoules or in multi-dose containers, optionally with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use. For example, provided herein are parenteral formulations containing an effective amount of hyaluronidase, such as 0.5 to 500,000 Units, in a stabilized solution or a lyophilized from.

Some embodiments also include pharmaceutical formulations for parenteral administration, e.g., by bolus injection or continuous infusion, include aqueous suspensions of the synthetic membrane vesicles, having encapsulated therein a therapeutic agent. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. In some embodiments the formulation can be administered by subcutaneous injection.

The effective daily dosage of the therapeutic agent may be varied over a wide range; e.g., from about 0.1 μg to about 10,000 mg per adult human per day.

Therapeutically effective dosages of a therapeutic agent can range from 0.1 μg to about 0.5 μg per dose, from about 0.5 μg to about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, from about 35 mg to about 40 mg per dose, from about 40 mg to about 50 mg per dose, from about 50 mg to about 75 mg per dose, from about 75 mg to about 100 mg per dose, from about 100 mg to about 200 mg per dose, from about 200 mg to about 400 mg per dose, from about 400 mg to about 600 mg per dose, from about 600 mg to about 800 mg per dose, or from about 800 mg to about 5000 mg per dose. An effective amount of the instant compounds is ordinarily supplied at a dosage level of from about 0.01 mg/kg to about 150 mg/kg of body weight per day. Typically, the range is from about 0.1 to about 80 mg/kg of body weight per day, and especially from about 0.2 mg/kg to about 40 mg/kg of body weight per day. The compounds may be administered on a regimen of about 1 to about 10 times per day, or continuous infusion for a period of from about 1, 5, 10, 15, 30 or 60 minutes, or 1, 2, 3, 5, 7, 10, 12, 15, 18, 20 or 24 hours or a range defined by any two of the preceding values.

Effective dosages of recombinant TMEM2 protein or fragment thereof as disclosed and described herein can range from 0.1 United States Pharmacopeia (USP) units to about 0.5 USP units per dose, from about 0.5 USP units to about 1.0 USP units per dose, from about 1.0 USP units per dose to about 5.0 USP units per dose, from about 5.0 USP units to about 10 USP units per dose, from about 10 USP units to about 20 USP units per dose, from about 20 USP units per dose to about 30 USP units per dose, from about 30 USP units per dose to about 40 USP units per dose, from about 40 USP units per dose to about 50 USP units per dose, from about 50 USP units per dose to about 60 USP units per dose, from about 60 USP units per dose to about 70 USP units per dose, from about 70 USP units to about 80 USP units per dose, from about 80 USP units per dose to about 100 USP units per dose, from about 100 USP units to about 150 USP units per dose, from about 150 USP units to about 200 USP units per dose, from about 200 USP units per dose to about 250 USP units per dose, from about 250 USP units to about 300 USP units per dose, from about 300 USP units to about 450 USP units per dose, from about 450 USP units to about 600 USP units per dose, from about 600 USP units to about 750 USP units per dose, from about 750 USP units to about 900 USP units per dose, from about 900 USP units to about 1050 USP units per dose, from about 1050 USP units to about 1200 USP units per dose, from about 1200 USP units to about 1350 USP units per dose, or from about 1350 USP units to about 1500 USP units per dose.

Some embodiments provide a method of administering recombinant TMEM2 protein or fragment thereof as disclosed and described herein to a subject, once about every 5, 10, 12, 15, 20 or 24 hrs. In some embodiments, the recombinant TMEM2 protein or fragment thereof as disclosed and described herein is administered to a subject at least 1, 2, 3, 4, 5, or 6 times daily.

In some embodiments, 2, 3, 4, 5, 6, or more doses of a therapeutic agent are administered to a subject following an administration of recombinant TMEM2 protein or fragment thereof as disclosed and described herein, where each of the doses is separated by a period of time. In some embodiments the period of time is at least, or is at least about, 1 minute, 5 minutes, 10 minutes, 15 minutes, 30 or minutes. In other embodiments, the period of time is at least, or is at least about, 1, 2, 3, 4, 5, 6, 10, 12, or 15 hours. In some embodiments, the period of time is not more than about 20 hours. In some embodiments, the period is a range defined by any of the preceding values, e.g., 15 minutes to 2 hours.

### Kit

Some embodiments provide a kit, comprising:
(a) a recombinant TMEM2 protein or fragment thereof as disclosed and described herein at a dose between 0.1 and 1500 Units/mL in an acceptable carrier;
(b) at least one therapeutic substance in a pharmaceutically acceptable carrier; and
(c) optionally, instructions for delivering therapeutic substances. In some embodiments, the recombinant TMEM2 protein or fragment thereof as disclosed and described herein and the therapeutic substance are provided as a mixture. In some embodiments, the kit comprises a first sterile container comprising a therapeutic agent, and a second sterile container comprising the recombinant TMEM2 protein or fragment thereof as disclosed and described herein. In some embodiments, the first and second containers are chambers of a two-chamber syringe.

### Depot Formulation

Some embodiments provide an injectable depot formulation comprising i) a recombinant TMEM2 protein or fragment thereof as disclosed and described herein; and ii) a therapeutic substance. In some embodiments, the therapeutic substance is in the form of particles having an average particle size of less than about 60000 nm, 50000 nm, 40000 nm, 20000 nm, 10000 nm, 2000 nm, 1500 nm, 1000 nm, 500 nm, 250 nm, 100 nm or 50 nm, or an average particle size within a range defined by any of the preceding values. In some embodiments, the particles are resistant to deformation or amenable to deformation at physiological temperatures.

### Therapeutic Agents

In some embodiments, the pharmaceutical composition or a recombinant protein described herein is administered in combination with one or more additional therapeutic agents. In some embodiments, the additional therapeutic agents are co-formulated and administered as a single pharmaceutical composition. In some embodiments, the additional therapeutic agents are not co-formulated and administered as additional pharmaceutical compositions.

In some embodiments, the pharmaceutical composition or a recombinant protein described herein increases the dispersion and absorption of co-administered therapeutics.

A non-limiting list of therapeutic agent classes include, but are not limited to, antianginas, antiarrhythmics, antiasthmatic agents, antibiotics, antidiabetics, antifungals, antihistamines, antihypertensives, antiparasitics, antineoplastics, antiviral agents, otologicals, cardiac glycosides, hormones, immunomodulators, monoclonal antibodies, neurotransmitters, sedatives, vaccines, vasopressors, anesthetics, amide anaesthetics, corticosteroids, tricyclic antidepressants, tetracyclic antidepressants, selective serotonin reuptake inhibitors, steroid receptor modulators, antipsychotic drugs, antiprotozoal drugs, opioids, antiproliferative agents, salicylanilides, antihelminthic drugs, vinca alkaloids, anti-inflammatory agents, antidepressants, prostaglandins, phosphodiesterase IV inhibitors; retinoids, steroids, β-adrenergic receptor ligands, anti-mitotic agents, microtubule inhibitors, microtubule-stabilizing agents, serotonin norepinephrine reuptake inhibitors, noradrenaline reuptake inhibitors, non-steroidal immunophilin-dependent immunosuppressants, non-steroidal immunophilin-dependent immunosuppressant enhancers; antimalarial agents, analgesics, immunosuppressants, expectorants, sulfa drugs, cardiovascular drugs, central nervous system (CNS) depressants, H2-blockers, anti-platelet drugs, anticonvulsants, alpha blockers, beta-blockers, cholinesterase inhibitors, calcium channel blockers, H1-receptor antagonists, and proteinaceous materials. The therapeutic agents listed herein can be used in the preparation of medicaments for the treatment of a disease for which the therapeutic agent is known to those of skill in the art to be effective. Therapeutic agents, and diseases for which the therapeutic agent is effective, can be identified by reference to, for example, The Physicians' Desk Reference, 71st edition, 2016, PDR Staff, PDR Network, NJ, United States or The Prescribers' Digital Reference (PDR), each of which is incorporated herein by reference in its entirety. In some embodiments, the therapeutic agent is an agent listed in U.S. Pat. No. 10,588,983 B2 of which is incorporated herein by reference in its entirety. In some embodiments, the therapeutic agent is a monoclonal antibody, or fragment thereof. In some embodiments, the therapeutic agent is a broadly neutralizing antibody, or fragment thereof. In some embodiments, the monoclonal antibody is daratumumab, trastuzumab, trastuzumab, pertuzumab, or rituximab. In some embodiments, the therapeutic agent is efgartigimod alfa, nivolumab, atezolizumab, ARGX-117, relatlimab, Anti-TIM 3, CAP256V2LS, Teprotumumab-TRBW, ocrelizumab, TAK-881, cabotegravir, or N6LS bNAb.

Examples of monoclonal antibodies, include but are not limited to, abagovomab, abatacept, abciximab, adalimumab, adecatumumab, aflibercept, afutuzumab, alacizumab pegol, alemtuzumab, altumomab, afelimomab, anatumomab mafenatox, anrukinzumab, apolizumab, arcitumomab, aselizumab, atlizumab, atorolimumab, bapineuzumab, basiliximab, bavituximab, bectumomab, belatacept, belimumab, bertilimumab, besilesomab, bevacizumab, biciromab brallobarbital, bivatuzumab mertansine, blinatumomab, briakinumab, canakinumab, cantuzumab mertansine, capromab pendetide, catumaxomab, cedelizumab, certolizumab pegol, cetuximab, citatuzumab bogatox, cixutumumab, clenoliximab, golimumab, ustekinumab, conatumumab, dacetuzumab, dacliximab, daclizumab, denosumab, detumomab, dorlimomab aritox, dorlixizumab, ecromeximab, eculizumab, edobacomab, edrecolomab, efalizumab, efungumab, elsilimomab, enlimomab pegol, epitumomab cituxetan, epratuzumab, erlizumab, ertumaxomab, etanercept, etaracizumab, exbivirumab, fanolesomab, faralimomab, felvizumab, fezakinumab, figitumumab, fontolizumab, foravirumab, galiximab, gantenerumab, gavilimomab, gemtuzumab ozogamicin, golimumab, gomiliximab, ibalizumab, ibritumomab tiuxetan, igovomab, imciromab, infliximab, intetumumab, inolimomab, inotuzumab ozogamicin, ibalizumab, ipilimumab, iratumumab, keliximab, labetuzumab, lemalesomab, lebrilizumab, lerdelimumab, lexatumumab, libivirumab, lintuzumab, lucatumumab, lumiliximab, mapatumumab, maslimomab, matuzumab, mepolizumab, metelimumab, milatuzumab, minretumomab, mitumomab, morolimumab, motavizumab, muromonab, stamulumab, nacolomab tafenatox, naptumomab estafenatox, natalizumab, nebacumab, necitumumab, nerelimomab, nimotuzumab, nofetumomab merpentan, ocrelizumab, odulimomab, ofatumumab, omalizumab, oportuzumab monatox, oregovomab, otelixizumab, pagibaximab, palivizumab, panitumumab, panobacumab, pascolizumab, pemtumomab, pertuzumab, pexelizumab, pintumomab, priliximab, pritumumab, rafivirumab, ramucirumab, ranibizumab, raxibacumab, regavirumab, reslizumab, rilonacept, rilotumumab, rituximab, robatumumab, rovelizumab, rozrolimupab, ruplizumab, satumomab, sevirumab, sibrotuzumab, siltuximab, siplizumab, solanezumab, sonepcizumab, sontuzumab, stamulumab, sulesomab, tacatuzumab tetraxetan, tadocizumab, talizumab, tanezumab, taplitumomab paptox, tefibazumab, telimomab aritox, tenatumomab, teneliximab, teplizumab, ticilimumab, tigatuzumab, tocilizumab, toralizumab, tositumomab, trastuzumab, tremelimumab, tucotuzumab celmoleukin, tuvirumab, urtoxazumab, ustekinumab, vapaliximab, vedolizumab, veltuzumab, vepalimomab, visilizumab, volociximab, votumumab, zalutumumab, zanolimumab, ziralimumab, and zolimomab aritox.

In some aspects, in the methods disclosed herein, the pharmaceutical composition disclosed herein is administered to a subject via a route selected from the group consisting of subcutaneous injection, intramuscular injection, and intravenous injection. In some embodiments, in the methods disclosed herein, the pharmaceutical composition disclosed herein is administered to a subject via subcutaneous injection. In some embodiments, in the methods disclosed herein, the pharmaceutical composition disclosed herein is administered to a subject via intramuscular injection. In some embodiments, in the methods disclosed herein, the pharmaceutical composition disclosed herein is administered to a subject via intravenous injection.

In some aspects, in the methods disclosed herein, the pharmaceutical composition disclosed herein further comprises a delivery vehicle selected from the group consisting of liposomes, nanoparticles, microparticles, microspheres, lipid particles, vesicles, poloxamers, and polycationic materials. In some embodiments, in the methods disclosed herein, the pharmaceutical composition disclosed herein further comprises liposomes as a delivery vehicle. In some embodiments, in the methods disclosed herein, the pharmaceutical composition disclosed herein further comprises nanoparticles as a delivery vehicle. In some embodiments, in the methods disclosed herein, the pharmaceutical composition disclosed herein further comprises microparticles as a delivery vehicle. In some embodiments, in the methods disclosed herein, the pharmaceutical composition disclosed herein further comprises microspheres as a delivery vehicle. In some embodiments, in the methods disclosed herein, the pharmaceutical composition disclosed herein further comprises lipid particles as a delivery vehicle. In some embodiments, in the methods disclosed herein, the pharmaceutical composition disclosed herein further comprises vesicles as a delivery vehicle. In some embodiments, in the methods disclosed herein, the pharmaceutical composition disclosed herein further comprises poloxamers as a delivery vehicle. In some embodiments, in the methods disclosed herein, the pharmaceutical composition disclosed herein further comprises polycationic materials as a delivery vehicle.

### Definitions

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the singular forms “a,” “an,” and “the” are intended to include the plural forms as well, unless the context clearly indicates otherwise. For example, the term “a sample” includes a plurality of samples, including mixtures thereof. Furthermore, to the extent that the terms “including,” “includes,” “having,” “has,” “with,” or variants thereof are used in either the detailed description and/or the claims, such terms are intended to be inclusive in a manner similar to the term “comprising.”

The terms “measuring” and “assaying” are often used interchangeably herein to refer to forms of measurement. The terms include determining if an element is present or not (for example, detection). These terms can include quantitative, qualitative, or quantitative and qualitative determinations.

The section headings used herein are for organizational purposes only and are not to be construed as limiting the subject matter described.

Any methods described herein are modular. Accordingly, terms such as “first” and “second” do not necessarily imply priority, order of importance, or order of acts.

Use of absolute or sequential terms, for example, “first,” “initially,” “subsequently,” “before,” “after,” and “finally,” are not meant to limit scope of the present embodiments disclosed herein but as exemplary.

As used herein, the phrases “at least one,” “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject is not necessarily diagnosed or suspected of being at high risk for the disease.

As used herein, the term “in vivo” can be used to describe an event that takes place in an organism, such as a subject's body.

As used herein, the term “ex vivo” can be used to describe an event that takes place outside of an organism such as subject's body. An “ex vivo” assay cannot be performed on a subject. Rather, it can be performed upon a sample separate from a subject. Ex vivo can be used to describe an event occurring in an intact cell outside a subject's body.

As used herein, the term “in vitro” can be used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject can still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

The terms “increased,” “increasing,” or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased,” “decreasing,” or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level.

The terms “individual” or “subject” are used interchangeably and encompass mammals. Non-limiting examples of mammal include any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents such as rats, mice and guinea pigs, and the like. The mammal can be a human. The term “animal” as used herein comprises human beings and non-human animals. In one embodiment, a “non-human animal” is a mammal, for example a rodent such as rat or a mouse. A “patient,” as used herein refers to a subject that has, or has been diagnosed with, a disease or a condition described herein.

“Treat, “treating,” or “treatment,” as used herein, refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

“Treatment” of an individual (e.g., a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

An “effective amount” or “therapeutically effective amount” refers to an amount of a compound administered to a mammalian subject, either as a single dose or as part of a series of doses, which is effective to produce a desired therapeutic effect.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio.

As used herein, the term “administration,” “administering” and variants thereof means introducing a composition or agent into a subject and includes concurrent and sequential introduction of a composition or agent.

### EXAMPLES

The following illustrative examples are representative of embodiments of the stimulation, systems, and methods described herein and are not meant to be limiting in any way.

### Materials and Methods

### Example 1—Design of the Expression Vector

Synthetic cDNAs (produced by Genscript) for extracellular domain of mouse (94-1383aa) and human (104-1383aa) TMEM2 were cloned into pSecTag2 (Invitrogen) vector with Ig signal peptide at N-terminus and 6His-tag at C-terminus. The correct sequence was verified by sequencing. The vector encodes a protein consisting of the N-terminal signal peptide, a C-terminal hexa-histidine tag, a TEV protease cleavage site, and TMEM2 residues 104-1383 (SSKYAPDENC . . . ELLKQASKAH; SEQ ID NO. 1). After cleavage of the signal peptide, the following vector-derived sequence remains at the N-terminus of human TMEM2 extracellular domain: DAAQPARRARRTKLGT (SEQ ID NO. 8). Also, either of the following sequence remains at the C-terminus of human TMEM2 extracellular domain: GSSENLYFQGSSHHHHHH (SEQ ID NO. 5) or GSSENLYFQGSS (SEQ ID NO. 6).

### Example 2—Recombinant TMEM2 Production

Freestyle 293 cells (Invitrogen) were grown to the final volume in serum-free Freestyle 293 medium and were transfected with maxiprep endotoxin-free DNA using 293fectin. Transient expression was carried out for 72 hours in shaker culture, 37° C., 8% CO₂. After cell removal by centrifugation secreted TMEM2 was purified from conditioned media using Co-NTA affinity chromatography, followed by size exclusion FPLC on Superdex 200 column. SEC fraction containing TMEM2 were pooled, dialyzed into 1×PBS, concentrate, sterile-filtered and flash frozen in liquid nitrogen. Typical yield was ˜10 mg from 1 L of culture with >95% purity as assessed by Coomassie stained SDS PAGE. Table 1 provides the sequences of all the Human TMEM2 Extracellular Domain fragments cloned.

TABLE 1
Human TMEM2 Extracellular Domain (104-1383 aa)

| AA | Number | SEQ ID NO: |
|---|---|---|
| SSKYAPDENC | 1-10 | 20 |
| PDQNPRLRNW | 11-20 | 21 |
| DPGQDSAKQV | 21-30 | 22 |
| VIKEGDMLRL | 31-40 | 23 |
| TSDATVHSIV | 41-50 | 24 |
| IQDGGLLVFG | 51-60 | 25 |
| DNKDGSRNIT | 61-70 | 26 |
| LRTHYILIQD | 71-80 | 27 |
| GGALHIGAEK | 81-90 | 28 |
| CRYKSKATIT | 91-100 | 29 |
| LYGKSDEGES | 101-110 | 30 |
| MPTFGKKFIG | 111-120 | 31 |
| VEAGGTLELH | 121-130 | 32 |
| GARKASWTLL | 131-140 | 33 |
| ARTLNSSGLP | 141-150 | 34 |
| FGSYTFEKDF | 151-160 | 35 |
| SRGLNVRVID | 161-170 | 36 |
| QDTAKILESE | 171-180 | 37 |
| RFDTHEYRNE | 181-190 | 38 |
| SRRLQEFLRF | 191-200 | 39 |
| QDPGRIVAIA | 201-210 | 40 |
| VGDSAAKSLL | 211-220 | 41 |
| QGTIQMIQER | 221-230 | 42 |
| LGSELIQGLG | 231-240 | 43 |
| YRQAWALVGV | 241-250 | 44 |
| IDGGSTSCNE | 251-260 | 45 |
| SVRNYENHSS | 261-270 | 46 |
| GGKALAQREF | 271-280 | 47 |
| YTVDGQKFSV | 281-290 | 48 |
| TAYSEWIEGV | 291-300 | 49 |
| SLSGFRVEVV | 301-310 | 50 |
| DGVKLNLLDD | 311-320 | 51 |
| VSSWKPGDQI | 321-330 | 52 |
| VVASTDYSMY | 331-340 | 53 |
| QAEEFTLLPC | 341-350 | 54 |
| SECSHFQVKV | 351-360 | 55 |
| KETPQFLHMG | 361-370 | 56 |
| EIIDGVDMRA | 371-380 | 57 |
| EVGILTRNIV | 381-390 | 58 |
| IQGEVEDSCY | 391-400 | 59 |
| AENQCQFFDY | 401-410 | 60 |
| DTFGGHIMIM | 411-420 | 61 |
| KNFTSVHLSY | 421-430 | 62 |
| VELKHMGQQQ | 431-440 | 63 |
| MGRYPVHFHL | 441-450 | 64 |
| CGDVDYKGGY | 451-460 | 65 |
| RHATFVDGLS | 461-470 | 66 |
| IHHSFSRCIT | 471-480 | 67 |
| VHGTNGLLIK | 481-490 | 68 |
| DTIGFDTLGH | 491-500 | 69 |
| CFFLEDGIEQ | 501-510 | 70 |
| RNTLFHNLGL | 511-520 | 71 |
| LTKPGTLLPT | 521-530 | 72 |
| DRNNSMCTTM | 531-540 | 73 |
| RDKVFGNYIP | 541-550 | 74 |
| VPATDCMAVS | 551-560 | 75 |
| TFWIAHPNNN | 561-570 | 76 |
| LINNAAAGSQ | 571-580 | 77 |
| DAGIWYLFHK | 581-590 | 78 |
| EPTGESSGLQ | 591-600 | 79 |
| LLAKPELTPL | 601-610 | 80 |
| GIFYNNRVHS | 611-620 | 81 |
| NFKAGLFIDK | 621-630 | 82 |
| GVKTTNSSAA | 631-640 | 83 |
| DPREYLCLDN | 641-650 | 84 |
| SARFRPHQDA | 651-660 | 85 |
| NPEKPRVAAL | 661-670 | 86 |
| IDRLIAFKNN | 671-680 | 87 |
| DNGAWVRGGD | 681-690 | 88 |
| IIVQNSAFAD | 691-700 | 89 |
| NGIGLTFASD | 701-710 | 90 |
| GSFPSDEGSS | 711-720 | 91 |
| QEVSESLFVG | 721-730 | 92 |
| ESRNYGFQGG | 731-740 | 93 |
| QNKYVGTGGI | 741-750 | 94 |
| DQKPRTLPRN | 751-760 | 95 |
| RTFPIRGFQI | 761-770 | 96 |
| YDGPIHLTRS | 771-780 | 97 |
| TFKKYVPTPD | 781-790 | 98 |
| RYSSAIGFLM | 791-800 | 99 |
| KNSWQITPRN | 801-810 | 100 |
| NISLVKFGPH | 811-820 | 101 |
| VSLNVFFGKP | 821-830 | 102 |
| GPWFEDCEMD | 831-840 | 103 |
| GDKNSIFHDI | 841-850 | 104 |
| DGSVTGYKDA | 851-860 | 105 |
| YVGRMDNYLI | 861-870 | 106 |
| RHPSCVNVSK | 871-880 | 107 |
| WNAVICSGTY | 881-890 | 108 |
| AQVYVQTWST | 891-900 | 109 |
| QNLSMTITRD | 901-910 | 110 |
| EYPSNPMVLR | 911-920 | 111 |
| GINQKAAFPQ | 921-930 | 112 |
| YQPVVMLEKG | 931-940 | 113 |
| YTIHWNGPAP | 941-950 | 114 |
| RTTFLYLVNF | 951-960 | 115 |
| NKNDWIRVGL | 961-970 | 116 |
| CYPSNTSFQV | 971-980 | 117 |
| TFGYLQRQNG | 981-990 | 118 |
| SLSKIEEYEP | 991-1000 | 119 |
| VHSLEELQRK | 1001-1010 | 120 |
| QSERKFYFDS | 1011-1020 | 121 |
| STGLLFLYLK | 1021-1030 | 122 |
| AKSHRHGHSY | 1031-1040 | 123 |
| CSSQGCERVK | 1041-1050 | 124 |
| IQAATDSKDI | 1051-1060 | 125 |
| SNCMAKAYPQ | 1061-1070 | 126 |
| YYRKPSVVKR | 1071-1080 | 127 |
| MPAMLTGLCQ | 1081-1090 | 128 |
| GCGTRQVVFT | 1091-1100 | 129 |
| SDPHKSYLPV | 1101-1110 | 130 |
| QFQSPDKAET | 1111-1120 | 131 |
| QRGDPSVISV | 1121-1130 | 132 |
| NGTDFTFRSA | 1131-1140 | 133 |
| GVLLLVVDPC | 1141-1150 | 134 |
| SVPFRLTEKT | 1151-1160 | 135 |
| VFPLADVSRI | 1161-1170 | 136 |
| EEYLKTGIPP | 1171-1180 | 137 |
| RSIVLLSTRG | 1181-1190 | 138 |
| EIKQLNISHL | 1191-1200 | 139 |
| LVPLGLAKPA | 1201-1210 | 140 |
| HLYDKGSTIF | 1211-1220 | 141 |
| LGFSGNFKPS | 1221-1230 | 142 |
| WTKLFTSPAG | 1231-1240 | 143 |
| QGLGVLEQFI | 1241-1250 | 144 |
| PLQLDEYGCP | 1251-1260 | 145 |
| RATTVRRRDL | 1261-1270 | 146 |
| ELLKQASKAH | 1271-1280 | 147 |

### Example 3—Determination of TMEM2 Ability as a Spreading Agent

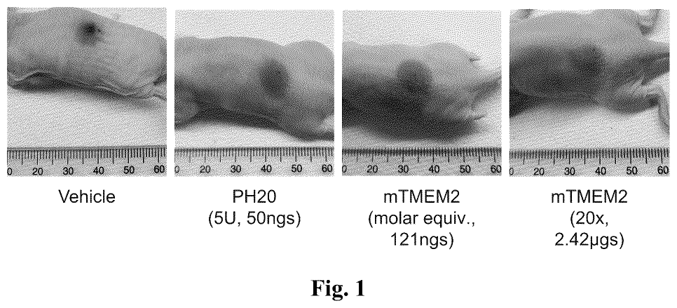

Mouse TMEM2 extracellular domain was used as a spreading agent in a mouse model in comparison to recombinant PH-20 Protein used as a comparator. It was seen in FIG. 1 that Mouse TMEM2 extracellular domain affords similar spreading effect as recombinant PH-20 Protein at a 20 min. time point.

### Example 4—Similarity of hTMEM2 Models Generated by Cryo-EM and Alphafold2

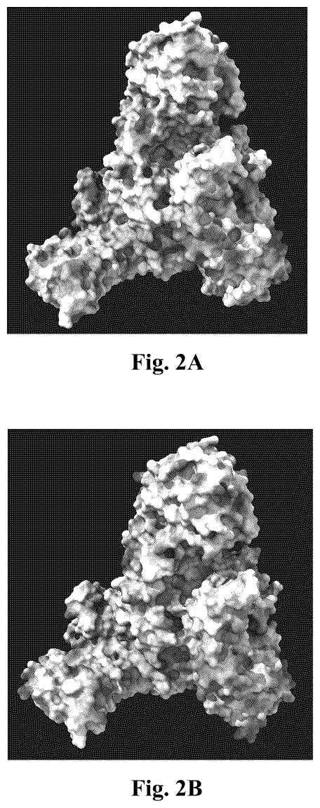

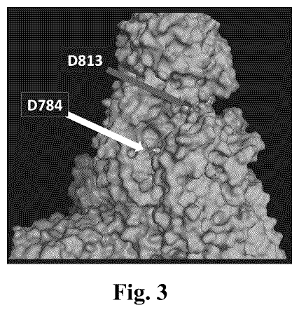

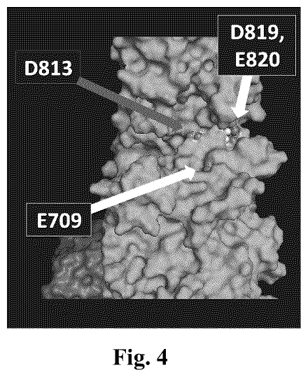

Models of hTMEM2 were generated using both Alphafold2 and Cryo-EM. As shown in FIG. 2A and FIG. 2B the models of hTMEM2 elucidated by Cryo-EM (FIG. 2A) and predicted by Alphafold2 (FIG. 2B) were highly similar. Additionally, as shown in FIG. 3 and FIG. 4 acidic amino acid residues are identified on the surface in the Cryo-EM model.

### Example 5—Docking Experiments for hTMEM2

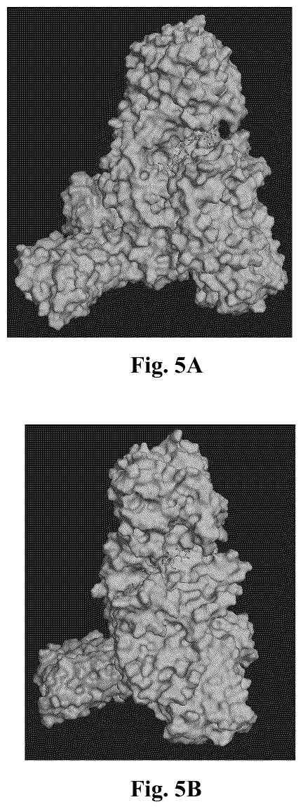

Docking experiments were performed to assess hTMEM2 and hyaluronan interactions. A computational model of 8-mer hyaluronan determined by NMR and computer modeling (PBD ID: 2BVK) was modified in ChemDoodle 3D to 6-mer hyaluronan and then further processed in Dock Prep wherein the solvent was deleted, hydrogen atoms were added, and charges assigned (Gasteiger). Additionally, a computational model of hTMEM2 (with Ca⁺) determined by cryo-EM was processed in Dock Prep wherein the solvent was deleted, hydrogen atoms were added, and charges assigned (Gasteiger). AutoDock Vina was used to assess the docking compatibility of the Dock Prep-modified computational models of hexamer hyaluronan and hTMEM2. The docking location of the number one score candidate out of the ten candidates is shown in FIG. 5A and a ninety-degree rotated version of the docking location of the number one score candidate out of the ten candidates is shown in FIG. 5B.

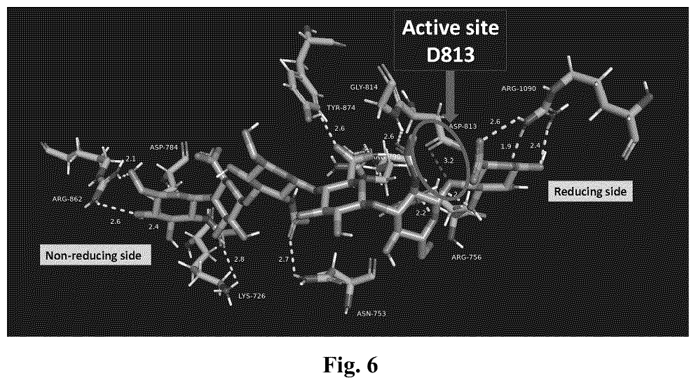

The interaction between hTMEM2 and hyaluronan was further assessed and as the interaction between the hTMEM2 and hyaluronan were shown in FIG. 6 and Table 4.

TABLE 4
Interaction between the hTMEM2 and hyaluronan

| HA | HA | hTMEM2 | hTMEM2 | |
|---|---|---|---|---|
| Sugar | Atom | Residues | Atoms | Distance (Å) |
| −5GlcNAc | O7 | K726 | HZ2 | 2.8 |
| −5GlcNAc | HO4 | D784 | OD2 | 2.4 |
| −5GlcNAc | O4 | R862 | HH22 | 2.6 |
| −5GlcNAc | O6 | R862 | HH12 | 2.1 |
| −4GlcA | O7 | N753 | HD21 | 2.7 |
| −2GlcA | O6B | Y874 | HH | 2.6 |
| −2GlcA | O6A | R790 | HH12 | 2.3 |
| −1GlcNAc | O7 | G814 | H | 2.6 |
| −1GlcNAc | O5 | R756 | HH11 | 2.2 |
| +1GlcA | O4 (Glycosidic bond) | D813 | OD1 | 3.2 |
| +1GlcA | O3 | R756 | HH12 | 2.7 |
| +1GlcA | O6B | R1090 | HH21 | 2.6 |
| +1GlcA | O1 | R1090 | HH12 | 2.4 |
| +1GlcA | O5 | R1090 | HH22 | 1.9 |

### Example 6—Hyaluronidase Assay with P2 Membrane Fraction

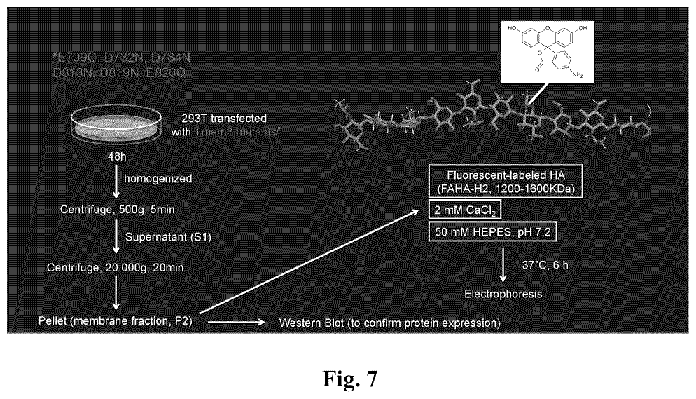

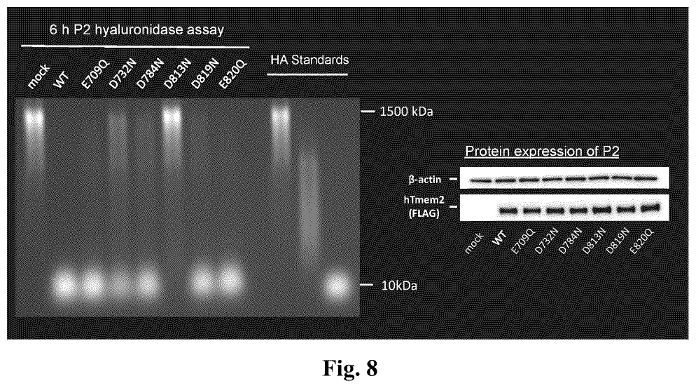

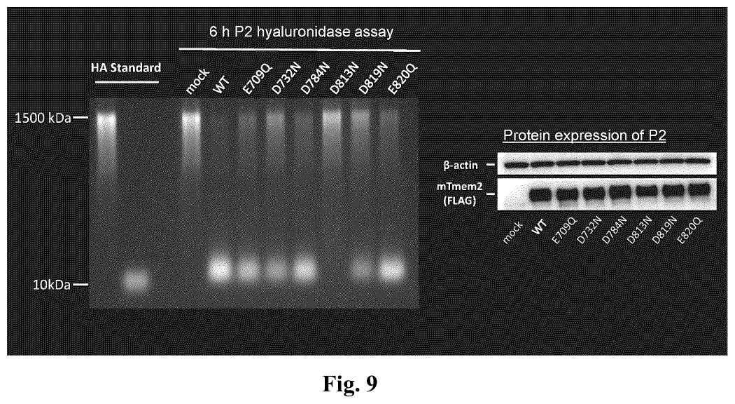

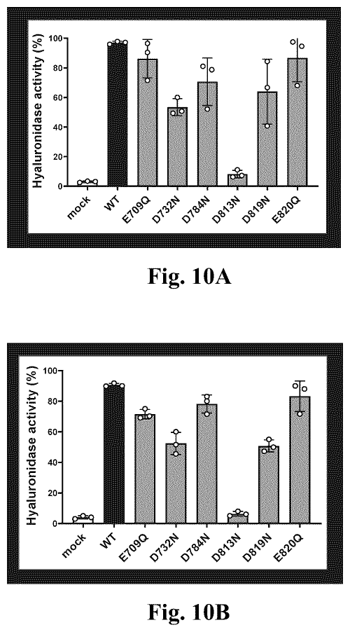

Hyaluronidase assay experiments were performed with P2 membrane fractions using different human and mouse TMEM2 membrane mutants. 293T cells were transfected with human and mouse wild type TMEM2, E709Q, D732N, D784N, D813N, D819N, and E820Q mutants. 293T cells transfected with mock constructs were used as control. The cells were allowed to grow for 48 hours. At the end of 48 hours, the cells were homogenized followed by centrifugation at 500 g for 5 minutes. The supernatant (Si) was collected and further subjected to centrifugation at 20,000 g for 20 mins. The pellet containing the membrane fraction P2 were collected. Western Blot was performed using a portion of the pellets collected to confirm protein expression. The remaining portion of the membrane fraction P2 were flagged with Fluorescent-labeled HA (FAHA-H2, 1200-1600 KDa) and were incubated in presence of 2 mM CaCl₂ and 50 mM HEPES (pH 7.2) at 37° C. for 6 hours. At the end of 6 hours the samples were collected and were run in an electrophoresis gel. FIG. 7 provides a flow chart description of the experiment conducted. FIG. 8 left panel shows results from the 6h P2 hyaluronidase assay using human wild type TMEM2 and various TMEM2 mutants and FIG. 8 right panel shows the protein expression results from the western blot ran with wild type hTMEM2 and hTMEM2 mutants. FIG. 9 left panel shows results from the 6h P2 hyaluronidase assay using mouse wild type TMEM2 and various TMEM2 mutants and FIG. 9 right panel shows the protein expression results from the western blot ran with wild type mTMEM2 and hTMEM2 mutants. The percentage of hyaluronidase activity for wildtype TMEM2 were quantified and were shown in FIG. 10A (human) and 10B (mouse).

### Example 7—Hyaluronidase Assay with Purified TMEM2-Extracellular Domain (ECD)

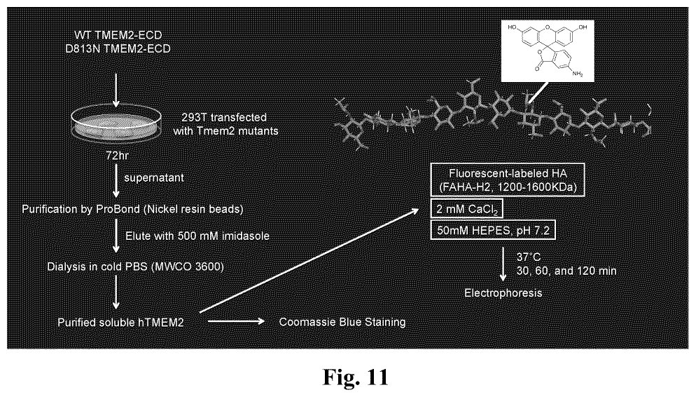

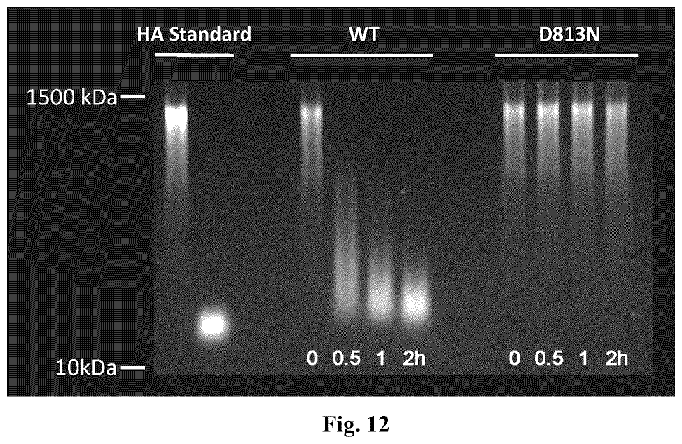

The effectiveness of TMEM2-extracellular domain as a hyaluronidase was investigated using hyaluronidase activity assay experiments. 293T cells were transfected with wildtype and D813N mutant TMEM2-extracellular domain. 293T cells transfected with mock constructs were used as control. The cells were allowed to grow for 72 hours. At the end of 72 hours, the cells were homogenized followed by centrifugation. The supernatant was purified with ProBond (nickel resin beads), eluted with 500 mM imidazole, then exposed to dialysis in cold PBS (Molecular weight cut-off 3600 Da). Coomassie blue staining was performed using a portion of the purified soluble hTMEM2 to confirm protein expression. The remaining portion of the purified soluble hTMEM2 was flagged with fluorescent-labeled HA (FAHA-H2, 1200-1600 KDa) and was incubated in the presence of 2 mM CaCl₂ and 50 mM HEPES (pH 7.2) at 37° C. for 30, 60, and 120 minutes. At the end of the time course the samples were collected and were run in an electrophoresis gel. A flowchart overview of the hyaluronidase assay experiments incorporating purified TMEM2-extracellular domain (ECD) is shown in FIG. 11. The results of the hyaluronidase assay experiments for purified wildtype and D813N mutant TMEM2-ECD incubated with fluorescent-labeled HA for 0.5, 1, and 2 hours is shown in FIG. 12.

### Example 8—D813N Mutation Effects on TMEM2-Dependent Cellular Function

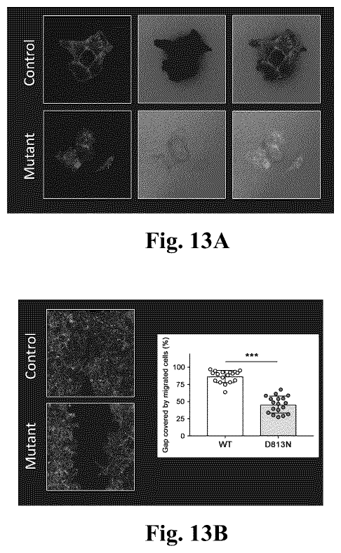

The effect of a D813N mutation on cellular function was assessed. 293T cells were transfected with wildtype and D813N mutant TMEM2. The effect of wildtype and mutant TMEM2 transfection on in situ degradation of HA is shown in FIG. 13A. The effect of wildtype and mutant TMEM2 transfection on cell migration of HA is shown in FIG. 13B.

### Example 9—Effectiveness of Recombinant Protein to Remove Hyaluronic Acid Filler Nodules

The effectiveness of the recombinant TMEM2 protein at removing HA filler nodules is assessed. Human subjects participate in a split-arm, parallel-group, randomized clinical trial with a 1:1 allocation ratio and a block size of 2 for allocation to HA filler type. There is 1:1:1:1 allocation to 1 of 3 microconcentrations of hyaluronidase (plus 1 control arm), with a block size of 4. The unit of randomization is the individual unilateral button of hyaluronidase. Each subject has 8 treatment sites along their bilateral upper inner (medial) arms, 4 on each arm, with sites 5 cm apart and marked with ink spots from a surgical pen. For each participant, each arm receives either of 2 types of HA filler (Juvéderm Ultra XC, Allergan Inc; or Restylane-L, Galderma Laboratories). Each arm is injected with 4 separate aliquots of the assigned HA filler (volume of 0.4 mL each) placed 1.5 cm medial to each of the 4 marked sites. Each aliquot is approximately round and button-shaped and placed at the dermal-subcutaneous junction and is delivered from a single point of insertion using a standard syringe apparatus with a fresh 30-gauge needle. At one, two, and three weeks after aliquot administration subjects are treated with 0.1 mL of varying doses of recombinant TMEM2 protein (1.5, 3.0, or 9.0 U per 0.1 mL) or saline control at each of the four HA filler aliquot sites on each arm. Exactly 1 button on each arm receives 0.1 mL of each of the 4 investigational products: (1) 1.5 U/0.1 mL recombinant TMEM2 protein, (2) 3.0 U/0.1 mL, (3) 9.0 U/0.1 mL, or (4) 0.1 mL normal saline control. Once a button is assigned to a particular treatment arm, the button is injected with the same concentration of investigational products at each treatment visit 1, 2, and 3 weeks after HA filler placement. The different concentrations are achieved using normal saline to dilute fresh vials of stock recombinant TMEM2 protein (200 U per 1.2 mL) at each visit. Subjects return for follow-up 1 week after the third and final hyaluronidase injection session, and then for a final follow-up 3 months later. At the final (4-month) visit, if any previous HA filler injection sites remain detectable, recombinant TMEM2 protein is injected again sufficient to dissolve the remaining filler. To determine the effectiveness of recombinant TMEM2 protein at removing HA filler nodules, before the recombinant TMEM2 protein injections at each visit, as well as at the 4-week and 4-month follow-up visits, a blinded dermatologist rates the detectability of each button on a 5-point scale based on visual detection and palpability. Visual detection is defined as the deviation from smoothness or visible bulge when examining the button from the side, both with and without side lighting and with and without 3× to 5× magnification with a handheld magnifier, and palpability is defined as detection of a papule, nodule, or other skin mass (not related to normal skin or underlying normal skin structure) with increasing pressure from light touch to firm pressure while moving the fingerpad tip of the dominant index finger across the site of the placement of the button. Detectability is rated on a 5-point scale as follows: 0=undetectable, 1=faintly perceptible, 2=mildly perceptible, 3=moderately perceptible, 4=very perceptible.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A recombinant protein, comprising a human TMEM2 extracellular domain or a fragment thereof, wherein the recombinant protein has hyaluronidase catalytic activity.

2. The recombinant protein of claim 1, wherein the human TMEM2 extracellular domain comprises one or more amino acid residues from 1000-1280 residues.

3. The recombinant protein of claim 1, wherein the human TMEM2 extracellular domain comprises a complete sequence of amino acids set forth in SEQ ID NO. 20 (SSKYAPDENC) and/or SEQ ID NO. 90 (NGIGLTFASD).

4. The recombinant protein of claim 1, further comprising at least one N-linked sugar moiety.

5. The recombinant protein of claim 1, further comprising a C-terminal linked sequence of amino acids or N-terminal linked sequence of amino acids.

6. The recombinant protein of claim 5, wherein the N-terminal linked sequence of amino acids comprises a complete sequence of amino acids set forth in SEQ ID NO. 8 (DAAQPARRARRTKLGT).

7. The recombinant protein of claim 1, further comprising at least one linked polymer, wherein the polymer is PEG or dextran.

8. The recombinant protein of claim 1, wherein the recombinant protein comprises a complete sequence of amino acids set forth in SEQ ID NO. 1 or at least 90% amino acid sequence identity with a sequence of amino acids set forth in SEQ ID NO. 1.

9. (canceled)

10. A recombinant protein, comprising at least 1279 amino acid residues of human TMEM2 extracellular domain, wherein the recombinant protein does not comprise the complete sequence of amino acids set forth in SEQ ID NO. 1, or the recombinant protein has at least 95% amino acid sequence identity with a sequence of amino acids set forth in SEQ ID NO. 1, and the recombinant protein comprises a complete sequence of amino acids having at least 90% amino acid sequence identity with a sequence of amino acids set forth in SEQ ID NO. 20 (SSKYAPDENC) and/or SEQ ID NO. 90 (NGIGLTFASD).

11. A recombinant protein, comprising at least 1200, 1220, 1240, 1260, or 1278 amino acid residues of human TMEM2 extracellular domain, wherein the recombinant protein does not comprise SEQ ID NO. 7 (APLVHHHHHHALDENLYFQGALA).

12. The recombinant protein of claim 11, wherein the recombinant protein consists of a contiguous sequence of amino acid residues contained within SEQ ID NO. 1 that includes at least amino acids 1-250, 250-500, 501-750, 751-1000 or 1001-1280 of SEQ ID NO. 1, or the recombinant protein has at least 95% amino acid sequence identity with a sequence of amino acids set forth in SEQ ID NO. 1.

13. The recombinant protein of claim 10, wherein the recombinant protein consists of a contiguous sequence of amino acid residues contained within SEQ ID NO. 1 that includes at least amino acids 1-250, 250-500, 501-750, 751-1000 or 1001-1280 of SEQ ID NO. 1.

14. The recombinant protein of claim 12, wherein the recombinant protein has at least 95%, 96% or 97% amino acid sequence identity with a sequence of amino acids set forth in SEQ ID NO. 1.

15. The recombinant protein of claim 11, wherein the recombinant protein has less than 100% or less than 98% amino acid sequence identity with a sequence of amino acids set forth in SEQ ID NO. 1.

16. The recombinant protein of claim 1, wherein the recombinant protein comprises a sequence of amino acids set forth as amino acids 1-200, 201-400, 401-600, 601-800, 801-1000, or 1001-1280 of SEQ ID NO. 1; or (ii) the recombinant protein contains amino acid substitutions in the sequence of amino acids set forth as amino acids 1-200, 201-400, 401-600, 601-800, 801-1000, or 1001-1280 of SEQ ID NO:1, whereby the amino acid-substituted recombinant protein consists of a sequence of amino acids that has at least 95% amino acid sequence identity with the sequence of amino acids set forth as amino acids 1-400, 401-800, or 801-1280 of SEQ ID NO. 1.

17. The recombinant protein of claim 1, wherein the recombinant protein comprises at least two, three, four, five, six, seven, eight, nine, ten, 11, 12, 13, 14, 15, 16, 17, 18, or 19 sequences of amino acids set forth as amino acids 1-50, 51-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100, 1101-1150, 1151-1200, or 1201-1280 of SEQ ID NO. 1; and wherein the recombinant protein comprises less than 24, or 25 sequences of amino acids, whereby the recombinant protein consists of a sequence of amino acids that has at least 95%, 96%, 97% or 98% amino acid sequence identity with the sequence of amino acids set forth in SEQ ID NO. 1.

18. (canceled)

19. The recombinant protein of claim 17, wherein the recombinant protein has hyaluronidase catalytic activity of at least 50%, at least 60%, at least 70%, at least 80% or at least 90% of TMEM2 extracellular domain.

20. The recombinant protein of claim 11, wherein the recombinant protein comprises a complete sequence of amino acids having 90% amino acid sequence identity with a sequence of amino acids set forth in SEQ ID NO. 90 (NGIGLTFASD) where D of SEQ ID NO. 90 is replaced with E.

21.-58. (canceled)

59. A method for increasing the diffusion of a therapeutic substance in a subject, comprising: administering to a subject the recombinant protein of claim 1; and a therapeutic substance, whereby the diffusion of the therapeutic substance is increased.

60.-67. (canceled)

68. A method for dissolving hyaluronic acid fillers in a subject, the method comprising: administering to the subject the recombinant protein of claim 1, whereby the dissolution of hyaluronic acid fillers is induced.

69.-76. (canceled)
